SEQUENCE LISTING (41 sequences)

SEQ ID NO: 1 - protein, 7 aa, Artificial Sequence
  Synthetic CDR1 sdAb number 1
ATSNISN

SEQ ID NO: 2 - protein, 7 aa, artificial sequence
  Synthetic CDR2 sdAb number 1
RAESRPL

SEQ ID NO: 3 - protein, 9 aa, artificial sequence
  Synthetic CDR3 sdAb number 1
YMPLVRHKA

SEQ ID NO: 4 - protein, 7 aa, artificial sequence
  Synthetic CDR1 sdAb number 2
DSYNESS

SEQ ID NO: 5 - protein, 7 aa, artificial sequence
  Synthetic CDR2 sdAb number 2
ARGNHPL

SEQ ID NO: 6 - protein, 9 aa, artificial sequence
  Synthetic CDR3 sdAb number 2
SMPMPKWKK

SEQ ID NO: 7 - protein, 7 aa, artificial sequence
  Synthetic CDR1 sdAb number 3
RYYEQSI

SEQ ID NO: 8 - protein, 7 aa, artificial sequence
  Synthetic CDR2 sdAb number 3
EYGGWQH

SEQ ID NO: 9 - protein, 9 aa, artificial sequence
  Synthetic CDR3 sdAb number 3
IRHQNQSMM

SEQ ID NO: 10 - protein, 7 aa, artificial sequence
  Synthetic CDR1 sdAb number 4
YTSRESG

SEQ ID NO: 11 - protein, 7 aa, artificial sequence
  Synthetic CDR2 sdAb number 4
RDQDGIS

SEQ ID NO: 12 - protein, 9 aa, artificial sequence
  Synthetic CDR3 sdAb number 4
MIPLTRNAK

SEQ ID NO: 13 - protein, 7 aa, artificial sequence
  Synthetic CDR1 sdAb number 5
YTFSEET

SEQ ID NO: 14 - protein, 7 aa, artificial sequence
  Synthetic CDR2 sdAb number 5
WNHTFFE

SEQ ID NO: 15 - protein, 9 aa, artificial sesquence
  Synthetic CDR3 sdAb number 5
VTPLPPNKA

SEQ ID NO: 16 - protein, 7 aa, artificial sequence
  Synthetic CDR1 sdAb number 6
RGYTGTT

SEQ ID NO: 17 - protein, 7 aa, artificial sequence
  Synthetic CDR2 sdAb number 6
WTSNQTS

SEQ ID NO: 18 - protein, 9 aa, artificial sequence
  Synthetic CDR3 sdAb number 6
MMPLPRWKG

SEQ ID NO: 19 - protein, 28 aa, artificial sequence
  Synthetic FR1
MAEVQLQASGGGFVQPGGSLRLSCAASG

SEQ ID NO: 20 - protein, 19 aa, artificial sequence
  Synthetic FR2
MGWFRQAPGKEREFVSAIS

SEQ ID NO: 21 - protein, 39 aa, artificial sequence
  Synthetic FR3
YYADSVKGRFTISRDNSKNTVYLQMNSLRAEDTATYYCA

SEQ ID NO: 22 - protein, 12 aa, artificial sequence
  Synthetic FR4
YWGQGTQVTVSS

SEQ ID NO: 23 - protein, 121 aa, artificial sequence
  Synthetic sdAb1
MAEVQLQASGGGFVQPGGSLRLSCAASGATSNISNMGWFRQAPGKEREFVSAISRAESRPLYYADSVKGRFTISRDNSKNTVYLQMNSLRAEDTATYYCAYMPLVRHKAYWGQGTQVTVSS

SEQ ID NO: 24 - protein, 121 aa, artificial sequence
  Synthetic sdAb2
MAEVQLQASGGGFVQPGGSLRLSCAASGDSYNESSMGWFRQAPGKEREFVSAISARGNHPLYYADSVKGRFTISRDNSKNTVYLQMNSLRAEDTATYYCASMPMPKWKKYWGQGTQVTVSS

SEQ ID NO: 25 - protein, 121 aa, artificial sequence
  Synthetic sdAb3
MAEVQLQASGGGFVQPGGSLRLSCAASGRYYEQSIMGWFRQAPGKEREFVSAISEYGGWQHYYADSVKGRFTISRDNSKNTVYLQMNSLRAEDTATYYCAIRHQNQSMMYWGQGTQVTVSS

SEQ ID NO: 26 - protein, 121 aa, artificial sequence
  Synthetic sdAb4
MAEVQLQASGGGFVQPGGSLRLSCAASGYTSRESGMGWFRQAPGKEREFVSAISRDQDGISYYADSVKGRFTISRDNSKNTVYLQMNSLRAEDTATYYCAMIPLTRNAKYWGQGTQVTVSS

SEQ ID NO: 27 - protein, 121 aa, artificial sequence
  Synthetic sdAb5
MAEVQLQASGGGFVQPGGSLRLSCAASGYTFSEETMGWFRQAPGKEREFVSAISWNHTFFEYYADSVKGRFTISRDNSKNTVYLQMNSLRAEDTATYYCAVTPLPPNKAYWGQGTQVTVSS

SEQ ID NO: 28 - protein, 121 aa, artificial sequence
  Synthetic sdAb6
MAEVQLQASGGGFVQPGGSLRLSCAASGRGYTGTTMGWFRQAPGKEREFVSAISWTSNQTSYYADSVKGRFTISRDNSKNTVYLQMNSLRAEDTATYYCAMMPLPRWKGYWGQGTQVTVSS

SEQ ID NO: 29 - protein, 1255 aa, Homo sapiens [sequence omitted]

SEQ ID NO: 30 - DNA, 363 bp, artificial sequence
  Synthetic sdA1
atggcggaagtgcagctgcaggcttccgggggaggatttgtgcagccgggggggtcattgcgactgagctgcgccgcatccggagcaacatcaaacatcagtaacatgggctggtttcgtcaggcccctggcaaggagagagagttcgtttccgccatctcccgtgcagaatcgcgtcctctgtattacgctgacagcgtaaagggaagatttacaattagccgggataactccaaaaacacggtctatctccagatgaacagcctcagggccgaggacacagctacgtattactgtgcatatatgcctctggttcgtcacaaggcatactggggacaggggacgcaggtaactgtgagtagc

SEQ ID NO: 31 - protein, 363 aa, artificial sequence
  Synthetic sdAb2
ATGGCGGAAGTGCAGCTGCAGGCTTCCGGGGGAGGATTTGTGCAGCCGGGGGGGTCATTGCGACTGAGCTGCGCCGCATCCGGAGCAACATCAAACATCAGTAACATGGGCTGGTTTCGTCAGGCCCCTGGCAAGGAGAGAGAGTTCGTTTCCGCCATCTCCCGTGCAGAATCGCGTCCTCTGTATTACGCTGACAGCGTAAAGGGAAGATTTACAATTAGCCGGGATAACTCCAAAAACACGGTCTATCTCCAGATGAACAGCCTCAGGGCCGAGGACACAGCTACGTATTACTGTGCATATATGCCTCTGGTTCGTCACAAGGCATACTGGGGACAGGGGACGCAGGTAACTGTGAGTAGC

SEQ ID NO: 32 - DNA, 363 bp, artificial sequence
  Synthetic sdAb3
atggcggaagtgcagctgcaggcttccgggggaggatttgtgcagccggggtggtcattgcgactgagctgcgccgcatccggacgttactacgagcagagtattatgggctggtttcgtcaggcccctggcaaggagagagagttcgtttccgccatctccgaatatggtggttggcagcattactacgctgacagcgtaaagggaagatttacaattagccgggataactccaaaaacacggtctatctccagatgaacagcctcagggccgaggacacagctacgtattactgtgcaattcgtcaccagaaccagtcgatgatgtattggggacaggggacgcaggtaactgtgagtagc

SEQ ID NO: 33 - DNA, 363 bp, artificial sequence
  Synthetic sdAb4
atggcggaagtgcagctgcaggcttccgggggaggatttgtgcagccgggggggtcattgcgactgagctgcgccgcatccggctatacttcacgtgagtccggtatgggctggtttcgtcaggcccctggcaaggagagagagttcgtttccgccatctcccgtgaccaagacggtatttcgtactacgctgacagcgtaaagggaagatttacaattagccgggataactccaaaaacacggtctatctccagatgaacagcctcagggccgaggacacagctacgtattactgtgccatgattcctctgacacgtaacgcaaagtattggggacaggggacgcaggtaactgtgagtagc

SEQ ID NO: 34 - DNA, 363 bp, artificial sqeuence
  Synthetic sdAb5
atggcggaagtgcagctgcaggcttccgggggaggatttgtgcagccgggggggtcattgcgactgagctgcgccgcatccggctatactttcagtgaggagacaatgggctggtttcgtcaggcccctggcaaggagagagagttcgtttccgccatctcgtggaaccatacattttttgagtattacgctgacagcgtaaagggaagatttacaattagccgggataactccaaaaacacggtctatctccagatgaacagcctcagggccgaggacacagctacgtattactgtgccgttacacctctgcctcctaacaaggcatattggggacaggggacgcaggtaactgtgagtagc

SEQ ID NO: 35 - DNA, 363 bp, artificial sequence
  Synthetic sdAb6
atggcggaagtgcagctgcaggcttccgggggaggatttgtgcagccgggggggtcattgcgactgagctgcgccgcatccggacgtggatatacaggtactacaatgggctggtttcgtcaggcccctggcaaggagagagagttcgtttccgccatctcgtggacatcgaaccagacatcgtactacgctgacagcgtaaagggaagatttacaattagccgggataactccaaaaacacggtctatctccagatgaacagcctcagggccgaggacacagctacgtattactgtgcaatgatgcctctgcctcgttggaagggttattggggacaggggacgcaggtaactgtgagtagc

SEQ ID NO: 36 - DNA, 1235 bp, artificial sequence
  Synthetic anti-HER2 hsdAb1 41BB-z (myc tag)
atggccttaccagtgaccgccttgctcctgccgctggccttgctgctccacgccgccaggcattctagagcggaagtgcagctgcaggcttccgggggaggatttgtgcagccgggggggtcattgcgactgagctgcgccgcatccggagcaacatcaaacatcagtaacatgggctggtttcgtcaggcccctggcaaggagagagagttcgtttccgccatctcccgtgcagaatcgcgtcctctgtattacgctgacagcgtaaagggaagatttacaattagccgggataactccaaaaacacggtctatctccagatgaacagcctcagggccgaggacacagctacgtattactgtgcatatatgcctctggttcgcacaaggcatactggggacaggggacgcaggtaactgtgagtagccctgcaggagagcagaagctgatctcagaggaggacctgcatatgaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcaccggtggccacgttgttgaaggactggctggggaacttgaacaacttcgtgcacgactggagcatcacccacaaggtcaacgtgaaccatga

SEQ ID NO: 37 - DNA, 1251 bp, artificial sequence
  Synthetic anti-HER2 hsdAb1 41BB-z (alfa tag)
atggccttaccagtgaccgccttgctcctgccgctggccttgctgctccacgccgccaggcattctagagcggaagtgcagctgcaggcttccgggggaggatttgtgcagccgggggggtcattgcgactgagctgcgccgcatccggagcaacatcaaacatcagtaacatgggctggtttcgtcaggcccctggcaaggagagagagttcgtttccgccatctcccgtgcagaatcgcgtcctctgtattacgctgacagcgtaaagggaagatttacaattagccgggataactccaaaaacacggtctatctccagatgaacagcctcagggccgaggacacagctacgtattactgtgcatatatgcctctggttcgtcacaaggcatactggggacaggggacgcaggtaactgtgagtagccctgcaggacccagcagactggaggaggagctgagaagaagactgaccgagccccatatgaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcaccggtggccacgttgttgaaggactggctggggaacttgaacaacttcgtgcacgactggagcatcacccacaaggtcaacgtgaaccatga

SEQ ID NO: 38 - DNA, 1140 bp, artificial sequence
  Synthetic anti HER2 sdAb1 DAP10z
atggccttaccagtgaccgccttgctcctgccgctggccttgctgctccacgccgccaggcattctagagcggaagtgcagctgcaggcttccgggggaggatttgtgcagccgggggggtcattgcgactgagctgcgccgcatccggagcaacatcaaacatcagtaacatgggctggtttcgtcaggcccctggcaaggagagagagttcgtttccgccatctcccgtgcagaatcgcgtcctctgtattacgctgacagcgtaaagggaagatttacaattagccgggataactccaaaaacacggtctatctccagatgaacagcctcagggccgaggacacagctacgtattactgtgcatatatgcctctggttcgtcacaaggcatactggggacaggggacgcaggtaactgtgagtagccctgcaggagagcagaagctgatctcagaggaggacctgggccggccacagacgactccaggagagagatcatcactccctgccttttaccctggcacttcaggctcttgttccggatgtgggtccctctctctgccgctcctggcaggcctcgtggctgctgatgcggtggcatcgctgctcatcgtgggggcggtgttcctgtgcgcacgcccacgccgcagccccgcccaagaagatggcaaagtctacatcaacatgccaggcaggggccttaagagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcaccggtggccacgttgttgaaggactggctggggaacttgaacaacttcgtgcacgactggagcatcacccacaaggtcaacgtgaaccatga

SEQ ID NO: 39 - DNA, 837 bp, artificial sequence
  Synthetic HER2 sdAb1 DAP12 CAR
atggccttaccagtgaccgccttgctcctgccgctggccttgctgctccacgccgccaggcattctagagcggaagtgcagctgcaggcttccgggggaggatttgtgcagccgggggggtcattgcgactgagctgcgccgcatccggagcaacatcaaacatcagtaacatgggctggtttcgtcaggcccctggcaaggagagagagttcgtttccgccatctcccgtgcagaatcgcgtcctctgtattacgctgacagcgtaaagggaagatttacaattagccgggataactccaaaaacacggtctatctccagatgaacagcctcagggccgaggacacagctacgtattactgtgcatatatgcctctggttcgtcacaaggcatactggggacaggggacgcaggtaactgtgagtagccctgcaggagagcagaagctgatctcagaggaggacctgggccggccactccgtcctgtccaggcccaggcccagagcgattgcagttgctctacggtgagcccgggcgtgctggcagggatcgtgatgggagacctggtgctgacagtgctcattgccctggccgtgtacttcctgggccggctggtccctcgggggcgaggggctgcggaggcagcgacccggaaacagcgtatcactgagaccgagtcgccttatcaggagctccagggtcagaggtcggatgtctacagcgacctcaacacacagaggccgtattacaaacacgttgttgaaggactggctggggaacttgaacaacttcgtgcacgactggagcatcacccacaaggtcaacgtgaaccatga

SEQ ID NO: 40 - DNA, 1236 bp, artificial sequence
  Synthetic HER2 sdAb number 2 41-z CAR
atggccttaccagtgaccgccttgctcctgccgctggccttgctgctccacgccgccaggcattctagagcggaagtgcagctgcaggcttccgggggaggatttgtgcagccgggggggtcattgcgactgagctgcgccgcatccggagattcctacaacgagagttctatgggctggtttcgtcaggcccctggcaaggagagagagttcgtttccgccatctcggcacgtggtaaccatcctctgtattacgctgacagcgtaaagggaagatttacaattagccgggataactccaaaaacacggtctatctccagatgaacagcctcagggccgaggacacagctacgtattactgtgcatcgatgcctatgcctaagtggaagaagtactggggacaggggacgcaggtaactgtgagtagccctgcaggagagcagaagctgatctcagaggaggacctgcatatgaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcaccggtggccacgttgttgaaggactggctggggaacttgaacaacttcgtgcacgactggagcatcacccacaaggtcaacgtgaaccatga

SEQ ID NO: 41 - DNA, 1596 bp, artificial sequence
  Synthetic scFv CD19 41-z CAR
atggccttaccagtgaccgccttgctcctgccgctggccttgctgctccacgccgccaggccggatatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcacctgcaggagagcagaagctgatctcagaggaggacctgcatatgaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcaccggtggccacgttgttgaaggactggctggggaacttgaacaacttcgtgcacgactggagcatcacccacaaggtcaacgtgaaccatga

(19) United States
(12) Patent Application Publication
(10) Pub. No.: US 2024/0317890 A1
(43) Pub. Date: Sep. 26, 2024

(54) HER2 SINGLE DOMAIN ANTIBODIES VARIANTS AND CARS THEREOF

(71) Applicants: INSTITUT CURIE, Paris (FR); CENTRE NATIONAL DE LA RECHERCHE SCIENTIFIQUE - CNRS -, Paris (FR); HONING BIOSCIENCES, Paris (FR)

(72) Inventors: Franck PEREZ, Paris (FR); Sandrine MOUTEL, Paris (FR); Zélia GOUVEIA, Orly (FR)

(21) Appl. No.: 18/269,615

(22) PCT Filed: Jan. 14, 2022
(86) PCT No.: PCT/EP2022/050767

§ 371 Date: Jun. 26, 2023

(30) Foreign Application Priority Data
Jan. 14, 2021 (EP) 21305040.4

(51) Int. Cl. C07K 16/32; A61K 39/00; C07K 14/705; C07K 14/725; G01N 33/574

(52) U.S. Cl. CPC C07K 16/32; C07K 14/7051; C07K 14/70578; G01N 33/57484; A61K 2039/505; C07K 2317/24; C07K 2317/569; C07K 2317/73; C07K 2317/92; C07K 2319/03

(57) ABSTRACT

The present invention relates to humanized HER2 single domain antibodies and variants thereof and their use in therapy and for cancer diagnosis. The invention most particularly proposes chimeric antigen receptors including said humanized HER2 sdAb in their antigen binding domain and their use in cancer cell therapy.

### FIELD OF THE INVENTION

The present disclosure relates to anti-HER2 single domain antibodies (sdAb) and variant thereof and to their use in diagnostic or in cancer therapy. Said anti HER2-sdAb can typically be linked directly or not to a compound of interest and/or included in chimeric antigen receptor and used in cancer cell therapy, notably cellular cancer therapy.

### DETAILED DESCRIPTION

HER2, also known as ERBB2 (human), proto-oncogene Neu, or even CD340 (cluster of differentiation 340), is a member of the human epidermal growth factor receptor (HER/EGFR/ERBB) family. The overexpression of HER2 is correlated with cell proliferation and tumorigenesis and occurs in various in cancers such as approximately 20% to 30% of breast cancers, about 7% to 34% of gastric cancers and in about 30% of salivary duct carcinomas. HER 2 is further expressed in a variety of other human cancers, such as ovarian, adenocarcinoma of the lung, and aggressive forms of uterine cancer (Burstein H J. The distinctive nature of HER2-positive breast cancers, N Engl J Med. 2005; 353:1652-1654; Ruschoff J et al., HER2 testing in gastric cancer: a practical approach. Mod Pathol. 2012; 25:637-650; Meza-Junco J, Au H J, Sawyer M B. Critical appraisal of trastuzumab in treatment of advanced stomach cancer, Cancer Manag Res. 2011; 3:57-64; Chiosea S I, et al., Molecular characterization of apocrine salivary duct carcinoma. Am J Surg Pathol. 2015; 39:744-752). Her2-positive tumors are generally correlated aggressive cancer forms and a poorer prognosis. Several therapeutic methods have been developed to block Her2 activity to suppress tumor growth, notably monoclonal antibodies (mAbs) such as trastuzumab (Santin A D et al., Trastuzumab treatment in patients with advanced or recurrent endometrial carcinoma overexpressing HER2/neu. Int J Gynecol Obstet. 2008; 102:128-131; Vasconcellos F A et al., Generation and characterization of new HER2 monoclonal antibodies. Acta Histochem. 2013; 115:240-244). Although treatment with trastuzumab and other HER2-directed therapies are associated with significant efficacy, only patients with the highest levels of HER2 expression, representing approximately 20% of breast cancer patients, have the potential to respond. Moreover, many patients expressing high levels of HER2 progress or relapse despite receiving the best HER2-directed treatments, and thus require novel treatment approaches. For some patients also these therapeutics show significant clinical benefits, their efficacy remains variable and modest, for example, with no benefit against Her2-positive head and neck cancer (Pollock N I, Grandis J R., HER2 as a therapeutic target in head and neck squamous cell carcinoma. Clin Cancer Res. 2015; 21:526-533; Wu X, Chen S, Lin L, et al. A Single Domain-Based Anti-Her2 Antibody Has Potent Antitumor Activities. Transl Oncol. 2018; 11(2):366-373). Thus, it is necessary to develop new therapeutic avenues to improve the current Her2-targeting therapy.

Adoptive transfer of chimeric antigen receptor T-cell (CAR-T) therapy is notably one of the potential immunotherapies that have shown great promise for the treatment of hematologic malignancies in a series of dramatic successes in clinical trials. Unfortunately, the breakthrough with CAR-T cell therapy in the treatment of hematologic malignancies is still not well replicated in solid tumors (Y. Guo, Y et al., Chimeric antigen receptor-modified T cells for solid tumors: challenges and prospects, J Immunol Res, 2016; J. Li et al., Chimeric antigen receptor T cell (CAR-T) immunotherapy for solid tumors: lessons learned and strategies for moving forward; J Hematol Oncol, 11 (2018), p. 22). Furthermore, scFvs, which are mostly used in the design of chimeric antigen receptors exhibit a number of characteristics that may negatively impact on the therapeutic efficacy of CAR-Ts. Indeed, scFv are notably characterized by poor expression and stability and are prone to unfolding and aggregation.

Thus, there remains a constant need to improve and diversify current therapeutic tools in oncology to cover not only the diversity of patient profiles but also the significant variability of tumours. This is particularly critical for aggressive tumours related to HER2 overexpression.

### SUMMARY OF THE INVENTION

The present application now provides synthetic humanized single domain antibodies specifically binding to HER2 with a high affinity.

These single domain antibodies (sdAbs) have been shown to both (i) accumulate into and (ii) exhibit high cytotoxicity in particular in solid tumors. Due to their small size and thanks to their high penetration capacity in solid tumors, these antibodies further represent an essential diagnostic tool for tumor detection and monitoring.

Furthermore, the present disclosure provides new chimeric antigen receptors that aim to overcome the current pitfall of CAR T cells adoptive therapy. In particular, the results provided by the inventors demonstrate that the CARs, as now developed by the present disclosure, allow to target solid tumors, such as breast cancer and to achieve high cytotoxicity in vivo, while lowering the toxic side effects that were previously observed with the classical CAR design.

Thus, the present disclosure relates to a single domain antibody (sdAb) directed against HER2 wherein said HER2 sdAb has the following formula FR1-CDR1-FR2-CDR2-FR3-CDR3-FR4, and wherein the CDRs are selected from:
a CDR1 of SEQ ID NO:1; a CDR2 of SEQ ID NO:2 and a CDR3 of SEQ ID NO:3,
a CDR1 of SEQ ID NO:4; a CDR2 of SEQ ID NO:5 and a CDR3 of SEQ ID NO:6,
a CDR1 of SEQ ID NO: 7; a CDR2 of SEQ ID NO:8 and a CDR3 of SEQ ID NO:9,
a CDR1 of SEQ ID NO: 10; a CDR2 of SEQ ID NO:11 and a CDR3 of SEQ ID NO:12,
a CDR1 of SEQ ID NO:13; a CDR2 of SEQ ID NO:14 and a CDR3 of SEQ ID NO:15, or
a CDR1 of SEQ ID NO:16; a CDR2 of SEQ ID NO:17 and a CDR3 of SEQ ID NO:18.

More particularly the present invention relates to a humanized synthetic single domain antibody (hssdAb) directed against HER2 having:
a sequence selected from the group consisting of SEQ ID NO: 23, SEQ ID NO:24, SEQ ID NO:25, SEQ ID NO:26, SEQ ID NO:27, and SEQ ID NO:28;
a sequence having at least 90% identity with a sequence selected from the group consisting of SEQ ID NO: 23, SEQ ID NO:24, SEQ ID NO:25, SEQ ID NO:26, SEQ ID NO:27, and SEQ ID NO:28;
a CDR1 of SEQ ID NO:1; a CDR2 of SEQ ID NO:2 and a CDR3 of SEQ ID NO:3 and further having one or more conservative amino acid modifications in one or more of these CDRs;
a CDR1 of SEQ ID NO:4; a CDR2 of SEQ ID NO:5 and a CDR3 of SEQ ID NO:6 and further having one or more conservative amino acid modifications in one or more of these CDRs.
a CDR1 of SEQ ID NO:7; a CDR2 of SEQ ID NO:8 and a CDR3 of SEQ ID NO:9 and further having one or more conservative amino acid modifications in one or more of these CDRs;
a CDR1 of SEQ ID NO:10; a CDR2 of SEQ ID NO:11 and a CDR3 of SEQ ID NO:12 and further having one or more conservative amino acid modifications in one or more of these CDRs.
a CDR1 of SEQ ID NO:13; a CDR2 of SEQ ID NO:14 and a CDR3 of SEQ ID NO:15 and further having one or more conservative amino acid modifications in one or more of these CDRs; or
a CDR1 of SEQ ID NO:16; a CDR2 of SEQ ID NO:17 and a CDR3 of SEQ ID NO:18 and further having one or more conservative amino acid modifications in one or more of these CDRs.

In some embodiments, the humanized anti-HER2 sdAb can be linked directly or indirectly, covalently or non-covalently to a compound of interest selected from a nucleic acid, a polypeptide or a protein, a virus, a toxin and a chemical entity,
optionally said anti HER2 sdAb is linked directly or indirectly, covalently or non-covalently to a diagnostic compound selected from an enzyme, a fluorophore, a NMR or MRI contrast agent, a radioisotope and a nanoparticle;
optionally said anti HER2 sdAb is linked directly or indirectly, covalently or non-covalently to a therapeutic compound selected from cytotoxic agents, chemotherapeutic agents, radioisotopes, targeted anti-cancer agents, immunotherapeutic agents (such as immunosuppressants or immune stimulators), and lytic peptides.

In some embodiments, the HER sdAb as herein described is fused to an immunoglobulin domain, in particular to an Fc domain.

The present invention also relates to multivalent binding compound comprising at least a first sdAb consisting in a HER sdAb as herein defined and comprising at least a second antigen binding compound directed against an antigen selected from a polypeptide, a protein or a small molecule,
optionally the at least second antigen binding compound is a sdAb binding to the same or different antigen;
optionally the first sdAb is located at the N-terminus of the second sdAb or wherein the first sdAb is located at the C-terminus of the second sdAb.

The present invention further encompasses a chimeric antigen receptor (CAR) comprising (a) an antigen binding domain comprising at least a first sdAb consisting in a HER sdAb as herein defined, (b) a transmembrane domain; and (c) an intracellular domain, optionally wherein the antigen binding domain further comprises and a second sdAb specifically binding to a second antigen.

In preferred embodiments, the sdAb comprises CDRs selected from:
a CDR1 of SEQ ID NO:1; a CDR2 of SEQ ID NO:2 and a CDR3 of SEQ ID NO:3,
a CDR1 of SEQ ID NO: 4; a CDR2 of SEQ ID NO:5 and a CDR3 of SEQ ID NO:6,
a CDR1 of SEQ ID NO: 10; a CDR2 of SEQ ID NO:11 and a CDR3 of SEQ ID NO:12,
a CDR1 of SEQ ID NO:13; a CDR2 of SEQ ID NO:14 and a CDR3 of SEQ ID NO:15, or
a CDR1 of SEQ ID NO:16; a CDR2 of SEQ ID NO:17 and a CDR3 of SEQ ID NO:18.
   or has a sequence selected from:
a sequence selected from the group consisting of SEQ ID NO: 23, SEQ ID NO:24, SEQ ID NO:26, SEQ ID NO:27, and SEQ ID NO:28;
a sequence having at least 90% identity with a sequence selected from the group consisting of SEQ ID NO: 23, SEQ ID NO:24, SEQ ID NO:26, SEQ ID NO:27, and SEQ ID NO:28;
a CDR1 of SEQ ID NO:1; a CDR2 of SEQ ID NO:2 and a CDR3 of SEQ ID NO:3 and further having one or more conservative amino acid modifications in one or more of these CDRs;
a CDR1 of SEQ ID NO:4; a CDR2 of SEQ ID NO:5 and a CDR3 of SEQ ID NO:6 and further having one or more conservative amino acid modifications in one or more of these CDRs.
a CDR1 of SEQ ID NO:10; a CDR2 of SEQ ID NO:11 and a CDR3 of SEQ ID NO:12 and further having one or more conservative amino acid modifications in one or more of these CDRs.
a CDR1 of SEQ ID NO:13; a CDR2 of SEQ ID NO:14 and a CDR3 of SEQ ID NO:15 and further having one or more conservative amino acid modifications in one or more of these CDRs; or
a CDR1 of SEQ ID NO:16; a CDR2 of SEQ ID NO:17 and a CDR3 of SEQ ID NO:18 and further having one or more conservative amino acid modifications in one or more of these CDRs.

In such CAR, the transmembrane domain can be selected from the transmembrane domain of the CD8 domain, the CD3zeta domain, the CD28 transmembrane domain, the DAP10 transmembrane domain, or the DAP12 transmembrane domain, and the intracellular domain can comprise one or more domains derived from the CD28, the OX40, the CD3zeta, the DAP10 and/or the DAP12 intracellular domains.

Such CAR can also comprise one or more additional activation/co-stimulatory domains derived from the CD3-zeta chain, CD28, 4-1 BB (CD137), OX40 (CD134), LAG3, TRIM, HVEM, ICOS, CD27, and/or CD40L.

In a multivalent binding compound or a CAR or the present invention, the second antigen can be an HER2 antigen (with a different epitope as for the the first binding compound) or can selected from the group consisting of antigens other than HER2 selected from PSMA, PSCA, BCMA, CS1, GPC3, CSPG4, EGFR, HER3, CA125, CD123, 5T4, IL-13R, CD2, CD3, CD16 (FcγRIII), CD19, CD20, CD22, CD33, CD23, L1 CAM, MUC16, ROR1, SLAMF7, cKit, CD38, CD53, CD71, CD74, CD92, CD100, CD123, CD138, CD146 (MUC18), CD148, CD150, CD200, CD261, CD262, CD362, ROR1, mesothelin, CD33/IL3Ra, c-Met, Glycolipid F77, EGFRvlll, MART-1, gp100, GD-2, O-GD2, NKp46 receptor, presented antigens like NY-ESO-1 or MAGE A3, human telomerase reverse transcriptase (hTERT), survivin, cytochrome P450 1 B1 (CY1 B), Wilm's tumor gene 1 (WT1), livin, alphafetoprotein (AFP), carcinoembryonic antigen (CEA), mucin 16, MUC1, p53, cyclin, and an immune checkpoint target or combinations thereof.

In some embodiments of CARs of the present invention the transmembrane domain is selected from CD8, CD28, DAP10 and DAP12 and the intracellular domain comprises one or more domains derived from the group selected from the CD3 zeta chain intracellular domain, the CD28 intracellular domain, the 4-1BB intracellular domain; the DAP10 intracellular domain or the DAP12 intracellular domain. More particularly, the CAR can comprise:
the full DAP12 protein, or a fragment thereof having at least 90% identity with the DAP12 protein,
the full DAP10 protein or a fragment thereof having at least 90% identity with the DAP10 protein and the CD3zeta intracellular domain, or
the 4-1BB and CD3 zeta intracellular domains.

The present invention also includes an isolated nucleic acid comprising a nucleic acid sequence encoding the humanized anti-HER2 sdAb, the multivalent binding compound or the CAR, vectors comprising thereof and host cells comprising saud nucleic acids and/or vectors.

The present invention includes isolated cell or population of cells expressing the humanized anti-HER2 SdAb, the multivalent binding compound or the CAR as herein described, wherein the cell is typically an immune cell, and more particularly wherein the immune cell is selected from macrophages, NK cells, CD4+/CD8+, TILs/tumor derived CD8 T cells, central memory CD8+ T cells, Treg, MAIT, and Yδ T cells.

The humanized anti-HER2 SdAb, the CAR, the nucleic acid, the vector, the host cell, the isolated cell or cell population can be used in therapy, in particular in the treatment of cancer in a subject in need thereof. More particularly, the humanized anti-HER2 SdAb, the CAR, the nucleic acid, the vector, the host cell, the isolated cell or the cell population can be used in cancer cell therapy. In such embodiment, the cell can be allogenic or autologous.

In some embodiments, humanized anti-HER2 sdAb, multivalent binding compound, CAR, nucleic acid, vector host cell, isolated cell or cell population used in therapy as above mentioned are administered in combination with at least one further therapeutic agent, wherein said at least one further therapeutic agent is an anticancer agent, optionally a chemotherapeutic agent, or an immunotherapeutic agent, optionally a checkpoint inhibitor.

The present invention also encompasses the use of a humanized anti-HER2 SdAb as herein defined for the detection or monitoring of an HER2-mediated cancer.

Thus, the present invention includes an in vitro or ex vivo method for diagnosing or monitoring an HER2 mediated cancer in a subject comprising the steps of:
a) Contacting in vitro an appropriate sample from said subject with a humanized anti-HER2 sdAb of the present disclosure linked to a diagnostic compound, and
b) Determining the expression of HER2 in said sample.

### DETAILED DESCRIPTION

### Definitions

The terminology used herein is for the purpose of describing particular embodiments only and is not intended to be exhaustive. It must be noted that, as used in the specification and the appended claims, the singular forms “a,” “an” and “the” include plural referents unless the context clearly dictates otherwise.

The term “comprising” as used herein is synonymous with “including” or “containing» and is inclusive or open-ended and does not exclude additional, uncited members, elements or method steps.

Unless specifically stated or obvious from context, as used herein, the term “about” is to be understood as within a range of normal tolerance in the art, for example within 2 standard deviations of the mean. About can be understood as within 20%, 15%, 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

As used herein, the term “isolated” refers to a substance or entity that has been (1) separated from at least some of the components with which it was associated when initially produced (whether in nature or in an experimental setting), and (2) produced, prepared, and/or manufactured by the hand of man. Isolated substances and/or entities may be separated from at least about 10%, about 20%, about 30%, about 40%, about 50%, about 60%, about 70%, about 80%, about 90%, or more of the other components with which they were initially associated. In some embodiments, isolated agents are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components.

The “isolated” products of the present disclosure, including isolated nucleic acids, proteins, polypeptides, and antibodies are not products of nature (i.e., “non-naturally occurring”). Rather, the “isolated” nucleic acids, proteins, polypeptides, and antibodies of the present disclosure are “man-made” products. The “isolated” products of the present disclosure can be “markedly different” or “significantly different” from products of nature. By way of a non-limiting example, the isolated nucleic acids may be purified, recombinant, synthetic, labeled, and/or attached to a solid substrate. Such nucleic acids can be markedly different or significantly different than nucleic acids that occur in nature. By way of further non-limiting example, the “isolated” proteins, polypeptides, and antibodies of the present disclosure may be purified, recombinant, synthetic, labeled, and/or attached to a solid substrate. Such proteins, polypeptides, and antibodies can be markedly different or significantly different from proteins, polypeptides, and antibodies that occur in nature.

The term “polynucleotide”, “nucleic acid molecule”, “nucleic acid”, or “nucleic acid sequence” refers to a polymeric form of nucleotides of at least 10 bases in length. The term includes DNA molecules (e.g., cDNA or genomic or synthetic DNA) and RNA molecules (e.g., mRNA or synthetic RNA), as well as analogs of DNA or RNA containing non-natural nucleotide analogs, non-native internucleoside bonds, or both. The nucleic acid can be in any topological conformation. For instance, the nucleic acid can be single-stranded, double-stranded, triple-stranded, quadruplexed, partially double-stranded, branched, hairpinned, circular, or in a padlocked conformation. The nucleic acid (also referred to as polynucleotides) may include both sense and antisense strands of RNA, cDNA, genomic DNA, and synthetic forms and mixed polymers of the above. They may be modified chemically or biochemically or may contain non-natural or derivatized nucleotide bases, as will be readily appreciated by those of skill in the art. Such modifications include, for example, labels, methylation, substitution of one or more of the naturally occurring nucleotides with an analog, internucleotide modifications such as uncharged linkages (e.g., methyl phosphonates, phosphotriesters, phosphoramidates, carbamates, etc.), charged linkages (e.g., phosphorothioates, phosphorodithioates, etc.), pendent moieties (e.g., polypeptides), intercalators (e.g., acridine, psoralen, etc.), chelators, alkylators, and modified linkages (e.g., alpha anomeric nucleic acids, etc.) Also included are synthetic molecules that mimic polynucleotides in their ability to bind to a designated sequence via hydrogen bonding and other chemical interactions. Such molecules are known in the art and include, for example, those in which peptide linkages substitute for phosphate linkages in the backbone of the molecule. Other modifications can include, for example, analogs in which the ribose ring contains a bridging moiety or other structure such as the modifications found in “locked” nucleic acids.

A “synthetic” RNA, DNA or a mixed polymer is one created outside of a cell, for example one synthesized chemically.

The term “nucleic acid fragment” as used herein refers to a nucleic acid sequence that has a deletion, e.g., a 5′-terminal or 3-terminal deletion compared to a full-length reference nucleotide sequence. In an embodiment, the nucleic acid fragment is a contiguous sequence in which the nucleotide sequence of the fragment is identical to the corresponding positions in the naturally-occurring sequence. In some embodiments, fragments are at least 10, 15, 20, or 25 nucleotides long, or at least 20, 30, 40, 50, 60, 70, 80, 90, 100, 1 10, 120, 130, 140, or 150 nucleotides long. In some embodiments a fragment of a nucleic acid sequence is a fragment of an open reading frame sequence. In some embodiments such a fragment encodes a polypeptide fragment (as defined herein) of the protein encoded by the open reading frame nucleotide sequence.

The nucleic acid can be purified. Preferably, the purified nucleic acid is more than 50%, 75%, 85%, 90%, 95%, 97%, 98%, or 99% pure. Within the context of the present disclosure, a purified nucleic acid that is at least 50% pure means a purified nucleic acid sample containing less than 50% other nucleic acids. For example, a sample of a plasmid can be at least 99% pure if it contains less than 1% contaminating bacterial DNA.

The term “operably linked” in the context of nucleic acids refers to a functional relationship between two or more polynucleotide (e.g., DNA) segments. Typically, it refers to the functional relationship of a transcriptional regulatory sequence to a transcribed sequence. For example, a promoter or enhancer sequence is operably linked to a coding sequence if it stimulates or modulates the transcription of the coding sequence in an appropriate host cell or other expression system. Generally, promoter transcriptional regulatory sequences that are operably linked to a transcribed sequence are physically contiguous to the transcribed sequence, i.e., they are cis-acting. However, some transcriptional regulatory sequences, such as enhancers, need not be physically contiguous or located in close proximity to the coding sequences whose transcription they enhance.

The terms “polypeptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer. Unless otherwise indicated, a particular polypeptide sequence also implicitly encompasses conservatively modified variants thereof. Further, a polypeptide may comprise a number of different domains each of which having one or more distinct activities. For the avoidance of doubt, a “polypeptide” may be any length greater two amino acids.

The term “peptide” as used herein refers to a short polypeptide, e.g., one that typically contains less than about 50 amino acids and more typically less than about 30 amino acids. The term as used herein encompasses analogs and mimetics that mimic structural and thus biological function.

The term “isolated protein” or “isolated polypeptide” is a protein or polypeptide that by virtue of its origin or source of derivation (1) is not associated with naturally associated components that accompany it in its native state, (2) exists in a purity not found in nature, where purity can be adjudged with respect to the presence of other cellular material (e.g., is free of other proteins from the same species) (3) is expressed by a cell from a different species, or (4) does not occur in nature (e.g., it is a fragment of a polypeptide found in nature or it includes amino acid analogs or derivatives not found in nature or linkages other than standard peptide bonds). Thus, a polypeptide that is chemically synthesized or synthesized in a cellular system different from the cell from which it naturally originates will be “isolated” from its naturally associated components. A polypeptide or protein may also be rendered substantially free of naturally associated components by isolation, using protein purification techniques well known in the art. As thus defined, “isolated” does not necessarily require that the protein, polypeptide, peptide or oligopeptide so described has been physically removed from a cell in which it was synthesized.

The protein or polypeptide can be purified. Preferably, the purified protein or polypeptide is more than 50%, 75%, 85%, 90%, 95%, 97%, 98%, or 99% pure. Within the context of the present disclosure, a purified protein that is more than 50% (etc.) pure means a purified protein sample containing less than 50% (etc.) other proteins. For example, a sample of a protein comprising can be 99% pure if it contains less than 1% contaminating host cell proteins.

The term “polypeptide fragment” as used herein refers to a polypeptide that has a deletion, e.g., an amino-terminal and/or carboxy-terminal deletion compared to a full-length polypeptide, such as a naturally occurring protein. In an embodiment, the polypeptide fragment is a contiguous sequence in which the amino acid sequence of the fragment is identical to the corresponding positions in the naturally-occurring sequence. Fragments typically are at least 5, 6, 7, 8, 9 or 10 amino acids long, or at least 12, 14, 16 or 18 amino acids long, or at least 20 amino acids long, or at least 25, 30, 35, 40 or 45, amino acids, or at least 50 or 60 amino acids long, or at least 70 amino acids long, or at least 100 amino acids long.

The terms “percent identical” or “percent identity,” in the context of two or more nucleic acids or polypeptide sequences, refers to the extent to which two or more sequences or subsequences that are the same. Two sequences are “identical” if they have the same sequence of amino acids or nucleotides over the region being compared. Two sequences are “substantially identical” if two sequences have a specified percentage of amino acid residues or nucleotides that are the same (i.e., 60% identity, optionally 65%, 70%, 75%, 80%, 85%, 90%, 91% 92%, 93%, 94%, 95%, 96%, 97%, 98% or 99% identity over a specified region, or, when not specified, over the entire sequence), when compared and aligned for maximum correspondence over a comparison window, or designated region as measured using one of the following sequence comparison algorithms or by manual alignment and visual inspection. Optionally, the identity exists over a region that is at least about 30 nucleotides (or 10 amino acids) in length, or more preferably over a region that is 100 to 500 or 1000 or more nucleotides (or 20, 50, 200 or more amino acids) in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well known in the art. Optimal alignment of sequences for comparison can be conducted, e.g., by the local homology algorithm of Smith and Waterman, Adv. Appl. Math. 2:482c (1970), by the homology alignment algorithm of Needleman and Wunsch, J. Mol. Biol. 48:443 (1970), by the search for similarity method of Pearson and Lipman, Proc. Natl. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, WI), or by manual alignment and visual inspection (see, e.g., Brent et al., Current Protocols in Molecular Biology, 2003).

Two examples of algorithms that are suitable for determining percent sequence identity and sequence similarity are the BLAST and BLAST 2.0 algorithms, which are described in Altschul et al., Nuc. Acids Res. 25:3389-3402, 1977; and Altschul et al., J. Mol. Biol. 215:403-410, 1990, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) or 10, M=5, N=−4 and a comparison of both strands. For amino acid sequences, the BLASTP program uses as defaults a word length of 3, and expectation (E) of 10, and the BLOSUM62 scoring matrix (see Henikoff and Henikoff, (1989) Proc. Natl. Acad. Sci. USA 89: 10915) alignments (B) of 50, expectation (E) of 10, M=5, N=−4, and a comparison of both strands. The BLAST algorithm also performs a statistical analysis of the similarity between two sequences (see, e.g., Karlin and Altschul, Proc. Natl. Acad. Sci. USA 90:5873-5787, 1993). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

The percent identity between two amino acid sequences can also be determined using the algorithm of E. Meyers and W. Miller, Comput. Appl. Biosci. 4: 11-17, 1988) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch, J. Mol. Biol. 48:444-453, 1970) algorithm which has been incorporated into the GAP program in the GCG software package (available at www.gcg.com), using either a Blossom 62 matrix or a PAM250 matrix, and a gap weight of 16, 14, 12, 10, 8, 6, or 4 and a length weight of 1, 2, 3, 4, 5, or 6.

Other than percentage of sequence identity noted above, another indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

As used herein a “functional variant” or a given protein includes the wild-type version of said protein, a variant protein belonging to the same family, an homolog protein, or a truncated version, which preserves the functionality of the given protein. Typically the functional variant exhibit at least 70%, 75%, 80%, 85%, 90%, 95%, 97%, 98%, or 99% amino acid identity with the given protein.

As used herein, the term “mammal” refers to any member of the taxonomic class mammalian, including placental mammals and marsupial mammals. Thus, “mammal” includes humans, primates, livestock, and laboratory mammals. Exemplary mammals include a rodent, a mouse, a rat, a rabbit, a dog, a cat, a sheep, a horse, a goat, a llama, cattle, a primate, a pig, and any other mammal. In some embodiments, the mammal is at least one of a transgenic mammal, a genetically-engineered mammal, and a cloned mammal.

According to the present disclosure, the term “disease” refers to any pathological state, including cancer diseases, in particular those forms of cancer diseases described herein.

The term “normal” refers to the healthy state or the conditions in a healthy subject or tissue, i.e., non-pathological conditions, wherein “healthy” preferably means non-cancerous.

The term “malignancy” refers to a non-benign tumor or a cancer. As used herein, the term “cancer” includes a malignancy characterized by deregulated or uncontrolled cell growth.

The term “cancer” includes primary malignant tumors (e.g., those whose cells have not migrated to sites in the subject's body other than the site of the original tumor) and secondary malignant tumors (e.g., those arising from metastasis, the migration of tumor cells to secondary sites that are different from the site of the original tumor).

Cancers are classified by the type of cell that resembles the tumor and, therefore, the tissue presumed to be the origin of the tumor. These are the histology and the location, respectively.

The term “cancer” according to the disclosure comprises notably leukemias, seminomas, melanomas, teratomas, lymphomas, neuroblastomas, gliomas and sarcomas. The term cancer notably include rectal cancer, endometrial cancer, kidney cancer, adrenal cancer, thyroid cancer, blood cancer, skin cancer, cancer of the brain, cervical cancer, intestinal cancer, liver cancer, colon cancer, stomach cancer, intestine cancer, head and neck cancer, gastrointestinal cancer, lymph node cancer, esophagus cancer, colorectal cancer, pancreas cancer, ear, nose and throat (ENT) cancer, breast cancer, prostate cancer, cancer of the uterus, ovarian cancer and lung cancer, soft tissue tumors and the metastases thereof. The term cancer according to the present disclosure also comprises cancer metastases and relapse of cancer.

“Growth of a tumor” or “tumor growth” according to the present disclosure relates to the tendency of a tumor to increase its size and/or to the tendency of tumor cells to proliferate.

For purposes of the present disclosure, the terms “cancer” and “cancer disease” are used interchangeably with the terms “tumor” and “tumor disease”.

By “treat” is meant to administer a compound or composition as described herein to a subject in order to prevent or eliminate a disease, including reducing the size of a tumor or the number of tumors in a subject; arrest or slow a disease in a subject; inhibit or slow the development of a new disease in a subject; decrease the frequency or severity of symptoms and/or recurrences in a subject who currently has or who previously has had a disease; and/or prolong, i.e. increase the lifespan of the subject. In particular, the term “treatment of a disease” includes curing, shortening the duration, ameliorating, preventing, slowing down or inhibiting progression or worsening, or preventing or delaying the onset of a disease or the symptoms thereof.

The therapeutically active agents or product, vaccines and compositions described herein may be administered via any conventional route, including by injection or infusion.

The agents described herein are administered in effective amounts. An “effective amount” refers to the amount which achieves a desired reaction or a desired effect alone or together with further doses. In the case of treatment of a particular disease or of a particular condition, the desired reaction preferably relates to inhibition of the course of the disease. This comprises slowing down the progress of the disease and, in particular, interrupting or reversing the progress of the disease. The desired reaction in a treatment of a disease or of a condition may also be delay of the onset or a prevention of the onset of said disease or said condition. An effective amount of an agent described herein will depend on the condition to be treated, the severity of the disease, the individual parameters of the patient, including age, physiological condition, size and weight, the duration of treatment, the type of an accompanying therapy (if present), the specific route of administration and similar factors. Accordingly, the doses administered of the agents described herein may depend on several of such parameters. In the case that a reaction in a patient is insufficient with an initial dose, higher doses (or effectively higher doses achieved by a different, more localized route of administration) may be used.

The pharmaceutical compositions as herein described are preferably sterile and contain an effective amount of the therapeutically active substance to generate the desired reaction or the desired effect.

The pharmaceutical compositions as herein described are generally administered in pharmaceutically compatible amounts and in pharmaceutically compatible preparation. The term “pharmaceutically compatible” refers to a nontoxic material which does not interact with the action of the active component of the pharmaceutical composition. Preparations of this kind may usually contain salts, buffer substances, preservatives, carriers, supplementing immunity-enhancing substances such as adjuvants, e.g. CpG oligonucleotides, cytokines, chemokines, saponin, GM-CSF and/or RNA and, where appropriate, other therapeutically active compounds. When used in medicine, the salts should be pharmaceutically compatible.

### Single Domain Antibodies and Variants Thereof

As used herein, the term “HER2” has its general meaning in the art and includes human HER2 (also named “Receptor tyrosine-protein kinase erbB-2”), in particular the native-sequence polypeptide, isoforms, chimeric polypeptides, all homologs, fragments, and precursors of human HER2. The amino acid sequence for native HER2 includes the UniProt reference P04626 (ERBB2_HUMAN).

More specifically the term “HER2” includes the human HER2 of the following SEQ ID:29

>sp|P04626|ERBB2_HUMAN Receptor tyrosine-
protein kinase erbB-2 OS = Homo sapiens
OX = 9606 GN = ERBB2 PE = 1 SV = 1
MELAALCRWGLLLALLPPGAASTQVCTGTDMKLRLPASPETHLDML
RHLYQGCQVVQGNLELTYLPTNASLSFLQDIQEVQGYVLIAHNQV
RQVPLQRLRIVRGTQLFEDNYALAVLDNGDPLNNTTPVTGASPGG
LRELQLRSLTEILKGGVLIQRNPQLCYQDTILWKDIFHKNNQLAL
TLIDTNRSRACHPCSPMCKGSRCWGESSEDCQSLTRTVCAGGCAR
CKGPLPTDCCHEQCAAGCTGPKHSDCLACLHFNHSGICELHCPAL
VTYNTDTFESMPNPEGRYTFGASCVTACPYNYLSTDVGSCTLVCP
LHNQEVTAEDGTQRCEKCSKPCARVCYGLGMEHLREVRAVTSANI
QEFAGCKKIFGSLAFLPESFDGDPASNTAPLQPEQLQVFETLEEI
TGYLYISAWPDSLPDLSVFQNLQVIRGRILHNGAYSLTLQGLGIS
WLGLRSLRELGSGLALIHHNTHLCFVHTVPWDQLFRNPHQALLHT
ANRPEDECVGEGLACHQLCARGHCWGPGPTQCVNCSQFLRGQECV
EECRVLQGLPREYVNARHCLPCHPECQPQNGSVTCFGPEADQCVA
CAHYKDPPFCVARCPSGVKPDLSYMPIWKFPDEEGACQPCPINCT
HSCVDLDDKGCPAEQRASPLTSIISAVVGILLVVVLGVVFGILIK
RRQQKIRKYTMRRLLQETELVEPLTPSGAMPNQAQMRILKETELR
KVKVLGSGAFGTVYKGIWIPDGENVKIPVAIKVLRENTSPKANKE
ILDEAYVMAGVGSPYVSRLLGICLTSTVQLVTQLMPYGCLLDHVR
ENRGRLGSQDLLNWCMQIAKGMSYLEDVRLVHRDLAARNVLVKSP
NHVKITDFGLARLLDIDETEYHADGGKVPIKWMALESILRRRFTH
QSDVWSYGVTVWELMTFGAKPYDGIPAREIPDLLEKGERLPQPPI
CTIDVYMIMVKCWMIDSECRPRFRELVSEFSRMARDPQRFVVIQN
EDLGPASPLDSTFYRSLLEDDDMGDLVDAEEYLVPQQGFFCPDPA
PGAGGMVHHRHRSSSTRSGGGDLTLGLEPSEEEAPRSPLAPSEGA
GSDVFDGDLGMGAAKGLQSLPTHDPSPLQRYSEDPTVPLPSETDG
YVAPLTCSPQPEYVNQPDVRPQPPSPREGPLPAARPAGATLERPK
TLSPGKNGVVKDVFAFGGAVENPEYLTPQGGAAPQPHPPPAFSPA
FDNLYYWDQDPPERGAPPSTFKGTPTAENPEYLGLDVPV

The term “antibody”, broadly refers to any immunoglobulin (Ig) molecule, or antigen binding portion thereof, comprised of four polypeptide chains, two heavy (H) chains and two light (L) chains, or any functional fragment, mutant, variant, or derivation thereof, which retains the essential epitope binding features of an Ig molecule. Such mutant, variant, or derivative antibody formats are known in the art. In a full-length antibody, each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. Immunoglobulin molecules can be of any type (e.g., IgG, IgE, IgM, IgD, IgA and IgY), class (e.g., IgG 1, lgG2, IgG 3, lgG4, IgAI and lgA2) or subclass.

An antibody fragment is a portion of an antibody, for example as F(ab′)₂, Fab, Fv, sFv and the like. Functional fragments of a full-length antibody retain the target specificity of a full-length antibody. Recombinant functional antibody fragments, such as Fab (Fragment, antibody), scFv (single chain variable chain fragments) and single domain antibodies (dAbs) have therefore been used to develop therapeutics as an alternative to therapeutics based on mAbs. scFv fragments (˜25 kDa) consist of the two variable domains, VH and VL. Naturally, VH and VL domains are non-covalently associated via hydrophobic interaction and tend to dissociate. However, stable fragments can be engineered by linking the domains with a hydrophilic flexible linker to create a single chain Fv (scFv). The smallest antigen binding fragment is the single variable fragment, namely the VH or VL domain. Binding to a light chain/heavy chain partner respectively is not required for target binding. Such fragments are used in single domain antibodies. A single domain antibody (˜12 to 15 kDa) therefore has either the VH or VL domain.

As used herein the term “single-domain antibody” (sdAb) or Nanobody® (tradename of Ablynx). has its general meaning in the art and refers to an antibody fragment with a molecular weight of only 12-15 kDa consisting of the single heavy chain variable domain of antibodies of the type that can be found in Camelid mammals, and which are naturally devoid of light chains. Thus, in some embodiments, such single-domain antibodies can be VHHs. For a general description of (single) domain antibodies, reference is also made to the prior art cited above, as well as to EP 0 368 684, Ward et al. (Nature 1989 Oct. 12; 341 (6242): 544-6), Holt et al, Trends Biotechnol, 2003, 21(1 1):484-490; and WO 06/030220, WO 06/003388. The amino acid sequence and structure of a single-domain antibody can be considered to be comprised of four framework regions or “FRs” which are referred to in the art and herein as “Framework region 1” or “FR1”; as “Framework region 2” or “FR2”; as “Framework region 3” or “FR3”; and as “Framework region 4” or “FR4” respectively; which framework regions are interrupted by three complementary determining regions or “CDRs”, which are referred to in the art as “Complementary Determining Region 1” or “CDR1”; as “Complementarity Determining Region 2” or “CDR2” and as “Complementarity Determining Region 3” or “CDR3”, respectively. Accordingly, the single-domain antibody can be defined as an amino acid sequence with the general structure: FR1-CDR1-FR2-CDR2-FR3-CDR3-FR4 in which FR1 to FR4 refer to framework regions 1 to 4 respectively, and in which CDR1 to CDR3 refer to the complementarity determining regions 1 to 3. In the context of the present disclosure, the amino acid residues of the single-domain antibody are numbered according to the general numbering for VH domains given by the International ImMunoGeneTics information system amino acid numbering (http://imgt.cmes.fr/).

An “isolated sdAb”, as used herein, refers to a single domain antibody (sdAb) that is substantially free of other antibodies, notably other sdAb having different antigenic specificities (e.g., an isolated antibody that specifically binds to HER2 is substantially free of antibodies that specifically bind to other antigens than HER2). Moreover, an isolated antibody may be substantially free of other cellular material and/or chemicals.

As used herein, the term “synthetic” means that such antibody has not been obtained from fragments of naturally occurring antibodies but produced from recombinant nucleic acids comprising artificial coding sequences.

As used herein the terms anti-HER2 antibody or anti-HER2 sdAb have the same meaning as the terms an antibody, or a sdAb, directed against the HER2 protein, and notably directed against the human HER2 protein of SEQ ID NO:29.

sdAb affinity refers to the strength with which the sdAb binds to the epitope presented on an antigen, such as a HER2 in the present disclosure, through its antigen-binding site (paratope). Affinity may be assessed based on assessment of the K_{D} value.

The term “K_{D}”, as used herein, is intended to refer to the equilibrium dissociation constant, which is obtained from the ratio of k_{off} to kₒₙ (i.e. k_{off}/kₒₙ) and is expressed as a molar concentration (M). The K_{D} value relates to the concentration of antibody (the amount of antibody needed for a particular experiment) and so the lower the K_{D} value (lower concentration) and thus the higher the affinity of the antibody. K_{D} values for antibodies can be determined using methods well established in the art. Methods for determining the K_{D} values of mAbs can be found in Harlow et al., Antibodies: A Laboratory Manual, Cold Spring Harbor Laboratory Press, Cold Spring Harbor, N.Y., 1988; Coligan et al., eds., Current Protocols in Immunology, Greene Publishing Assoc. and Wiley Interscience, N.Y., 1992, 1993, and Muller, Meth. Enzymol. 92:589-601, 1983, which references are entirely incorporated herein by reference. Affinity measurements are generally performed at 25° C. The terms “k_{assoc}” or “ka”, or “kₒₙ” as used herein, is intended to refer to the association rate of a particular antibody-antigen interaction, whereas the terms “k_{dis}” or “kd,”, or k_{off} as used herein, is intended to refer to the dissociation rate of a particular antibody-antigen interaction. A method for determining the K_{D} of an antibody is by using surface plasmon resonance, or by using a biosensor system such as a Biacore® (see also for detailed information regarding affinity assessment Rich R L et al., Anal Biochem, 2001, but also for more details about the specific implementation of affinity measurement for sdAb Moutel S et al., eLife 2016; 5:e16228). Briefly, as sdAb are smaller proteins that their respective antigens, they can be capture on a sensorship from a Biocore biosensor instrument, while the recombinant antigens (i.e., typically rHER2) can be used as analytes. Analytes can be injected sequentially with increased concentration ranging for example between 3.125 nM to 50 nM in a single cycle without regeneration of the sensorship between injections. Binding parameters can be obtained by fitting the overlaid sensorgrams with the 1:1. Langmuir binding model of the BIAevalutation software.

Affinity measurement can also be performed with the bio-layer interferometry (BLI) based Octet biosensor (see also the results for more details). The principle of BLI technology is based on the optical interference pattern of white light reflected from two surfaces—a layer of immobilized protein and an internal reference layer. The binding between a ligand immobilized on the biosensor tip surface and an analyte in solution produces an increase in optical thickness at the biosensor tip, which results in a shift in the interference pattern measured in nanometers. The wavelength shift (Δλ) is a direct measure of the change in optical thickness of the biological layer, when this shift is measured over a period of time and its magnitude plotted as a function of time, a classic association/dissociation curve is obtained. This interaction is measured in real-time, providing the ability to monitor binding specificity, association rate and dissociation rate, and concentration with outstanding precision and accuracy.

In some embodiments, the apparent affinity may be assessed in binding assays using an ELISA assay (with typically hHER2-Fc coated wells) or flow cytometry (typically using cells expressing recombinant HER2, notably hHER2) Typically, a single domain antibody as per the present disclosure binds to HER2, notably human HER2 as herein defined with a K_{D} with a K_{D} binding affinity of about 10⁻⁶ M or less, 10⁻⁷ M or less, 10⁻⁸ M or less, 10⁻⁹ M or less, 10⁻¹⁰ M or less, or 10⁻¹¹ M or less. Preferably the K_{D} binding affinity is comprised between 10⁷ and 10⁻¹⁰ M, between 10⁻⁸ and 1·10⁻¹¹ M, notably 10⁻⁸ and 10⁻¹⁰, notably comprised between 1·10⁻⁹ and 100·10⁻⁹, notably between 1·10⁻⁹ and 10·10⁻⁹, between 1·10⁻⁹ and 5·10⁻⁹, or between 5·10⁻⁹ and 100·10⁻⁹ notably between 10·10⁻⁹ and 100·10⁻⁹, more particularly between 50·10⁻¹⁰ and 100·10⁻⁹.

The inventors have isolated 6 reference single-domain antibodies (sdAb) with the required properties, notably the required affinity and characterized by following sequences:

TABLE 1
Full sdAb sequences.

| sdAb | | | | | | | |
|---|---|---|---|---|---|---|---|
| (Nº) | FR1 | CDR1 | FR2 | CDR2 | FR3 | CDR3 | FR4 |
| 1 | MAEVQLQAS | AT | MGWFRQAP | RA | YYADSVKGRF | YMP | YWGQG |
| | GGGFVQPGG | SNI | GKEREFVS | ESR | TISRDNSKNT | LVRH | TQVTV |
| | SLRLSCAAS | SN | AIS | PL | VYLQMNSLRA | KA | SS |
| | G | | | | EDTATYYCA | | |
| 2 | MAEVQLQAS | DSY | MGWFRQAP | ARG | YYADSVKGR | SMPM | YWGQG |
| | GGGFVQPGG | NES | GKEREFVS | NHP | FTISRDNSK | PKWK | TQVTV |
| | SLRLSCAAS | S | AIS | L | NTVYLQMNS | K | SS |
| | G | | | | LRAEDTATY | | |
| | | | | | YCA | | |
| 3 | MAEVQLQAS | RY | MGWFRQAP | EYG | YYADSVKGR | IRHQ | YWGQG |
| | GGGFVQPGG | YE | GKEREFVS | GW | FTISRDNSK | NQSM | TQVTV |
| | SLRLSCAAS | QSI | AIS | QH | NTVYLQMNS | M | SS |
| | G | | | | LRAEDTATY | | |
| | | | | | YCA | | |
| 4 | MAEVQLQAS | YTS | MGWFRQAP | RD | YYADSVKGR | MIPL | YWGQG |
| | GGGFVQPGG | RES | GKEREFVS | QDG | FTISRDNSK | TRNA | TQVTV |
| | SLRLSCAAS | G | AIS | IS | NTVYLQMNS | K | SS |
| | G | | | | LRAEDTATY | | |
| | | | | | YCA | | |
| 5 | MAEVQLQAS | YTF | MGWFRQAP | WN | YYADSVKGR | VTPLP | YWGQG |
| | GGGFVQPGG | SEE | GKEREFVS | HTFF | FTISRDNSK | PNKA | TQVTV |
| | SLRLSCAAS | T | AIS | E | NTVYLQMNS | | SS |
| | G | | | | LRAEDTATY | | |
| | | | | | YCA | | |
| 6 | MAEVQLQAS | RG | MGWFRQAP | WT | YYADSVKGR | MMPL | YWGQG |
| | GGGFVQPGG | YT | GKEREFVS | SNQ | FTISRDNSK | PRWK | TQVTV |
| | SLRLSCAAS | GT | AIS | TS | NTVYLQMNS | G | SS |
| | G | T | | | LRAEDTATY | | |
| | | | | | YCA | | |

Therefore, the present disclosure encompasses single domain antibodies having at least the 3 CDRs of one of the 6 reference single domain antibodies as defined in table 1.

sdAb no 1 to 6 as detailed in table 1 comprise framework regions including humanized amino acid residues and are therefore referred as humanized sdabs (hsdAbs) or are also named humanized synthetic sdAbs (hssdAbs).

In some embodiments, sdAbs according to the present disclosure include sdAbs having at least 60, 70, 80, 90, 95, 96, 97, 98, 99 or 100 percent identity with the amino acid sequences as set forth in any one of SEQ ID NO:23-28.

sdAbs as per the present disclosure notably include anti-HER2 sdAbs having framework region sequences that have at least 60, 70, 80, 90, 95, 96, 97, 98, 99 or 100 percent identity with one or more of the humanized sequences SEQ ID NO:19-22.

In some embodiments of the present invention, the 3CDR regions of an anti-HER2 sdAb as herein disclosed can be 100% identical to the 3 CDR regions of one of the reference humanized sdAbs (hsdAbs) no 1 to 6 as defined in table 1. Alternatively, in some embodiments, hsdAbs according to the present disclosure may have an amino acid sequence that have been mutated by amino acid deletion, insertion or substitution, notably conservative substitution, yet that have at least 60, 70, 80, 90, 95, 96, 97, 98, 99 or 100 percent identity in the CDR regions compared with the CDR regions of the sdAb of table 1. Typically, as per the present disclosure, antibodies may have between 1, 2, 3 or 4 amino acid variations (including deletion, insertion or substitution—in particular conservative substitution) in one or more of its 3 CDRs, as compared to the respective 3 CDR sequences of the sdAb of the table 1.

In some embodiments, the single domain antibody of the present disclosure is a mutant variant of one of the reference single domain antibodies of table 1, having the 3 CDR regions 100% identical to the corresponding 3 CDR regions of said reference sdAb, and wherein no more than 1, 2, 3, 4 or 5 amino acids have been mutated by amino acid deletion(s), insertion(s) or substitution(s), notably conservative substitution(s), in one or more of the FR1, FR2, FR3 and/or FR4 regions, when compared with the corresponding framework regions of the corresponding reference sdAb (SEQ ID NO:19-22).

In some embodiments, an sdAb of the present disclosure comprises or consist in a sequence selected from SEQ ID NOs. 23-28 having one or more amino acid substitutions, deletions, insertions or other modifications compared to SEQ ID NOs. 23-28, and which retains a biological function of the single domain antibody. Modifications may include one or more substitution, deletion or insertion of one or more codons encoding the single domain antibody or polypeptide that results in a change in the amino acid sequence as compared with the sequence of the reference single domain antibody or polypeptide. Amino acid substitutions can be the result of replacing one amino acid with another amino acid having similar structural and/or chemical properties, such as the replacement of a leucine with a serine, i.e., conservative amino acid replacements. Insertions or deletions may optionally be in the range of about 1 to 5 amino acids. The variation allowed may be determined by systematically making insertions, deletions or substitutions of amino acids in the sequence and testing the resulting variants for activity exhibited by the full-length or mature native sequence.

In some embodiments, the modification is a conservative sequence modification. As used herein, the term “conservative sequence modifications” is intended to refer to amino acid modifications that do not significantly affect or alter the binding characteristics of the antibody containing the amino acid sequence. Such conservative modifications include amino acid substitutions, additions, and deletions. Modifications can be introduced into single domain antibody as herein described by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine) as well as aliphatic residues (I, L, V, and M), cycloalkenyl-associated residues (F, H, W, and Y), hydrophobic residues (A, C, F, G, H, I, L, M, R, T, V, W, and Y), negatively charged residues (D and E), polar residues (C, D, E, H, K, N, Q, R, S, and T), positively charged residues (H, K, and R), small residues (A, C, D, G, N, P, S, T, and V), very small residues (A, G, and S), residues involved in turn (A, C, D, E, G, H, K, N, Q, R, S, P), and formation T, flexible residues (Q, T, K, S, G, P, D, E, and R).

More conservative substitution groupings include valine-leucine-isoleucine, phenylalanine-tyrosine, lysine-arginine, alanine-valine, and asparagine-glutamine. Conservation in terms of hydropathic/hydrophilic properties and residue weight/size also is substantially retained in a variant as compared to a CDR of the any one of mAbs 1-11. The importance of the hydropathic amino acid index in conferring interactive biologic function on a protein is generally understood in the art. It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. Each amino acid has been assigned a hydropathic index on the basis of their hydrophobicity and charge characteristics these are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophane (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5). The retention of similar residues may also or alternatively be measured by a similarity score, as determined by use of a BLAST program (e.g., BLAST 2.2.8 available through the NCBI using standard settings BLOSUM62, Open Gap=11 and Extended Gap=1).

Thus, one or more amino acid residues within the CDR regions of a single domain antibody of the present disclosure can be replaced with other amino acid residues from the same side chain family and the altered antibody can be tested for retained function (i.e., the functions set forth in (c) through (I) above) using the functional assays described herein.

In some embodiments, the single domain antibody is selected from one of the SEQ ID NOs. 24-29, but comprises one or more amino acid substitutions, for example 1 to 20, such as 1, 2, 3, 4, 5, 6, 7, 8, 9 or 10 amino acid substitutions. The one or more amino acid substitution can be in one or more of the framework areas. Alternatively, or in addition, the one or more amino acid substitution can be in one or more of the CDRs. In some embodiments, the amino acid substitutions are in the framework and CDR sequences.

In some embodiments, the humanized single domain antibody is a variant of a single domain antibody selected from those having SEQ ID NOs. 23-28, that comprises one or more sequence modification while its functional properties are similar (variation less of 15%, notably less than 10%, or less than 5%) to the parent unmodified single domain antibody. More particularly, a variant antibody as herein intended has typically preserved binding affinity for HER2, as well as preferably preserved in vitro cytotoxic activity notably in the CAR format (as illustrated in the results herein).

In some embodiments, said variant has improvements in one or more of a property such as binding affinity, specificity, thermostability, expression level, effector function, glycosylation, reduced immunogenicity, or solubility as compared to the unmodified single domain antibody.

A skilled person will know that there are different ways to identify, obtain and optimise the antigen binding molecules as described herein, including in vitro and in vivo expression libraries. Optimisation techniques known in the art, such as display (e.g., ribosome and/or phage display) and/or mutagenesis (e.g., error-prone mutagenesis) can be used. The present disclosure therefore also comprises sequence optimised variants of the single domain antibodies described herein.

### Single Domain Antibody Engineering

In some embodiments, sdAbs according to the present disclosure can be chemically modified, for example to increase their molecular weight to reduce renal clearance or protect for example from proteases. PEGylation (covalent attachment of a polyethylene glycol (PEG) group) for example has been widely used to increase the half-life. Other strategies to limit renal clearance involve attachment of negative charges to the sdAb, such as addition of sialic acid polymers (polysialylation) or hydroxyethal starch (HESylation) and by fusion with the highly syaliated beta carboxyterminal peptide (CTP) amino acid-residue of the human chorionic gonadotrophin (hCG) hormone.

In some embodiments of the present disclosure, an isolated humanized single domain antibody as herein described can be linked directly or not, covalently or not to a compound of interest. The substance or compound of interest as defined above can be directly and covalently or non-covalently linked to a single domain antibody as herein defined either to one of the terminal ends (N or C terminus), or to the side chain of one of the amino acids of said single domain antibody. The substance of interest can also be indirectly and covalently or non-covalently linked to said single domain antibody by means of a spacer either to one of the terminal ends of said single domain antibody, or to a side chain of one of the amino acids of said single domain antibody.

Conventional linking methods of a substance of interest to a peptide, in particular an antibody, are known in the art (e.g., See Ternynck and Avrameas 1987 “Techniques immunoenzymatiques” Ed. INSERM, Paris; Hermanson, 2010, Bioconjugate Techniques, Academic Press).

In some embodiments, single domain antibodies as herein described can be notably in the form of “antibody drug conjugate” of the formula sdAb-(L-(D)m)n or a pharmaceutically acceptable salt thereof; wherein sdAb is a single domain antibody as previously disclosed; L is a linker; D is a compound of interest; m is an integer from 1 to 8; and n is an integer from 1 to 10, typically equal to 3 or 4.

The term “antibody drug conjugate” as used herein refers to the linkage of a single domain antibody with another agent, such as a chemotherapeutic agent, a toxin, an immunotherapeutic agent, an imaging probe, and the like. The linkage can be covalent bonds, or non-covalent interactions such as through electrostatic forces. Various linkers, known in the art, can be employed in order to form the immunoconjugate. The linker (L) can be for example selected from the group consisting of a cleavable linker, a non-cleavable linker, a hydrophilic linker, a procharged linker and a dicarboxylic acid-based linker.

In some embodiments, the single domain antibody of the present disclosure is conjugated, or covalently linked to the compound of interest. As used herein, the term “conjugation” has its general meaning in the art and means a chemical conjugation, or chemical crosslinking. Many chemical cross-linking methods are also known in the art. Cross-linking reagents may be homobifunctional (i.e., having two functional groups that undergo the same reaction) or heterobifunctional (i.e., having two different functional groups). Numerous cross-linking reagents are commercially available. Detailed instructions for their use are readily available from the commercial suppliers. A general reference on polypeptide cross-linking and conjugate preparation is: WONG, Chemistry of protein conjugation and cross-linking, CRC Press (1991), see also Amon et al., “Monoclonal Antibodies For Immunotargeting Of Drugs In Cancer Therapy,” in Monoclonal Antibodies For Immunotargeting Of Drugs In Cancer Therapy,” in Monoclonal Antibodies And Cancer Therapy (Reisfeld et al. eds., Alan R. Liss, Inc., 1985); Hellstrom et al., “Antibodies For Drug Delivery,” in Controlled Drug Delivery (Robinson et al. eds., Marcel Deiker, Inc., 2nd ed. 1987); Thorpe, “Antibody Carriers Of Cytotoxic Agents In Cancer Therapy: A Review,” in Monoclonal Antibodies '84: Biological And Clinical Applications (Pinchera et al. eds., 1985); “Analysis, Results, and Future Prospective of the Therapeutic Use of Radiolabeled Antibody In Cancer Therapy,” in Monoclonal Antibodies For Cancer Detection And Therapy (Baldwin et al. eds., Academic Press, 1985); and Thorpe et al., 1982, Immunol. Rev. 62: 119-58. See also, e.g., PCT publication WO 89/12624.). Typically, the nucleic acid molecule is covalently attached to lysines or cysteines on the antibody, through N-hydroxysuccinimide ester or maleimide functionality respectively. Methods of conjugation using engineered cysteines or incorporation of unnatural amino acids have been reported to improve the homogeneity of the conjugate (Axup, J. Y., Bajjuri, K. M., Ritland, M., Hutchins, B. M., Kim, C. H., Kazane, S. A., Haider, R., Forsyth, J. S., Santidrian, A. F., Stafin, K., et al. (2012). Synthesis of site-specific antibody-drug conjugates using unnatural amino acids. Proc. Natl. Acad. Sci. USA 109, 16101-16106.; Junutula, J. R., Flagella, K. M., Graham, R. A., Parsons, K. L., Ha, E., Raab, H., Bhakta, S., Nguyen, T., Dugger, D. L., Li, G., et al. (2010) Engineered thio-trastuzumab-DMl conjugate with an improved therapeutic index to target human epidermal growth factor receptor 2-positive breast cancer. Clin. Cancer Res. 16, 4769-4778.). Junutula et al. (2008) developed cysteine-based site-specific conjugation called “THIOMABs” (TDCs) that are claimed to display an improved therapeutic index as compared to conventional conjugation methods. In some embodiments, the single domain antibody of the present disclosure is conjugated to the heterologous moiety by a linker molecule. As used herein, the term “linker molecule” refers to any molecule attached to the single domain antibody the present disclosure. The attachment is typically covalent. In some embodiments, the linker molecule is flexible and does not interfere with the binding of the single domain antibody the present disclosure.

A compound or substance of interest as herein intended can be non-limitatively selected from a nucleic acid, a polypeptide or a protein, a virus, a toxin, a bacteria, and a chemical entity.

In some embodiments, compounds of interest include antigen binding domain agents such as antibodies, variants and fragments thereof, notably the same or another single domain antibody, aptamers, or enzymes.

The compound or substance of interest, as above described, can be a therapeutic or a diagnostic compound. Therapeutic compounds notably include therapeutic compounds having anti-cancer and/or cytotoxic activity, and diagnostic compounds typically include imaging probes.

In some embodiments, the compound of interest is a lipoparticle (such as a liposome or micelles) or a polymeric entity (such as albumin-based nanoparticles and polymer-based polymersomes) used as a carrier (or cargo) comprising, or encapsulating a diagnostic or therapeutic compound (Villaraza et al. 2010 Chem Rev., 110, 2921-2959). Hence, sdAbss are very convenient tools for delivering toxic cargos to cancer cells and are well-suited for chemical conjugation onto different nanoparticle formats.

The term “toxin,” “cytotoxin” or “cytotoxic compound” as used herein, refers to any agent that is detrimental to the growth and proliferation of cells and may act to reduce, inhibit, or destroy a cell or malignancy.

The term “anti-cancer compound” as used herein refers to any agent that can be used to treat a cell proliferative disorder such as cancer, including but not limited to, cytotoxic agents, chemotherapeutic agents, radioisotopes, targeted anti-cancer agents, immunotherapeutic agents (such as immunosuppressants or immune stimulators), and lytic peptides A therapeutic compound having anti-cancer or cytotoxic activity can be for example selected from a group consisting of a V-ATPase inhibitor, a pro-apoptotic agent, a Bcl2 inhibitor, an MCL1 inhibitor, a HSP90 inhibitor, an IAP inhibitor, an mTor inhibitor, a microtubule stabilizer, a microtubule destabilizer, an auristatin, a dolastatin, a maytansinoid, a MetAP (methionine aminopeptidase), an inhibitor of nuclear export of proteins CRM1, a DPPIV inhibitor, proteasome inhibitors, inhibitors of phosphoryl transfer reactions in mitochondria, a protein synthesis inhibitor, a kinase inhibitor, a CDK2 inhibitor, a CDK9 inhibitor, a kinesin inhibitor, an HDAC inhibitor, a DNA damaging agent, a DNA alkylating agent, a DNA intercalator, a DNA minor groove binder and a DHFR inhibitor.

In some embodiments, the single domain antibody is conjugated to a cytotoxic moiety. The cytotoxic moiety may, for example, be selected from the group consisting of taxol; cytochalasin B; gramicidin D; ethidium bromide; emetine; mitomycin; etoposide; tenoposide; vincristine; vinblastine; colchicin; doxorubicin; daunorubicin; dihydroxyanthracindione; a tubulin-inhibitor such as maytansine or an analog or derivative thereof, an antimitotic agent such as mo no methyl auristatin E or F or an analog or derivative thereof, dolastatin 10 or 15 or an analogue thereof, irinotecan or an analogue thereof, mitoxantrone; mithramycin; actinomycin D; 1-dehydrotestosterone; a glucocorticoid; procaine; tetracaine; lidocaine; propranolol; puromycin; calicheamicin or an analog or derivative thereof, an antimetabolite such as methotrexate, 6 mercaptopurine, 6 thioguanine, cytarabine, fludarabin, 5 fluorouracil, decarbazine, hydroxyurea, asparaginase, gemcitabine, or cladribine; an alkylating agent such as mechlorethamine, thioepa, chlorambucil, melphalan, carmustine (BSNU), lomustine (CCNU), cyclophosphamide, busulfan, dibromomannitol, streptozotocin, dacarbazine (DTIC), procarbazine, mitomycin C; a platinum derivative such as cisplatin or carboplatin; duocarmycin A, duocarmycin SA, rachelmycin (CC-1065), or an analog or derivative thereof, an antibiotic such as dactinomycin, bleomycin, daunorubicin, doxorubicin, idarubicin, mithramycin, mitomycin, mitoxantrone, plicamycin, anthramycin (AMC)); pyrrolo[2,1-c][1,4]-benzodiazepines (PDB); diphtheria toxin and related molecules such as diphtheria A chain and active fragments thereof and hybrid molecules, ricin toxin such as ricin A or a deglycosylated ricin A chain toxin, cholera toxin, a Shiga-like toxin such as SLT I, SLT II, SLT IIV, LT toxin, C3 toxin, Shiga toxin, pertussis toxin, tetanus toxin, soybean Bowman-Birk protease inhibitor, Pseudomonas exotoxin, alorin, saporin, modeccin, gelanin, abrin A chain, modeccin A chain, alpha-sarcin, Aleuritesfordii proteins, dianthin proteins, Phytolaccaamericana proteins such as PAPI, PAPII, and PAP-S, momordicacharantia inhibitor, curcin, crotin, sapaonariaofficinalis inhibitor, gelonin, mitogellin, restrictocin, phenomycin, and enomycin toxins; ribonuclease (R ase); DNase I, Staphylococcal enterotoxin A; pokeweed antiviral protein; diphtherin toxin; and Pseudomonas endotoxin.

In some embodiments, the single domain antibody is conjugated to an auristatin or a peptide analog, derivative or prodrug thereof. Auristatins have been shown to interfere with microtubule dynamics, GTP hydrolysis and nuclear and cellular division (Woyke et al (2001) Antimicrob. Agents and Chemother. 45(12): 3580-3584) and have anti-cancer (U.S. Pat. No. 5,663,149) and antifungal activity (Pettit et al, (1998) Antimicrob. Agents and Chemother. 42: 2961-2965). For example, auristatin E can be reacted with para-acetyl benzoic acid or benzoylvaleric acid to produce AEB and AEVB, respectively. Other typical auristatin derivatives include AFP, MMAF (monomethylauristatin F), and MMAE (monomethylauristatin E). Suitable auristatins and auristatinanalogs, derivatives and prodrugs, as well as suitable linkers for conjugation of auristatins to Abs, are described in, e.g., U.S. Pat. Nos. 5,635,483, 5,780,588 and 6,214,345 and in International patent application publications WO02088172, WO2004010957, WO2005081711, WO2005084390, WO2006132670, WO03026577, WO200700860, WO207011968 and WO205082023.

In some embodiments, the single domain antibody is conjugated to Mertansine (also called emtansine or DM1) or a peptide analog, derivative or prodrug thereof. Mertansine is a tubulin inhibitor, meaning that it inhibits the assembly of microtubules by binding to tubulin.

In some embodiments, the single domain antibody is conjugated to pyrrolo[2,1-c][1,4]-benzodiazepine (PDB) or an analog, derivative or prodrug thereof. Suitable PDBs and PDB derivatives, and related technologies are described in, e.g., Hartley J. A. et al, Cancer Res 2010; 70(17): 6849-6858; Antonow D. et al, Cancer J 2008; 14(3): 154-169; Howard P. W. et al, Bioorg Med ChemLett 2009; 19: 6463-6466 and Sagnou et al, Bioorg Med ChemLett 2000; 10(18): 2083-2086.

In some embodiments, the single domain antibody is conjugated to a cytotoxic moiety selected from the group consisting of an anthracycline, maytansine, calicheamicin, duocarmycin, rachelmycin (CC-1065), dolastatin 10, dolastatin 15, irinotecan, monomethylauristatin E, monomethylauristatin F, a PDB, or an analog, derivative, or prodrug of any thereof.

In some embodiments, the single domain antibody is conjugated to an anthracycline or an analog, derivative or prodrug thereof. In some embodiments, the single domain antibody is conjugated to maytansine or an analog, derivative or prodrug thereof. In some embodiments, the single domain antibody is conjugated to calicheamicin or an analog, derivative or prodrug thereof. In some embodiments, the single domain antibody is conjugated to duocarmycin or an analog, derivative or prodrug thereof. In some embodiments, the single domain antibody is conjugated to rachelmycin (CC-1065) or an analog, derivative or prodrug thereof. In some embodiments, the antibody is conjugated to dolastatin 10 or an analog, derivative or prodrug thereof. In some embodiments, the antibody is conjugated to dolastatin 15 or an analog, derivative or prodrug thereof. In some embodiments, the antibody is conjugated to monomethylauristatin E or an analog, derivative or prodrug thereof. In some embodiments, the single domain antibody is conjugated to monomethylauristatin F or an analog, derivative or prodrug thereof. In some embodiments, the antibody is conjugated to pyrrolo[2,1-c][1,4]-benzodiazepine or an analog, derivative or prodrug thereof. In some embodiments, the single domain antibody is conjugated to irinotecan or an analog, derivative or prodrug thereof.

In some embodiments, the sdAb is conjugated to a nucleic acid or nucleic acid-associated molecule. In one such embodiment, the conjugated nucleic acid is a cytotoxic ribonuclease (RNase) or deoxy-ribonuclease (e.g., DNase I), an antisense nucleic acid, an inhibitory RNA molecule (e.g., a siRNA molecule) or an immuno stimulatory nucleic acid (e.g., an immunostimulatoryCpG motif-containing DNA molecule). In some embodiments, the antibody is conjugated to an aptamer or a ribozyme.

In some embodiments, the sdAb is conjugated, e.g., as a fusion protein, to a lytic peptide such as CLIP, Magainin 2, mellitin, Cecropin and PI 8.

In some embodiments, the single domain antibody is conjugated to a cytokine, such as, e.g., IL-2, IL-4, IL-6, IL-7, IL-10, IL-12, IL-13, IL-15, IL-18, IL-23, IL-24, IL-27, IL-28a, IL-28b, IL-29, KGF, IFNa, IFN3, IFNy, GM-CSF, CD40L, Flt3 ligand, stem cell factor, ancestim, and TNFa.

In some embodiments, the single domain antibody is conjugated to a radioisotope or to a radioisotope-containing chelate. For example, the antibody can be conjugated to a chelator linker, e.g. DOTA, DTPA or tiuxetan, which allows for the antibody to be complexed with a radioisotope. The single domain antibody may also or alternatively comprise or be conjugated to one or more radiolabeled amino acids or other radiolabeled molecules Non-limiting examples of radioisotopes include ³H, ¹⁴C, ¹⁵N, ³⁵S, ⁹⁰Y, ″Tc, ¹²⁵I, ¹³¹I, ¹⁸⁶Re, ²¹³Bi, ²²⁵Ac and ²²⁷Th. For therapeutic purposes, a radioisotope emitting beta- or alpha-particle radiation can be used, e.g., 1311, 90Y, 211At, 212Bi, 67Cu, 186Re, 188Re, and 212Pb.

A diagnostic compound can be selected from an enzyme, a fluorophore, a NMR or MRI contrast agent, a radioisotope or a nanoparticle. For example, the diagnostic compound can be selected from the group consisting of:
an enzyme such as horseradish peroxidase, alkaline phosphatase, glucose-6-phosphatase or beta-galactosidase;
a fluorophore such as green fluorescent protein (GFP), blue fluorescent dyes excited at wavelengths in the ultraviolet (UV) part of the spectrum (e.g. AMCA (7-amino-4-methylcournarin-3-acetic acid); Alexa Fluor® 350), green fluorescent dyes excited by blue light (e.g. FITC, Cy2, Alexa Fluor® 488), red fluorescent dyes excited by green light (e.g. rhodamines, Texas Red, Cy3, Alexa Fluor dyes 546, 564 and 594), or dyes excited with far-red light (e.g. Cy5) to be visualized with electronic detectors (CCD cameras, photomultipliers);
a radioisotope such as 18F, nC, 13N, 150, 68Ga, 82Rb, 44Sc, 64Cu, 86Y, 89Zr, 124I, 152Tb that can be used for PET imaging or 67Ga, 81mKr, 99mTc, mIn, 123I, 125I, 3 Xe, 201T1, 155Tb, 195mPt that can be used for SPECT/scintigraphic studies, or 14C, 3H, 35S, 3 P, 125I that can be 211 212 75 76 131 1 1 1 used for autoradiography or in situ hybridisation, or At-, Bi-, Br-, Br-, I-, In, 177Lu-, 212Pb-, 186Re-, 188Re-, 153Sm-, OY that can be used to label the compounds;
a NMR or MRI contrast agent such as the paramagnetic agents gadolinium (Gd), dysprosium (Dy) and manganese (Mn), and the superparamagnetic agents based on iron oxide (such as MION, SPIO or USPIO) or iron platinum (SIPP), and X-nuclei such as 18F, 13C, 23Na, 170, 15N;
a nanoparticle such as gold nanoparticles (B. Van de Broek et al, ACSNano, Vol. 5, No. 6, 4319-4328, 2011) or quantum dots (A. Sukhanova et al, 2012 Nanomedicine, 8 516-525).

In a preferred embodiment, said diagnostic compound is a fluorophore, more preferably Alexa Fluor® 488, or a MRI contrast agent, more preferably gadolinium.

When the diagnostic agent is used for detection, it may comprise a radioactive atom for scintigraphic studies, for example 99Tc or 123I, or a spin label for nuclear magnetic resonance (NMR) imaging (also known as MRI), such as 13C, 9F, Fe, Gd, 123I, n lIn, Mn, 15N or 70.

The substance of interest according to the present disclosure may or may not permeate the mammal or human blood-brain barrier.

In some embodiments, when the compound of interest is a heterologous polypeptide, the single domain antibody of the present disclosure can be (alternatively, or in addition) fused to one or more heterologous polypeptide(s) to form a fusion protein (also named herein “fusion polypeptide” or “polypeptide”). A “fusion” or “chimeric” protein or polypeptide comprises a first amino acid sequence linked to a second amino acid sequence with which it is not naturally linked in nature. The amino acid sequences, which normally exist in separate proteins can be brought together in the fusion polypeptide. A fusion protein or polypeptide is created, for example, by chemical synthesis, or by creating and translating a polynucleotide in which the polypeptide regions are encoded in the desired relationship.

According to the present disclosure, the fusion protein can thus comprise at least one isolated humanized single domain antibody (hsbAb) as herein described that is fused either directly or via a spacer at its C-terminal end and/or at its N terminal end, notably fused at its C-terminal end to the N-terminal end of the heterologous polypeptide, and/or at its N-terminal end to the C-terminal end of the heterologous polypeptide. As used herein, the term “directly” means that the (first or last) amino acid at the terminal end (N or C-terminal end) of the humanized single domain antibody is fused to the (first or last) amino acid at the terminal end (N or C-terminal end) of the heterologous polypeptide. In other words, in this embodiment, the last amino acid of the C-terminal end of said sdAb is directly linked by a covalent bond to the first amino acid of the N-terminal end of said heterologous polypeptide, or the first amino acid of the N-terminal end of said sdAb is directly linked by a covalent bond to the last amino acid of the C-terminal end of said heterologous polypeptide. As used herein, the term “spacer” also called “linker” refers to a sequence of at least one amino acid that links the sdAb of the present disclosure to the heterologous polypeptide. Such a spacer may be useful to prevent steric hindrances. Examples of linkers disclosed in the present disclosure have the following sequences (Gly3-Ser)₄, (Gly3-Ser), Ser-Gly or (Ala-Ala-Ala).

In some embodiments, the polypeptide or protein, can be an enzyme, such as a reporter enzyme, albumin, or an immunoglobulin.

In some embodiments the compound of interest can be one or more polypeptides comprising another or the same antigen binding domain to form a multivalent binding compound. Notably, the compound of interest can be one or more single domain antibodies as herein disclosed or not. The resulting fusion protein, or polypeptide, that comprises two or more antigen binding domains, notably that comprises or essentially consists of two or more single domain antibodies are referred to herein as “multivalent” polypeptides or “multivalent” antigen binding compounds. In some embodiments, said fusion protein or polypeptide can comprise at least one single domain antibody with a first binding domain, as herein described, and at least one other binding domain (e.g. directed against the same or another epitope, antigen, or target, selected from proteins, polypeptides or small molecules), which is typically also a single domain antibody. “Multispecific” (fusion) polypeptide refers to a polypeptide comprising at least two different antigen binding domains (i.e. that target different epitope, antigen or target), in opposition to a polypeptide comprising similar antigen binding domains, notably comprising the same single domain antibodies (“monospecific” (fusion) polypeptide).

Thus, in some embodiments, a fusion protein as herein described may also provide at least a second antigen binding domain directed against any desired protein, polypeptide, antigen, antigenic determinant or epitope. Said binding domain can be directed against HER2, notably against the same or different HER2 epitope, or may be directed against any other epitope, antigen or target selected from polypeptides, proteins or small molecules.

A “bispecific” fusion protein of the present disclosure is a fusion polypeptide that comprises at least one single domain antibody as herein disclosed directed against a first antigen (i.e. HER2) and at least one further binding domain directed against a second HER2 epitope or antigen (i.e. different from HER2), whereas a “trispecific” polypeptide of the present disclosure is a polypeptide that comprises at least one single domain antibody as herein disclosed and directed against a first antigen (i.e. HER2), at least one further binding domain directed against a second HER2 epitope or antigen (i.e. different from HER2) and at least one further binding domain directed against a third HER2 epitope or antigen (i.e. different from both i.e. first and second antigen); etc.

Typically antigens other than HER2 can be selected from CD19, CD20, CD22, CD33, PSMA, PSCA, BCMA, CS1, GPC3, CSPG4, EGFR, HER3, CA125, CD123, 5T4, IL-13R, CD2, CD3, CD16 (FcγRIII), CD23, Li CAM, MUC16, ROR1, SLAMF7, cKit, CD38, CD53, CD71, CD74, CD92, CD100, CD123, CD138, CD146 (MUC18), CD148, CD150, CD200, CD261, CD262, CD362, ROR1, mesothelin, CD33/IL3Ra, c-Met, Glycolipid F77, EGFRvlll, MART-1, gp100, GD-2, O-GD2, NKp46 receptor, or presented antigens like NY-ESO-1 or MAGE A3, human telomerase reverse transcriptase (hTERT), survivin, cytochrome P450 1 B1 (CY1 B), Wilm's tumor gene 1 (WT1), livin, alphafetoprotein (AFP), carcinoembryonic antigen (CEA), mucin 16, MUC1, p53, cyclin, an immune checkpoint target or combinations thereof.

In some embodiment, at least one further antigen of the multi-specific fusion polypeptide comprises at least an immune cell antigen such as one or more T cell antigens, one or more macrophage antigens, one or more NK cell antigens, one or more neutrophil antigens, and/or one or more eosinophil antigens, as typically exemplifier for Bispecific T-cell or NK-cell engager molecules (see notably for BiTEs® Wolf E, Hofmeister R, Kufer P, Schlereth B, Baeuerle P A. “BiTEs: bispecific antibody constructs with unique anti-tumor activity”. Drug Discov Today. 2005 Sep. 15; 10(18):1237-44. Review). Amongst others for T cell antigens, CD2 and framework sequences of T-cell receptor α and β chains can be used, notably CD2 or CD3 and most particularly the c chain of the CD3 complex. For example, for NK cell antigens fragments from the FcγRIII and/or from the NKp46 receptor can be used.

Said multispecific polypeptide can be used in immune cell redirecting immune therapies on the same principle as for CAR therapies (see for illustrative review Ellwanger K, Reusch U, Fucek I, et al. Redirected optimized cell killing (ROCK®): A highly versatile multispecific fit-for-purpose antibody platform for engaging innate immunity. MAbs. 2019; 11(5):899-918).

In some embodiments, a further binding domain can be directed against a serum protein so that the half-life of the single domain antibody is increased. Typically, said serum protein is albumin.

In some embodiments, a further binding domain can be directed against a receptor on the vascular endothelium of the blood-brain barrier so that the single domain antibodies of the present disclosure would cross the blood-brain barrier. The targeted receptors include transferrin receptor, insulin receptor, IGF-I and IGF-II receptors, among others.

In some embodiments, the one or more further binding domain may comprise one or more parts, fragments, or domains of conventional chain antibodies (and in particular human antibodies) and/or of heavy chain antibodies. For example, a single domain antibody as herein defined may be linked to a conventional (typically human) VH or VL optionally via a linker sequence.

In some embodiments, the polypeptides, or fusion proteins of the present disclosure can comprise a single domain antibody of the present disclosure that is linked to an immunoglobulin domain. For example, the polypeptides, or fusion proteins comprise a single domain antibody of the present disclosure that is linked to an immunoglobulin or a portion or fragment thereof. For example, the polypeptide, or fusion protein comprises a single domain antibody of the present disclosure that is linked to an Fc domain (CH2-CH3), notably a human Fc region. Fc region from various mammals (typically from human or mouse antibodies) antibody subclasses can be used. Said Fc portion may be useful for increasing the half-life and even the production of the single domain antibody of the present disclosure. For example, the Fc portion can bind to serum proteins and thus increases the half-life on the single domain antibody.

In some embodiments, at least one single domain antibody may also be linked to one or more (typically human) Hinge and/or CHI, and/or CH2 and/or CH3 domains, optionally via a linker sequence. For instance, a single domain antibody linked to a suitable CHI domain could for example be used—together with suitable light chains—to generate antibody fragments/structures analogous to conventional Fab fragments or F(ab′)₂ fragments, but in which one or (in case of an F(ab′)₂ fragment) both of the conventional VH domains have been replaced by a single domain antibody as herein defined.

In some embodiments, one or more single domain antibodies of the present disclosure may be linked (optionally via a suitable linker or hinge region) to one or more constant domains (for example, 2 or 3 constant domains that can be used as part of/to form an Fc portion), to an Fc portion and/or to one or more antibody parts, fragments or domains that confer one or more effector functions to the polypeptide of the present disclosure and/or may confer the ability to bind to one or more Fc receptors. For example, for this purpose, and without being limited thereto, the one or more further amino acid sequences may comprise one or more CH2 and/or CH3 domains of an antibody, such as from a heavy chain antibody and more typically from a conventional human chain antibody; and/or may form and Fc region, for example from IgG (e.g. from IgGl, IgG2, IgG3 or IgG4), from IgE or from another human Ig such as IgA, IgD or IgM.

### Chimeric Antigen Receptors

The terms “Chimeric antigen receptor” or “CAR” or “CARs” as used herein refer to engineered receptors, which graft an antigen specificity onto cells (for example T cells such as naive T cells, central memory T cells, effector memory T cells or combination thereof) thus combining the antigen binding properties of the antigen binding domain with the lytic capacity and self-renewal of T cells. CARs are also known as artificial T cell receptors, chimeric T cell receptors or chimeric immunoreceptors. The term “antigen binding domain or “antigen-specific targeting domain” as used herein refers to the region of the CAR which targets and binds to specific antigens. When a CAR is expressed in a host cell, this domain forms the extracellular domain (ectodomain).

The CAR of the present disclosure comprises a molecule of the general formula:

sdAb(n)-[optionally a hinge-] transmembrane domain-intracellular signaling domain,
wherein n is 1 or more.

In some embodiments, n is at least 2, for example 2, 3, 4 or 5. The sdAb(n) form the antigen binding domain and is/are located at the extracellular side when expressed in a cell.

In some embodiments, a CAR as herein described preferably comprises at least two antigen binding compounds (typically a single domain antibody), and can therefore targets one or more antigens. The antigen binding domain of a CAR of the present disclosure can comprise at least two sdAbs that are both specific for HER2 (typically the human HER2 protein), thus providing a bivalent binding molecule. In some embodiments, the antigen binding domain comprises two or at least two VH single domain antibodies that are both specific for HER2 but that may bind to different epitopes. In other words, the antigen binding domain of a CAR as herein disclosed may comprise a first single domain antibody that binds to a first epitope of HER2 and a second single domain antibody that binds to a second epitope of HER2. The epitopes may be overlapping. Thus, the antigen binding domain is biparatopic.

In other embodiments, the antigen binding domain comprises two single domain antibodies that are both specific for HER2 and bind to the same epitope. In this embodiments at least 2 identical anti HER2 sdAbs as herein disclosed may be used.

In some embodiments, the antigen binding domain comprises an anti HER2 sdAb according to the present disclosure and optionally another antigen binding domain that is specific for another antigen, thus providing a bispecific antigen binding domain. In other words, the antigen binding domain comprises a first single domain antibody that binds to a first target consisting in HER2 and a second single domain antibody that binds to a second target. Thus, in certain embodiments, the present disclosure relates to bispecific CARs.

In preferred embodiments the sdAb comprises CDRs selected from:
a CDR1 of SEQ ID NO: 1; a CDR2 of SEQ ID NO:2 and a CDR3 of SEQ ID NO:3,
a CDR1 of SEQ ID NO:4; a CDR2 of SEQ ID NO:5 and a CDR3 of SEQ ID NO:6,
a CDR1 of SEQ ID NO: 10; a CDR2 of SEQ ID NO:11 and a CDR3 of SEQ ID NO:12,
a CDR1 of SEQ ID NO:13; a CDR2 of SEQ ID NO:14 and a CDR3 of SEQ ID NO:15, or
a CDR1 of SEQ ID NO:16; a CDR2 of SEQ ID NO:17 and a CDR3 of SEQ ID NO:18.
   or has a sequence selected from:
a sequence selected from the group consisting of SEQ ID NO: 23, SEQ ID NO:24, SEQ ID NO:26, SEQ ID NO:27, and SEQ ID NO:28;
a sequence having at least 90% identity with a sequence selected from the group consisting of SEQ ID NO: 23, SEQ ID NO:24, SEQ ID NO:26, SEQ ID NO:27, and SEQ ID NO:28;
a CDR1 of SEQ ID NO:1; a CDR2 of SEQ ID NO:2 and a CDR3 of SEQ ID NO:3 and further having one or more conservative amino acid modifications in one or more of these CDRs;
a CDR1 of SEQ ID NO:4; a CDR2 of SEQ ID NO:5 and a CDR3 of SEQ ID NO:6 and further having one or more conservative amino acid modifications in one or more of these CDRs.
a CDR1 of SEQ ID NO:10; a CDR2 of SEQ ID NO:11 and a CDR3 of SEQ ID NO:12 and further having one or more conservative amino acid modifications in one or more of these CDRs.
a CDR1 of SEQ ID NO:13; a CDR2 of SEQ ID NO:14 and a CDR3 of SEQ ID NO:15 and further having one or more conservative amino acid modifications in one or more of these CDRs; or
a CDR1 of SEQ ID NO:16; a CDR2 of SEQ ID NO:17 and a CDR3 of SEQ ID NO:18 and further having one or more conservative amino acid modifications in one or more of these CDRs.

In some embodiments, the CAR does not comprise a sdAb having the sequence of SEQ ID NO:25 or does not comprise the CDRs of SEQ ID NO:7-9.

As used herein, the term “bispecific CAR” or “bispecific antigen binding domain” thus refers to a polypeptide that has specificity for two targets including HER2. Accordingly, a bispecific binding molecule as described herein can selectively and specifically bind to a cell that expresses (or displays on its cell surface) HER2 and the second target.

In other embodiments, the binding molecule comprises more than two antigen-binding domains providing a multispecific binding molecule. A multispecific antigen-binding domain as described herein can thus in addition to binding HER2 bind one or more additional targets, i.e., a multispecific polypeptide can bind at least two, at least three, at least four, at least five, at least six, or more targets, wherein the multispecific polypeptide agent has at least two, at least, at least three, at least four, at least five, at least six, or more target binding sites respectively.

In some embodiments, additional antigens that can be bound by a multispecific CAR according to the present disclosure include tumor antigens. In some embodiments, the tumor antigens are associated with a hematologic malignancy or with a solid tumor. For example, a tumor antigen can be selected from the group consisting of PSMA, PSCA, BCMA, CS1, GPC3, CSPG4, EGFR, HER3, CA125, CD123, 5T4, IL-13R, CD2, CD3, CD16 (FcγRIII), CD23, L1 CAM, MUC16, ROR1, SLAMF7, cKit, CD19, CD20, CD22, CD33, CD38, CD53, CD71, CD74, CD92, CD100, CD123, CD138, CD146 (MUC18), CD148, CD150, CD200, CD261, CD262, CD362, ROR1, mesothelin, CD33/IL3Ra, c-Met, Glycolipid F77, EGFRvlll, MART-1, gp100, GD-2, O-GD2, NKp46 receptor, presented antigens like NY-ESO-1 or MAGE A3, human telomerase reverse transcriptase (hTERT), survivin, cytochrome P450 1 B1 (CY1 B), Wilm's tumor gene 1 (WT1), livin, alphafetoprotein (AFP), carcinoembryonic antigen (CEA), mucin 16, MUC1, p53, cyclin, and an immune checkpoint target or combinations thereof. However, a skilled person would understand that other tumor antigens are also targets within the scope of the present disclosure.

In addition to a binding domain as described in detail above, a CAR of the present disclosure further comprises a transmembrane domain. A “transmembrane domain” (TMD) as used herein refers to the region of the CAR which crosses the plasma membrane and is connected to the endoplasmic signaling domain and the antigen binding domain, in case of the latter optionally via a hinge. In one embodiment, the transmembrane domain of the CAR of the present disclosure is the transmembrane region of a transmembrane protein (for example Type I transmembrane proteins), an artificial hydrophobic sequence or a combination thereof. In some embodiments, the transmembrane domain comprises the CD8 domain, the CD3zeta domain, the CD28 transmembrane domain, the DAP10 transmembrane domain, the DAP 12 transmembrane domain or combination thereof. Other transmembrane domains will be apparent to those of skill in the art and may be used in connection with alternate embodiments of the present disclosure.

DAP10 and DAP12 are adapters that partner with most activating NKRs expressed in NK cells and all NKRs expressed in T cells (see Chen X, Bai F, Sokol L, et al. A critical role for DAP10 and DAP12 in CD8+ T cell-mediated tissue damage in large granular lymphocyte leukemia. Blood. 2009; 113(14):3226-3234).

In the immune system, DAP12 (DNAX-activation protein 12) is found in cells of the myeloid lineage, such as macrophages and granulocytes, where it associates, for instance, with the triggering receptor expressed on myeloid cell members (TREM) and MDL1 (myeloid DAP12-associating lectin 1/CLEC5A), both involved in inflammatory responses against pathogens like viruses and bacteria (for review, see Bakker A. B. et al., 1999. “Myeloid DAP12-associating lectin (MDL)-1 is a cell surface receptor involved in the activation of myeloid cells”. Proc. Natl. Acad. Sci. USA 96: 9792-9796.). DAP12 possesses a single cytoplasmic immunoreceptor tyrosine-based activation motif (ITAM; D/ExxYxxL/Ix6-12YxxL/I) and signals by activating Syk protein tyrosine kinase, phosphoinositide 3-kinase (PI3K), and extracellular signal-regulated kinase (ERK/MAPK). This signaling pathway results in granule mobilization, target cell lysis, and cytokine production.

The DAP12 protein (Ref SeqGene: NG_009304.1, Uniprot ref: O43914) comprises a minimal extracellular region, mainly consisting of a cysteine residue that permits the creation of disulfide-bonded homodimers of DAP12, and which have no ligand-binding capacity. Intracellularly, DAP12 has a single ITAM, which after tyrosine phosphorylation recruits and activates notably Syk and ZAP70 in NK cells

By DAP12 it is herein intended to mean the wild-type human protein, one of its wild-type orthologs or a functional variant thereof. In any case, the functional variant comprises at least an extracellular domain, a transmembrane domain and an intracellular domain. Furthermore, a functional variant of DAP12 according to the present disclosure also comprises at least the ITAM (immunoreceptor tyrosine-based activation motif) sequence. Preferably the human wild-type DAP12 protein is used. In a well-suited embodiment, the DAP12 signal peptide (corresponding to the first 21 amino terminal amino acids including the methionine) may be replaced by another signal peptide such as the CD8 signal peptide).

DAP10 (DNAX-activation protein 10) is a type I membrane protein of 93 amino acids (Gene bank ref: human DAP10 protein: AAD47911.1). It contains a short extracellular domain, a transmembrane domain and a short cytoplasmic domain. The DAP10 cytoplasmic domain comprises an YINM signaling motif which provides co-stimulatory signaling in conjunction with the ITAM-based TCR/CD3 complex in T cells.

By DAP10 it is herein intended to mean the wild-type human protein, one of its wild-type orthologs or a functional variant thereof. In any case, the functional variant comprises at least an extracellular domain, a transmembrane domain and an intracellular domain. Furthermore, a functional variant of DAP10 as herein disclosed also comprises at least the YxxM motif Preferably, the human wild-type DAP10 protein is used. In a well-suited embodiment, the DAP10 signal peptide (corresponding to the first 21 amino terminal amino acids including the methionine) may be replaced by another signal peptide such as the CD8 signal peptide).

By full DAP10 or DAP12 it is preferably intended the human DAP10 or 12 according to the included database references and including or not the signal peptide as mentioned above. In some embodiments the CAR of the present disclosure comprises a derivative of DAP10 or DAP12 as above described comprising an extracellular domain, a transmembrane domain and an intracellular domain and having at least 90% (typically at least 91, 92, 93, 94, 95, 96, 97, 98, 99%) identity with DAP 10 or DAP12.

Preferably the extracellular domain of the DAP10, DAP12, or of one of their functional variants is fused to the binding domain as previously defined. Typically said extracellular domain of the DAP10, DAP12, or of one of their functional variants is fused to an antibody such as a single-chain Fv antibody or a nanobody. In one alternative embodiment, said extracellular domain of the DAP10, DAP12, or of one of their functional variants is fused to a hinge fused to the binding domain. A hinge may be any linker amino acid sequence comprising 2 to 50 amino acids, such as a CD8 hinge.

A CAR of the present disclosure further comprises an intracellular signaling domain. An “intracellular signaling domain”, “cytoplasmic domain” or “endodomain” is the domain that transmits activation signals to T cells and directs the cell to perform its specialized function. Examples of domains that transduce the effector function signal and can be used according to the present disclosure include but are not limited to the ζ chain of the T-cell receptor complex or any of its homologs (e.g., η chain, FcsRIy and β chains, MB 1 (Iga) chain, B29 (Ig) chain, etc.), human CD3zeta chain, CD3 polypeptides (Δ, δ and ε), syk family tyrosine kinases (Syk, ZAP 70, etc.), src family tyrosine kinases (Lck, Fyn, Lyn, etc.) and intracellular domains from other molecules involved in T-cell transduction, such as CD2, CD5, OX40, CD28, DAP 10 and DAP12. Other intracellular signaling domains will be apparent to those of skill in the art and may be used in connection with alternate embodiments of the present disclosure. In some embodiments, the intracellular domain in notably selected from the intracellular domain of DAP10, DAP12, CD28, the human CD3zeta chain and combination thereof.

Typically, a CAR according to the present disclosure can comprise DAP 10 or DAP12 and further comprises a CD3-ζ chain and/or a CD28 activation or costimulation domain.

Preferably, the CAR comprises additional activation or co-stimulation domain(s) (or intracellular domain) comprising a fragment of at least 50, 60, 70, 80, 90, 100, 1 10, 120, 150, or 200 amino acids of at least one additional activation domain selected from CD3-ζ chain (also shortly named ζ) and the cytoplasmic domain of a costimulatory receptors CD28, 4-1 BB (CD137), OX40 (CD134), LAG3, TRIM, HVEM, ICOS, CD27, or CD40L. In various embodiments, the CAR comprises additional activation domain(s) comprising a fragment of at least 20, 30, 40, 50, 60, 70, 80, 90, 100, 1 10, 120, 150, or 200 amino acids that shares at least than 90%, preferably more than 95%, more preferably more than 99% identity with the amino acid sequence of the additional activation domain above mentioned.

In some embodiments, a CAR of the present disclosure further comprises one or more co-stimulatory domains to enhance CAR-T cell activity after antigen specific engagement. Inclusion of this domain in the CAR of the present disclosure enhances the proliferation, survival and/or development of memory cells. The co-stimulatory domain is located intracellular\hat{ }. The co-stimulatory domain is a functional signaling domain obtained from a protein selected form the following group: CD3zeta, CD28, CD137 (4-IBB), CD134 (OX40), Dap10, CD27, CD2, CD5, ICAM-1, LFA-1 (CD1 la/CD18), Lck, TNFR-I, TNFR-II, Fas, CD30, CD40, LAG3, TRIM, HVEM, ICOS, CD40L or combinations thereof. Other co-stimulatory domains (e.g., from other proteins) will be apparent to those of skill in the art. Multiple co-stimulatory domains can be included in a single CAR to recruit multiple signaling pathways. In one embodiment, the co-stimulatory domain is obtained from 4-1 BB. The term “4-1 BB” refers to a member of the TNFR superfamily with an amino acid sequence provided as GenBank Acc. No. AAA62478.2, or the equivalent residues from a non-human species, e.g., rodent (e.g. mouse or rat), monkey or ape. The term “4-1 BB costimulatory domain” refers to amino acid residues 214-255 of GenBank Acc. No. AAA62478.2, or the equivalent residues from a non-human species, e.g., mouse, rodent, monkey, ape and the like.

In some embodiments of the present disclosure, the CAR only comprises DAP10, DAP12 or a variant thereof in its intracellular domain.

Example of CAR designs are notably provided in Jaspers J E, Brentjens R J. “Development of CAR T cells designed to improve antitumor efficacy and safety” (Pharmacol Ther. 2017; 178:83-91).

The results included therein showed that HER2sdAb-DAP10-z and HER2sdAb-41-z based CARs exhibit high cytotoxicity against cancer cell lines. Alternatively, while showing less cytotoxicity sdAb-DAP12 based CARs may be useful as they are expected to reduce side effects.

In some embodiments, a CAR of the present disclosure further comprises a hinge or spacer region which connects the extracellular antigen binding domain and the transmembrane domain. This hinge or spacer region can be used to achieve different lengths and flexibility of the resulting CAR. Examples of a hinge or spacer region that can be used according to the present disclosure include, but are not limited to, Fc fragments of antibodies or fragments or derivatives thereof, hinge regions of antibodies, or fragments or derivatives thereof, CH2 regions of antibodies, CH3 regions of antibodies, artificial spacer sequences, for example peptide sequences, or combinations thereof. Other hinge or spacer regions will be apparent to those of skill in the art and may be used in connection with alternate embodiments of the present disclosure. In one embodiment, the hinge is an lgG4 hinge or a CD8A hinge.

In some embodiments, a CAR of the present disclosure further comprises a “linker domain” or “linker region” that connects different domains of the CAR. This domain includes an oligo- or polypeptide region from about 1 to 100 amino acids in length. Suitable linkers will be apparent to those of skill in the art and may be used in connection with alternate embodiments of the present disclosure.

In some embodiments, a CAR of the present disclosure further comprises a “leader sequence” typically in N terminal position. In some embodiment, the leader sequence is a for example the CD8A domain.

In some embodiments the CAR further comprises a signal peptide located at the N-terminus of the polypeptide.

Suitable CAR constructs as per the present disclosure are notably disclosed in WO2019077165, or in WO2012079000A1. Advantageously according to the present disclosure, the scFv binding domain(s) as described in those patent applications is/are replaced with one or more single domain antibody and comprise(s) at least one anti-HER2 single domain antibody as herein described.

In some embodiments the signal peptide of DAP10 or DAP12 may be replaced by another signal peptide. For example, it has been noticed that the replacement of the signal peptide of DAP10 or DAP12 with the CD8 improves the CAR expression.

A CAR of the present disclosure may further include a label, for example a label that facilitates imaging, such as a fluorescent label or other tag. This can, for example be used in methods for imaging tumor binding. The label may be conjugated to the antigen binding domain.

In some embodiments, the CAR may include a protein domain such as a SBP (streptavidin-binding peptide) domain in the C terminal region. The CARs described herein may be synthesized as single polypeptide chains. In this embodiment, the antigen-specific targeting regions are at the N-terminus, arranged in tandem and are separated by a linker peptide.

### Nucleic Acids, Vectors, Host Cells

The present disclosure also provides isolated nucleic acids encoding a single domain antibody or a variant therefore or a CAR as previously described and nucleic acid constructs comprising thereof. A nucleic acid according to the present disclosure may be obtained by well-known methods of recombinant DNA technology and/or of chemical DNA synthesis. Also within the scope of the present disclosure, are sequences with at least 60%, 70%, 80% or 90% sequence identity thereto.

The term “nucleic acid,” “polynucleotide,” or “nucleic acid molecule” refers to deoxyribonucleic acid (DNA) or ribonucleic acid (RNA), or a combination of a DNA or RNA. RNA includes in vitro transcribed RNA or synthetic RNA or an mRNA sequence encoding a CAR polypeptide as described herein. The nucleic acid may further comprise a suicide gene. The construct may be in the form of a plasmid, vector, transcription or expression cassette.

The present disclosure thus also provides a recombinant expression cassette comprising a nucleic acid according to the present disclosure under the control of a transcriptional promoter allowing the regulation of the transcription of said nucleic acid in a host cell. Said nucleic acid can also be linked to appropriate control sequences allowing the regulation of its translation in a host cell.

The present disclosure also provides a recombinant vector (e.g., a recombinant expression vector) comprising a nucleic acid according to the present disclosure. Advantageously, said recombinant vector is a recombinant expression vector comprising an expression cassette according to the present disclosure.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors”.

A vector according to the present disclosure is preferably a vector suitable for stable gene transfer and long-term gene expression into mammalian cells, such as by replication of the sequence of interest, expression of this sequence, maintaining of this sequence in extrachromosomal form, or else integration into the chromosomal material of the host. The recombinant vectors are constructed using standard recombinant DNA technology techniques and produced using conventional methods that are known in the art.

In some embodiments, a vector of the present disclosure is an integrating vector, such as an integrating viral vector, such as in particular a retrovirus or AAV vector. Preferably, the viral vector is a lentiviral vector, most preferably an integrating viral vector.

Within the context of the present disclosure, a “lentiviral vector” means a non-replicating non-pathogenic virus engineered for the delivery of genetic material into cells, and requiring lentiviral proteins (e.g., Gag, Pol, and/or Env) that are provided in trans. Indeed, the lentiviral vector lacks expression of functional Gag, Pol, and Env proteins. The lentivirus vector is advantageously a self-inactivating vector (SIN vector). The lentiviral vector comprises advantageously a central polypurine tract/DNA FLAP sequence (cPPT-FLAP), and/or insulator sequence (s) such as chicken beta-globin insulator sequence(s) to improve expression of the gene(s) of interest. The lentiviral vector is advantageously pseudotyped with another envelope protein, preferably another viral envelope protein, preferably the vesicular stomatis virus (VSV) glycoprotein. In some preferred embodiments, said lentiviral vector is a human immunodeficiency virus (HIV) vector.

Lentiviral vectors derive from lentiviruses, in particular human immunodeficiency virus (HIV-1 or HIV-2), simian immunodeficiency virus (SIV), equine infectious encephalitis virus (EIAV), caprine arthritis encephalitis virus (CAEV), bovine immunodeficiency virus (BIV) and feline immunodeficiency virus (FIV), which are modified to remove genetic determinants involved in pathogenicity and introduce new determinants useful for obtaining therapeutic effects.

The lentiviral vector may be present in the form of an RNA or DNA molecule, depending on the stage of production or development of said retroviral vectors. The lentiviral vector can be in the form of a recombinant DNA molecule, such as a plasmid, or in the form of a lentiviral vector particle (interchangeably named lentiviral particle in the context of the present disclosure), such as an RNA molecule(s) within a complex of lentiviral and other proteins.

Such vectors are based on the separation of the cis- and trans-acting sequences. In order to generate replication-defective vectors, the trans-acting sequences (e.g., gag, pol, tat, rev, and env genes) can be deleted and replaced by an expression cassette encoding a transgene.

Efficient integration and replication in non-dividing cells generally require the presence of two c/s-acting sequences at the center of the lentiviral genome, the central polypurine tract (cPPT) and the central termination sequence (CTS). These lead to the formation of a triple-stranded DNA structure called the central DNA “flap”, which acts as a signal for uncoating of the pre-integration complex at the nuclear pore and efficient importation of the expression cassette into the nucleus of non-dividing cells, such as dendritic cells. In one embodiment, the present disclosure encompasses a lentiviral vector comprising a central polypurine tract and central termination sequence referred to as cPPT/CTS sequence as described, in particular, in the European patent application EP 2 169 073.

Further sequences are usually present in cis, such as the long terminal repeats (LTRs) that are involved in integration of the vector proviral DNA sequence into a host cell genome. Vectors may be obtained by mutating the LTR sequences, for instance, in domain U3 of said LTR (AU3) (Miyoshi H et al, 1998, J Virol. 72(10):8150-7; Zufferey et al., 1998, J V/ro/72(12):9873-80). Preferably, the vector does not contain an enhancer. In one embodiment, the present disclosure encompasses a lentiviral vector comprising LTR sequences, preferably with a mutated U3 region (AU3) removing promoter and enhancer sequences in the 3′ LTR.

The packaging sequence Ψ (psi) can also be incorporated to help the encapsidation of the polynucleotide sequence into the vector particles (Kessler et al., 2007, Leukemia, 21 (9): 1859-74; Paschen et al., 2004, Cancer Immunol Immunother 12(6): 196-203). In one embodiment, the present disclosure encompasses a lentiviral vector comprising a lentiviral packaging sequence Ψ (psi).

Further additional functional sequences, such as a transport RNA-binding site or primer binding site (PBS) or a Woodchuck PostTranscriptional Regulatory Element (WPRE), can also be advantageously included in the lentiviral vector polynucleotide sequence of the present disclosure, to obtain a more stable expression of the transgene in vivo. can also be advantageously included in the lentiviral vector polynucleotide sequence of the present disclosure, to obtain a more stable expression of the transgene in vivo. In some embodiments, the present disclosure encompasses a lentiviral vector comprising a PBS. In some embodiments, the present disclosure encompasses a lentiviral vector comprising a WPRE and/or an IRES.

Thus, in a preferred embodiment, the lentiviral vector comprises at least one cPPT/CTS sequence, one Ψ sequence, one (preferably 2) LTR sequence, and an expression cassette including a transgene under the transcriptional control of a β2ηη or class I MHC promoter.

In some embodiments of the present disclosure, a vector (i.e. a recombinant transfer vector) of the present disclosure is an expression vector comprising appropriate means for expression of the hook fusion protein and/or the target fusion protein in a host cell.

Various promoters may be used to drive high expression of the nucleic acid sequence encoding the hook fusion protein and/or the target fusion protein. The promoter may be a tissue-specific, ubiquitous, constitutive or inducible promoter. Preferred promoters are notably functional in T cells and/or NK cells, preferably human T cells and human NK cells. In particular, preferred promoters are able to drive high expression the target fusion protein (notably a CAR as previously defined) from lentivectors in T cells or NK cells, preferably human T cells or NK T cells. For example, a promoter according to the present disclosure can be selected from phosphoglycerate kinase promoter (PGK), spleen focus-forming virus (SFFV) promoters, elongation factor-1 alpha (EF-1 alpha) promoter including the short form of said promoter (EFS), viral promoters such as cytomegalovirus (CMV) immediate early enhancer and promoter, retroviral 5′ and 3′ LTR promoters including hybrid LTR promoters, human ubiquitin promoter, MHC class I promoter, MHC class II promoter, and 32 microglobulin (β2m) promoter. The promoters are advantageously human promoters, i.e., promoters from human cells or human viruses such as spleen focus-forming virus (SFFV). Human ubiquitin promoter, MHC class I promoter, MHC class II promoter, and β2 microglobulin (β2m) promoter are more particular preferred. Preferably, the MHC class I promoter is an HLA-A2 promoter, an HLA-B7 promoter, an HLA-Cw5 promoter, an HLA-F, or an HLA-E promoter. In some embodiments the promoter is not a CMV promoter/enhancer, or is not a dectin-2 or MHCII promoter. Such promoters are well-known in the art and their sequences are available in sequence data base.

Typically, lentiviral particles refer to the extracellular infectious form of a virus composed of genetic material made from either DNA or RNA (most preferably single stranded RNA) surrounded by a protein coat, called the capsid, and in some cases an envelope of lipids that surrounds the capsid. Thus a lentiviral vector particle (or a lentiviral particle) comprises a lentiviral vector as previously defined in association with viral proteins. The vector is preferably an integrating vector.

The RNA sequences of the lentiviral particle can be obtained by transcription from a double-stranded DNA sequence inserted into a host cell genome (proviral vector DNA) or can be obtained from the transient expression of plasmid DNA (plasmid vector DNA) in a transformed host cell. Appropriate methods for designing and preparing lentiviral particles in particular for therapeutic application are well-known in the art and are for example described in Merten O W, Hebben M, Bovolenta C. Production of lentiviral vectors. Mol Ther Methods Clin Dev. 2016 Apr. 13; 3:16017.

Preferably the lentiviral particles have the capacity for integration. As such, they contain a functional integrase protein. Non-integrating vector particles have one or more mutations that eliminate most or all of the integrating capacity of the lentiviral vector particles. For, example, a non-integrating vector particle can contain mutation(s) in the integrase encoded by the lentiviral pol gene that cause a reduction in integrating capacity. In contrast, an integrating vector particle comprises a functional integrase protein that does not contain any mutations that eliminate most, or all of the integrating capacity of the lentiviral vector particles.

In some embodiments, the present disclosure encompasses a vector system comprising one or more vector comprising:
(a) a nucleic acid comprising a nucleic acid sequence encoding a chimeric antigen receptor as previously defined, and optionally
(b) a nucleic acid encoding another protein or polypeptide
wherein the nucleic acids (a) and (b) are located on the same or on separated vectors.

Preferred nucleic acids (a) have been described in the prior section.

When the vector system comprises more than one vector, typically two or more vectors, said vectors are typically of the same type (e.g.: a lentiviral vector). In the following sections the vector can also be intended as “the one or more vector” or “the vector system”. Preferably the present disclosure encompasses a lentiviral vector system and notably a lentiviral particle system.

According to the present disclosure, the vector can be an expression vector. The vector can be a plasmid vector.

In one embodiment of the present disclosure, the nucleic acid encoding the CAR and the other protein are inserted into separate vectors.

In another embodiment, the nucleic acid encoding the CAR and the other protein are inserted into the same vector.

In the later embodiment, each coding sequence (i.e. the nucleic acids encoding respectively the other protein or polypeptide and the CAR) can be inserted in a separate expression cassette. Each expression cassette therefore comprises the coding sequence (open reading frame or ORF) functionally linked to the regulatory sequences which allow the expression of the corresponding protein in the host cell, such as in particular promoter, promoter/enhancer, initiation codon (ATG), codon stop, transcription termination signal.

Alternatively, the proteins may also be expressed from a unique expression cassette using an Internal Ribosome Entry Site (IRES), or a self-cleaving 2A peptide inserted between the two coding sequences to allow simultaneous expression.

Nucleic acids encoding the proteins can be inserted in a single expression vector, said single vector comprising a bicistronic expression cassette. Vectors containing biscitronic expression cassette are well known in the art. Advantageously, bicistronic expression cassettes contain an Internal Ribosome Entry Site (IRES) that enables the expression of both fusion proteins from a single promoter. Suitable commercially available bicistronic vectors can include, but are not limited to plasmids of the pIRES (Clontech), pBud (Invitrogen) and Vitality (Stratagene) series. Preferably, the nucleic acid located upstream of the IRES sequence is operably-linked to a promoter. Preferably the nucleic acid encoding the hook protein is inserted upstream of the IRES sequence and the nucleic acid encoding the target fusion protein is inserted downstream of said TRES sequence to ensure that enough the hook fusion protein will be sufficiently expressed to retain every target fusion protein. In some embodiments multicistronic expression vectors may be used wherein more than one, typically at least two, nucleic acids encoding each a distinct hook and at least one nucleic acid encoding a target fusion protein are inserted.

A self-cleaving 2A peptide can also be used in replacement of IRES. Such strategy is highly advantageous because of its small size and high cleavage and translation efficacy between nucleic acid sequences upstream and downstream of the 2A peptide. Suitable 2A peptide according to the present disclosure are notably described in Kim J H, Lee S-R, Li L-H, et al. High Cleavage Efficiency of a 2A Peptide Derived from Porcine Teschovirus-1 in Human Cell Lines, Zebrafish and Mice. PLoS ONE. 2011; 6(4):e18556, but see also Liu, Z., O. Chen, J. B. J. Wall, M. Zheng, Y. Zhou, L. Wang, H. Ruth Vaseghi, L. Qian, and J. Liu. 2017. Systematic comparison of 2A peptides for cloning multi-genes in a polycistronic vector. Scientific reports. 7:2193. 2A peptides can be selected from FMDV 2A (abbreviated herein as F2A); equine rhinitis A virus (ERAV) 2A (E2A); porcine teschovirus-1 2A (P2A) and Thoseaasigna virus 2A (T2A). P2A or T2A peptide is preferred.

The present disclosure also encompasses a viral particle system, wherein the one or more viral particle comprises a viral vector, typically an integrating viral vector, as previously defined. Preferably, the viral vector is a lentiviral vector and the viral particle is a lentiviral particle. In one embodiment, the viral particle system comprises separated particles comprising a viral vector encoding respectively the hook protein and the CAR. In an alternative embodiment, the viral particle system comprises one particle comprising viral vector encoding both the hook fusion protein and the CAR as previously described. The nucleic acid sequence encoding the hook protein and the nucleic acid sequence encoding the CAR are preferably expressed from a unique expression cassette as defined above.

The present disclosure also provides a host cell containing a nucleic acid construct as herein disclosed, notably a recombinant expression cassette or a recombinant vector according to the present disclosure. The host cell is either a prokaryotic or eukaryotic host cell. The terms “host cell” refers to a cell into which exogenous nucleic acid has been introduced, including the progeny of such cells. Host cells include “transformants” and “transformed cells”, which include the primary transformed cell and progeny derived therefrom without regard to the number of passages. Progeny may not be completely identical in nucleic acid content to a parent cell but may contain mutations. Mutant progeny that have the same function or biological activity as screened or selected for in the originally transformed cell are included herein.

The present disclosure also provides a method for producing in a host cell as defined above a polypeptide, consisting or comprising a single domain antibody or a CAR as previously defined, comprising the steps of:
providing a host cell containing a nucleic acid construct, a recombinant expression cassette or a recombinant vector according to the present disclosure,
culturing said host cell,
and optionally purifying the single domain antibody or CAR of the present disclosure.

Methods for purifying polypeptides are well known in the art, such as chromatography (e.g., ion exchange chromatography, gel permeation chromatography and reversed phase chromatography).

The present disclosure also encompasses compositions comprising a nucleic acid construct as herein disclosed.

### Immune Cells and Method for Obtaining Thereof

The present disclosure also provides isolated cells, populations of cells, cell lines, or cell cultures, comprising a nucleic acid construct as previously described, notably vectors and more particularly a viral vector particle encoding at least one or more CAR as previously described. Preferably the vectors and/or lentiviral particles further comprise a nucleic acid sequence encoding a hook protein.

In one embodiment, the cell contains the vector and/or viral vector particle integrated into the cellular genome. In one embodiment, the cell contains the vector stably expressing the CAR. In one embodiment, the cell produces lentiviral vector particles encoding the CARs.

The cells are preferably mammalian cells, particularly human cells. Particularly preferred are human non-dividing cells. Preferably, the cells are immune cells, As used herein, the term “immune cells” includes cells that are of hematopoietic origin and that play a role in the immune response. Immune cells include lymphocytes, such as B cells and T cells, natural killer cells (NK cells), myeloid cells, such as monocytes, macrophages, eosinophils, mast cells, basophils, and granulocytes.

As used herein, the term “T cell” includes cells bearing a T cell receptor (TCR), T-cells according to the present disclosure can be selected from the group consisting of inflammatory T-lymphocytes, cytotoxic T-lymphocytes, regulatory T-lymphocytes, Mucosal-Associated Invariant T cells (MAIT), Yδ T cell, tumour infiltrating lymphocyte (TILs) or helper T-lymphocytes included both type 1 and 2 helper T cells and Th17 helper cells. In another embodiment, said cell can be derived from the group consisting of CD4+T-lymphocytes and CD8+T-lymphocytes.

Said immune cells may originate from a healthy donor or from a subject suffering from a cancer.

Immune cells can be extracted from blood or derived from stem cells. The stem cells can be adult stem cells, embryonic stem cells, more particularly non-human stem cells, cord blood stem cells, progenitor cells, bone marrow stem cells, induced pluripotent stem cells, totipotent stem cells or hematopoietic stem cells. Representative human cells are CD34+ cells.

T-cells can be obtained from a number of non-limiting sources, including peripheral blood mononuclear cells, bone marrow, lymph node tissue, cord blood, thymus tissue, tissue from a site of infection, ascites, pleural effusion, spleen tissue, and tumors. In certain embodiments, T-cells can be obtained from a unit of blood collected from a subject using any number of techniques known to the skilled person, such as FICOLL™ separation. In one embodiment, cells from the circulating blood of a subject are obtained by apheresis. In certain embodiments, T-cells are isolated from PBMCs. PBMCs may be isolated from buffy coats obtained by density gradient centrifugation of whole blood, for instance centrifugation through a LYMPHOPREP™ gradient, a PERCOLL™ gradient or a FICOLL™ gradient. T-cells may be isolated from PBMCs by depletion of the monocytes, for instance by using CD14 DYNABEADS®. In some embodiments, red blood cells may be lysed prior to the density gradient centrifugation.

In another embodiment, said cell can be derived from a healthy donor, from a subject diagnosed with cancer. The cell can be autologous or allogeneic.

In allogeneic immune cell therapy, immune cells are collected from healthy donors, rather than the patient. Typically, these are HLA matched to reduce the likelihood of graft vs. host disease. Alternatively, universal ‘off the shelf’ products that may not require HLA matching comprise modifications designed to reduce graft vs. host disease, such as disruption or removal of the TCRαβ receptor. See Graham et al., Cells. 2018 October; 7(10): 155 for a review. Because a single gene encodes the alpha chain (TRAC) rather than the two genes encoding the beta chain, the TRAC locus is a typical target for removing or disrupting TCRαβ receptor expression. Alternatively, inhibitors of TCRαβ signalling may be expressed, e.g. truncated forms of CD3ζ can act as a TCR inhibitory molecule. Disruption or removal of HLA class I molecules has also been employed. Typically, gene disruption may be achieved using gene editing techniques such as zinc-finger nucleases (ZFNs), transcription activator-like effector nucleases (TALENs) and clustered regularly interspaced short palindromic repeat (CRISPR)-Cas-associated nucleases can advantageously be used (see Li, H., Yang, Y, Hong, W. et al. Applications of genome editing technology in the targeted therapy of human diseases: mechanisms, advances and prospects. Sig Transduct Target Ther 5, 1 (2020)). For example, Torikai et al., Blood. 2013; 122:1341-1349 used ZFNs to knock out the HLA-A locus, while Ren et al., Clin. Cancer Res. 2017; 23:2255-2266 knocked out Beta-2 microglobulin (B2M), which is required for HLA class I expression. Ren et al. simultaneously knocked out TCRαβ, B2M and the immune-checkpoint PD1.

Generally, the immune cells are activated and expanded to be utilized in the adoptive cell therapy. The immune cells as herein disclosed can be expanded in vivo or ex vivo. The immune cells, in particular T-cells can be activated and expanded generally using methods known in the art. Generally, the T-cells are expanded by contact with a surface having attached thereto an agent that stimulates a CD3/TCR complex associated signal and a ligand that stimulates a co-stimulatory molecule on the surface of the T cells.

Typically, the immune cell is modified to express chimeric antigen receptor as herein disclosed. Expression of multiple tumor-specific targets may reduce the chance of antigen escape by mutating or reducing expression of the target antigen. As previously described the CARs of the present disclosure may be multispecific CARs (i.e. directed against more than one antigen, that is directed against HER2 and at least another antigen). In addition, or alternatively, an immune cell as herein described may express one or more CAR(s) as herein defined and at least another CAR targeting one or more other antigen(s).

Methods by which immune cells can be genetically modified by repressing the expression of specific molecules and/or to express a recombinant antigen receptor are well known in the art. A nucleic acid molecule encoding the antigen receptor may be introduced into the cell in the form of e.g. a vector (such as viral or nonviral DNA plasmid-based vectors) or any other suitable nucleic acid construct. Typically, in some embodiments non-viral vectors strategies can be preferred to avoid major disadvantages of viral-based delivery systems. For example, recombinant expression may be achieved using transposon-based expression such as typically the Sleeping Beauty (SB) transposon system (see Molecular reconstruction of Sleeping Beauty, a Tcl-like transposon from fish, and its transposition in human cells. Ivics Z, Hackett P B, Plasterk R H, Izsvik Z Cell. 1997 Nov. 14; 91(4):501-10 or for review Hackett P B, Largaespada D A, Cooper U. A transposon and transposase system for human application. Mol Ther. 2010; 18(4):674-683; and Aronovich E L, McIvor R S, Hackett P B. The Sleeping Beauty transposon system: a non-viral vector for gene therapy. Hum Mol Genet. 2011; 20(R1):R14-R20.) or PiggyBac transposon system (see Woodard L E, Wilson M H. piggyBac-ing models and new therapeutic strategies. Trends Biotechnol. 2015; 33(9):525-533; Ivics Z, Li M A, Mites L, et al. Transposon-mediated genome manipulation in vertebrates. Nat Methods. 2009; 6(6):415-422; Li X, Burnight E R, Cooney A L, et al. piggyBac transposase tools for genome engineering. Proc Natl Acad Sci USA. 2013; 110(25):E2279-E2287; and Zhao, Shuang et al. “PiggyBac transposon vectors: the tools of the human gene encoding.” Translational lung cancer research vol. 5,1 (2016): 120-5)). Typically also, genome editing techniques such as zinc-finger nucleases (ZFNs), transcription activator-like effector nucleases (TALENs) and clustered regularly interspaced short palindromic repeat (CRISPR)-Cas-associated nucleases can advantageously be used (Li, H., Yang, Y, Hong, W. et al. Applications of genome editing technology in the targeted therapy of human diseases: mechanisms, advances and prospects. Sig Transduct Target Ther 5, 1 (2020); but see for example regarding the CRISPR-Cas system the recent works of Miura, H., Quadros, R., Gurumurthy, C. et al. Easi-CRISPR for creating knock-in and conditional knockout mouse models using long ssDNA donors. Nat Protoc 13, 195-215 (2018); Hendel, A., Bak, R., Clark, J. et al. Chemically modified guide RNAs enhance CRISPR-Cas genome editing in human primary cells. Nat Biotechnol 33, 985-989 (2015); Roth, T. L., Puig-Saus, C., Yu, R. et al. Reprogramming human T cell function and specificity with non-viral genome targeting. Nature 559, 405-409 (2018). https://doi.org/10.1038/s41586-018-0326-5 or Eyquem, J. et al. Targeting a CAR to the TRAC locus with CRISPR/Cas9 enhances tumour rejection. Nature 543, 113-117 (2017)).

Vectors, and their required components, are well known in the art. Nucleic acid molecules encoding antigen receptors can be generated using any method known in the art, e.g. molecular cloning using PCR. Antigen receptor sequences can be modified using commonly used methods, such as site-directed mutagenesis.

In another aspect, the present disclosure relates to an ex vivo method for generating a population of cells for use in adaptive immunotherapy comprising transforming said cell with a CAR as herein described.

### Compositions and Kits of the Present Disclosure

The present disclosure also encompasses pharmaceutical compositions comprising one or more anti-HER2 single domain antibody(ies), CAR(s), nucleic acid construct encoding thereof and/or one or more isolated cell(s) or cell population(s) comprising a CAR as herein disclosed, alone or in combination with at least one other agent, such as a stabilizing compound, which may be administered in any sterile, biocompatible pharmaceutical carrier and optionally formulated with formulated with sterile pharmaceutically acceptable buffer(s), diluent(s), and/or excipient(s). Pharmaceutically acceptable carriers typically enhance or stabilize the composition, and/or can be used to facilitate preparation of the composition. Pharmaceutically acceptable carriers include solvents, dispersion media, coatings, antibacterial and antifungal agents, isotonic and absorption delaying agents, and the like that are physiologically compatible, and in some embodiments, pharmaceutically inert.

Administration of a pharmaceutical composition comprising sdAbs as herein disclosed can be accomplished orally or parenterally. Methods of parenteral delivery include topical, intra-arterial (directly to the tumor), intramuscular, spinal, subcutaneous, intramedullary, intrathecal, intraventricular, intravenous, intraperitoneal, or intranasal administration.

The genetically modified cells or pharmaceutical composition of the present disclosure can be administered by any convenient route, including parenteral administration. Parenteral administration includes, for example, intravenous, intramuscular, intraarterial, intraperitoneal, intranasal, rectal, intravesical, intradermal, topical or subcutaneous administration. Compositions can take the form of one or more dosage units.

Thus, in addition to the active ingredients, these pharmaceutical compositions may contain suitable pharmaceutically acceptable carriers comprising excipients and auxiliaries which facilitate processing of the active compounds into preparations which can be used pharmaceutically. Further details on techniques for formulation and administration may be found in the latest edition of Remington's Pharmaceutical Sciences (Ed. Maack Publishing Co, Easton, Pa.).

Depending on the route of administration, the single domain antibody or variant thereof, may be coated in a material to protect the compound from the action of acids and other natural conditions that may inactivate the compound.

The composition is typically sterile and preferably fluid. Proper fluidity can be maintained, for example, by use of coating such as lecithin, by maintenance of required particle size in the case of dispersion and by use of surfactants. In many cases, it is preferable to include isotonic agents, for example, sugars, polyalcohols such as mannitol or sorbitol, and sodium chloride in the composition. Long-term absorption of the injectable compositions can be brought about by including in the composition an agent which delays absorption, for example, aluminum monostearate or gelatin.

Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions and the like, for ingestion by the patient.

Pharmaceutical preparations for oral use can be obtained through combination of active compounds with solid excipient, optionally grinding a resulting mixture, and processing the mixture of granules, after adding suitable auxiliaries, if desired, to obtain tablets or dragee cores. Suitable excipients are carbohydrate or protein fillers such as sugars, including lactose, sucrose, mannitol, or sorbitol; starch from corn, wheat, rice, potato, or other plants; cellulose such as methyl, cellulose, hydroxypropylmethylcellulose, or sodium carboxymethylcellulose; and gums including arabic and tragacanth; and proteins such as gelatin and collagen. If desired, disintegrating or solubilizing agents may be added, such as the cross-linked polyvinyl pyrrolidone, agar, alginic acid, or a salt thereof, such as sodium alginate.

Dragee cores are provided with suitable coatings such as concentrated sugar solutions, which may also contain gum arabic, talc, polyvinylpyrrolidone, carbopol gel, polyethylene glycol and/or titanium dioxide, lacquer solutions, and suitable organic solvents or solvent mixtures. Dyestuffs or pigments may be added to the tablets or dragee coatings for product identification or to characterize the quantity of active compound, ie. dosage.

Pharmaceutical preparations that can be used orally include push-fit capsules made of gelatin, as well as soft, sealed capsules made of gelatin and a coating such as glycerol or sorbitol. Push-fit capsules can contain active ingredients mixed with a filler or binders such as lactose or starches, lubricants such as talc or magnesium stearate, and optionally, stabilizers. In soft capsules, the active compounds may be dissolved or suspended in suitable liquids, such as fatty oils, liquid paraffin, or liquid polyethylene glycol with or without stabilizers.

Pharmaceutical formulations for parenteral administration include aqueous solutions of active compounds. For injection, the pharmaceutical compositions of the present disclosure may be formulated in aqueous solutions, preferably in physiologically compatible buffers such as Hank's solution, Ringer's solution, or physiologically buffered saline. Aqueous injection suspensions may contain substances that increase viscosity of the suspension, such as sodium carboxymethyl cellulose, sorbitol, or dextran. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Optionally, the suspension may also contain suitable stabilizers or agents which increase the solubility of the compounds to allow for the preparation of highly concentrated solutions.

For topical or nasal administration, penetrants appropriate to the particular barrier to be permeated are used in the formulation. Such penetrants are generally known in the art.

Pharmaceutical compositions of the disclosure can be prepared in accordance with methods well known and routinely practiced in the art. See. e.g., Remington: The Science and Practice of Pharmacy, Mack Publishing Co., 20th ed., 2000; and Sustained and Controlled Release Drug Delivery Systems, J R. Robinson, ed., Marcel Dekker, Inc., New York, 1978. Pharmaceutical compositions are preferably manufactured under GMP conditions.

The amount of the pharmaceutical composition of the present disclosure that is effective/active in the treatment of a particular disorder or condition will depend on the nature of the disorder or condition, and can be determined by standard clinical techniques. In addition, in vitro or in vivo assays can optionally be employed to help identify optimal dosage ranges. The precise dose to be employed in the compositions will also depend on the route of administration, and the seriousness of the disease or disorder, and should be decided according to the judgment of the practitioner and each patient's circumstances.

The compositions as herein disclosed comprise an effective amount of a binding molecule of the present disclosure (e.g. a single domain antibody or variant thereof or a chimeric antigen receptor) such that a suitable dosage will be obtained. The correct dosage of the compounds will vary according to the particular formulation, the mode of application, and its particular site, host and the disease being treated. Other factors like age, body weight, sex, diet, time of administration, rate of excretion, condition of the host, drug combinations, reaction sensitivities and severity of the disease shall be taken into account. Administration can be carried out continuously or periodically within the maximum tolerated dose.

Typically, this amount is at least about 0.01% of a binding molecule of the present disclosure by weight of the composition. Preferred compositions of the present disclosure are prepared so that a parenteral dosage unit contains from about 0.01% to about 2% by weight of the binding molecule of the present disclosure.

For intravenous administration, the composition can comprise from about typically about 0.1 mg/kg to about 250 mg/kg of the animal's body weight, preferably, between about 0.1 mg/kg and about 20 mg/kg of the animal's body weight, and more preferably about 1 mg/kg to about 10 mg/kg of the animal's body weight.

The present compositions can take the form of suitable carriers, such aerosols, sprays, suspensions, or any other form suitable for use. Other examples of suitable pharmaceutical carriers are described in “Remington's Pharmaceutical Sciences” by E. W. Martin.

The pharmaceutical compositions as herein disclosed can be co-administered with other therapeutics, for example anti-cancer agents.

### Medical Uses

The present disclosure also relates to an anti-HER2 single domain antibody or variant thereof as herein described, a CAR directed against HER2 or variant thereof as herein described, a nucleic acid encoding said anti-HER2 single domain antibody or CAR, or to a cell, line or cell population comprising a CAR as described herein for use as a medicament in therapy, in particular, for use in the treatment of cancer, typically for cancer cell therapy in a subject in need thereof. In this embodiment, the cell as above defined can be an autologous cell (from the treated subject) or an allogenic cell.

The present disclosure also relates to an anti-HER2 single domain antibody or variant thereof as herein described, a CAR directed against HER2 or variant thereof as herein described, a nucleic acid encoding said anti-HER2 single domain antibody or CAR, or to a cell, line or cell population comprising said CAR as described herein in the manufacture of a medicament, notably for the treatment of cancer, such as for cell therapy of cancer.

The present disclosure also encompasses methods for the prevention and/or treatment of cancer, comprising administering to a subject to an anti-HER2 single domain antibody or variant thereof as herein described, a CAR directed against HER2 or variant thereof as herein described, a nucleic acid encoding said anti-HER2 single domain antibody or CAR, or a cell, line or to a cell population comprising a CAR as described herein, said method comprising administering, to a subject in need thereof, a pharmaceutically active amount of an anti-HER2 single domain antibody or variant thereof, a CAR, a cell, line or cell population comprising a CAR as described herein and/or of a pharmaceutical composition of the present disclosure. The method may additionally comprise the step of identifying a subject who has cancer.

The present disclosure also includes the use of one or more of to the anti-HER2 single domain antibodies or variants thereof, CARs directed against HER2 or variants thereof, nucleic acids encoding said anti-HER2 single domain antibodies or CARs, cell lines or cell population comprising a CAR as described herein in targeted immune therapy. For example, sdAbs of the present disclosure and in particular variants thereof in the form of multispecific polypeptides further targeting an immune cell antigen, and CAR expressing immune cells (notably CAR T cells) may be used in immune cell redirecting immune therapies.

In another aspect, the present disclosure relates to a method for stimulating a T cell-mediated immune response to a target cell population or tissue in a subject, the method comprising administering to a subject an effective amount of a cell or cell population that expresses a CAR directed against HER2 as herein described.

In another aspect, the present disclosure relates to a method of providing an anti-tumor immunity in a subject, the method comprising administering to the mammal an effective amount of a cell or cell population genetically modified to express a CAR directed against HER2 as herein described, thereby providing an anti-tumor immunity in the subject.

The present disclosure also relates to an anti-HER2 single domain antibody (including variants thereof), a CAR directed against HER2 as herein described, or a nucleic acid construct encoding said humanized anti-HER2 SdAb or CAR, or to an immune cell expressing said CAR, as previously defined, for use in adoptive cell or CAR-T cell therapy in a subject. Typically, the immune cell for use in the method of the present disclosure is a redirected T-cell, e.g. a redirected CD8+ and/or CD4+ T-cell.

In some embodiments, anti-HER2 single domain antibodies (including variants thereof), and CARs directed against HER2 as herein described, as well as nucleic acid constructs encoding them and cells comprising such CARs are useful for inhibiting tumor growth, inducing differentiation, reducing tumor volume, and/or reducing the tumorigenicity of a tumor. The methods of use can be in vitro, ex vivo, or in vivo methods.

In certain aspects, the subject is a human. In certain aspects, the subject has a tumor or has had a tumor removed. The subject can also be at risk of developing a cancer.

The cancer can be a solid cancer or a liquid tumor. Cancers that may treated by methods, uses and compositions described herein include, but are not limited to, cancer cells from the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; Sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

More specific cancers which can be treated and/or prevented according to the present disclosure include HER-mediated cancers. Typically, HER2-mediated cancers are cancers wherein HER2 is expressed or overexpressed. Typical cancers wherein HER2 is expressed and/or overexpressed include breast cancers, gastric or stomach cancers, salivary duct carcinoma, lung adenocarcinomas (such as non-small cell lung (NSCLC)), ovarian cancers, uterine cancers (such as uterine serous endometrial carcinoma), colon cancers, glioblastoma and pancreatic cancers.

In some embodiments, cancer treatment, and/or adoptive cell cancer therapy as above described are administered in combination with additional cancer therapies. In some embodiments, cancer treatment and/or adoptive cell cancer therapy as above described are administered in combination with targeted therapy, immunotherapy such as immune checkpoint therapy and immune checkpoint inhibitor, co-stimulatory antibodies, chemotherapy and/or radiotherapy.

Immune checkpoint therapy such as checkpoint inhibitors include, but are not limited to programmed death-1 (PD-1) inhibitors, programmed death ligand-1 (PD-L1) inhibitors, programmed death ligand-2 (PD-L2) inhibitors, lymphocyte-activation gene 3 (LAG3) inhibitors, T-cell immunoglobulin and mucin-domain containing protein 3 (TIM-3) inhibitors, T cell immunoreceptor with Ig and ITIM domains (TIGIT) inhibitors, B- and T-lymphocyte attenuator (BTLA) inhibitors, V-domain Ig suppressor of T-cell activation (VISTA) inhibitors, cytotoxic T-lymphocyte-associated protein 4 (CTLA4) inhibitors, Indoleamine 2,3-dioxygenase (IDO) inhibitors, killer immunoglobulin-like receptors (KIR) inhibitors, KIR2L3 inhibitors, KIR3DL2 inhibitors and carcinoembryonic antigen-related cell adhesion molecule 1 (CEACAM-1) inhibitors. In particular, checkpoint inhibitors include antibodies anti-PD1, anti-PD-L1, anti-CTLA-4, anti-TIM-3, anti-LAG3. Co-stimulatory antibodies deliver positive signals through immune-regulatory receptors including but not limited to ICOS, CD137, CD27, OX-40 and GITR.

Example of anti-PD1 antibodies include, but are not limited to, nivolumab, cemiplimab (REGN2810 or REGN-2810), tislelizumab (BGB-A317), tislelizumab, spartalizumab (PDR001 or PDR-001), ABBV-181, JNJ-63723283, BI 754091, MAG012, TSR-042, AGEN2034, pidilizumab, nivolumab (ONO-4538, BMS-936558, MDX1106, GTPL7335 or Opdivo), pembrolizumab (MK-3475, MK03475, lambrolizumab, SCH-900475 or Keytruda) and antibodies described in International patent applications WO2004004771, WO2004056875, WO2006121168, WO2008156712, WO2009014708, WO2009114335, WO2013043569 and WO2014047350.

Example of anti-PD-L1 antibodies include, but are not limited to, LY3300054, atezolizumab, durvalumab and avelumab.

Example of anti-CTLA-4 antibodies include, but are not limited to, ipilimumab (see, e.g., US patents U.S. Pat. Nos. 6,984,720 and 8,017,114), tremelimumab (see, e.g., U.S. Pat. Nos. 7,109,003 and 8,143,379), single chain anti-CTLA4 antibodies (see, e.g., International patent applications WO1997020574 and WO2007123737) and antibodies described in U.S. Pat. No. 8,491,895.

Example of anti-VISTA antibodies are described in US patent application US20130177557.

Example of inhibitors of the LAG3 receptor are described in US patent U.S. Pat. No. 5,773,578.

Example of KIR inhibitor is IPH4102 targeting KIR3DL2.

As used herein, the term “chemotherapy” has its general meaning in the art and refers to the treatment that consists in administering to the patient a chemotherapeutic agent. A chemotherapeutic entity as used herein refers to an entity which is destructive to a cell, that is the entity reduces the viability of the cell. The chemotherapeutic entity may be a cytotoxic drug. Chemotherapeutic agents include, but are not limited to alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall; dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, caminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxy doxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; methylhydrazine derivatives including N-methylhydrazine (MIH) and procarbazine; PSK polysaccharide complex); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel and doxetaxel; chlorambucil; gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-1 1); topoisomerase inhibitor RFS 2000; difluoromethylomithine (DMFO); retinoids such as retinoic acid; capecitabine; anthracyclines, nitrosoureas, antimetabolites, epipodophylotoxins, enzymes such as L-asparaginase; anthracenediones; hormones and antagonists including adrenocorticosteroid antagonists such as prednisone and equivalents, dexamethasone and aminoglutethimide; progestin such as hydroxyprogesterone caproate, medroxyprogesterone acetate and megestrol acetate; estrogen such as diethylstilbestrol and ethinyl estradiol equivalents; antiestrogen such as tamoxifen; androgens including testosterone propionate and fluoxymesterone/equivalents; antiandrogens such as flutamide, gonadotropin-releasing hormone analogs and leuprolide; and non-steroidal antiandrogens such as flutamide; biological response modifiers such as IFNa, IL-2, G-CSF and GM-CSF; and pharmaceutically acceptable salts, acids or derivatives of any of the above.

Suitable examples of radiation therapies include, but are not limited to external beam radiotherapy (such as superficial X-rays therapy, orthovoltage X-rays therapy, megavoltage X-rays therapy, radiosurgery, stereotactic radiation therapy, Fractionated stereotactic radiation therapy, cobalt therapy, electron therapy, fast neutron therapy, neutron-capture therapy, proton therapy, intensity modulated radiation therapy (IMRT), 3-dimensional conformal radiation therapy (3D-CRT) and the like); brachytherapy; unsealed source radiotherapy; tomotherapy; and the like. Gamma rays are another form of photons used in radiotherapy. Gamma rays are produced spontaneously as certain elements (such as radium, uranium, and cobalt 60) release radiation as they decompose, or decay. In some embodiments, radiotherapy may be proton radiotherapy or proton minibeam radiation therapy. Proton radiotherapy is an ultra-precise form of radiotherapy that uses proton beams (Prezado Y, Jouvion G, Guardiola C, Gonzalez W, Juchaux M, Bergs J, Nauraye C, Labiod D, De Marzi L, Pouzoulet F, Patriarca A, Dendale R. Tumor Control in RG2 Glioma-Bearing Rats: A Comparison Between Proton Minibeam Therapy and Standard Proton Therapy. Int J Radiat Oncol Biol Phys. 2019 Jun. 1; 104(2):266-271. doi: 10.1016/j.ijrobp.2019.01.080; Prezado Y, Jouvion G, Patriarca A, Nauraye C, Guardiola C, Juchaux M, Lamirault C, Labiod D, Jourdain L, Sebrie C, Dendale R, Gonzalez W, Pouzoulet F. Proton minibeam radiation therapy widens the therapeutic index for high-grade gliomas. Sci Rep. 2018 Nov. 7; 8(1):16479. doi: 10.1038/s41598-018-34796-8). Radiotherapy may also be FLASH radiotherapy (FLASH-RT) or FLASH proton irradiation. FLASH radiotherapy involves the ultra-fast delivery of radiation treatment at dose rates several orders of magnitude greater than those currently in routine clinical practice (ultra-high dose rate) (Favaudon V, Fouillade C, Vozenin M C. The radiotherapy FLASH to save healthy tissues. Med Sci (Paris) 2015; 31: 121-123. DOI: 10.1051/medsci/20153102002); Patriarca A., Fouillade C. M., Martin F., Pouzoulet F., Nauraye C., et al. Experimental set-up for FLASH proton irradiation of small animals using a clinical system. Int J Radiat Oncol Biol Phys, 102 (2018), pp. 619-626. doi: 10.1016/j.ijrobp.2018.06.403. Epub 2018 Jul. 11).

“In combination” may refer to administration of the additional therapy before, at the same time as or after administration of the T cell composition according to the present disclosure.

In addition, or as an alternative to the combination with checkpoint blockade, the T cell composition of the present disclosure may also be genetically modified to render them resistant to immune-checkpoints using gene-editing technologies including but not limited to TALEN and Crispr/Cas. Such methods are known in the art, see e.g. US20140120622. Gene editing technologies may be used to prevent the expression of immune checkpoints expressed by T cells (see the above listed checkpoint inhibitors) and more particularly but not limited to PD-1, Lag-3, Tim-3, TIGIT, BTLA CTLA-4 and combinations of these. The T cell as discussed here may be modified by any of these methods.

The T cell according to the present disclosure may also be genetically modified to express molecules increasing homing into tumors and or to deliver inflammatory mediators into the tumor microenvironment, including but not limited to cytokines, soluble immune-regulatory receptors and/or ligands.

Having thus described different embodiments of the present disclosure, it should be noted by those skilled in the art that the disclosures herein are exemplary only and that various other alternatives, adaptations, and modifications may be made within the scope of the present disclosure. Accordingly, the present disclosure is not limited to the specific embodiments as illustrated herein.

### Diagnostic Tool

Single domain antibodies (sdAbs) can aid in early diagnosis and cancer prevention by detecting or defining biomarkers. sdAbs can improve current mAb-based diagnostic techniques due to their high specificity. Furthermore, their high stability under extremes of temperature, pH, or ionic strength, ensures that the application still can occur under harsh conditions.

Typically, anti-HER sdAbs as per the present disclosure can be used in cell-based ELISA assays. To perform sandwich ELISA, both a capturing and detecting nanobody are used, preferably targeting different epitopes on the antigen.

The small size of nanobodies is highly advantageous especially in the field of molecular imaging as it enables rapid tumor accumulation and homogenous distribution as well as efficient blood clearance, contributing to high tumor-to-background ratios. Moreover, nanobodies can be easily conjugated to several kinds of imaging agents and their high specificity renders their use relatively safe. Single-photon emission computed tomography (SPECT) is based on 7-rays and sdAb of the present disclosure can thus linked to radionuclides such as ⁹⁹mTc, ¹⁷⁷Lu, ¹²³I and ¹¹¹In. On the other hand, the positron-emitting radioisotopes ⁶⁸Ga, ¹²⁴I or ⁸⁹Zr can be used for positron emission tomography (PET) purposes.

In some embodiments of the present disclosure, the anti-HER2 single domain antibodies as herein described are useful for detecting the presence of HER2 in a biological sample. The term “detecting” as used herein encompasses quantitative or qualitative detection. As used herein, the term “biological sample” is intended to include tissues, cells, biological fluids and isolates thereof, isolated from a patient, as well as tissues, cells and fluids present within a patient, or subject. In certain aspects, a biological sample comprises one or more cell(s) or tissue(s). In certain aspects, such tissues include normal and/or cancerous tissues that express HER2, notably that express HER2 at higher levels relative to other tissues or similar tissue from a control subject or from a control population of subjects.

Also included is a method of diagnosing a disorder associated with an increased expression of HER2, typically HER2-associated cancers or tumors. In certain aspects, the method comprises:
contacting a (tested) biological sample with an anti-HER2 single domain antibody of the present disclosure;
determining the level of expression (either quantitatively or qualitatively) of HER2 in said sample by detecting binding of said humanized anti-HER2 sdAb to HER2 expressed by the sample (typically cells); and
comparing the level of expression of HER2 in said sample with a reference value.
Typically wherein a higher level of expression of HER2 in the biological sample as compared reference value indicates the presence of a disease associated with increased expression of HER2. In certain aspects, the disease is a cell proliferative disorder, such as a cancer or a tumor, in particular a HER2-mediated cancer. In certain aspects, biological sample is obtained from an individual suspected of or having an HER2 associated disease.
Typically, the reference value can be the level of HER2 expression in a control tissue corresponding to the same type of tissue of the sample, and in particular in the corresponding control cell. In some embodiments, the reference value can be obtained from a control, or reference, sample. The control sample can be a sample from the corresponding normal tissue obtained from the same subject or patient as the tested sample, from a control healthy subject or from a control population of healthy subjects.

In one embodiment, an anti-HER2 sdAb as herein disclosed is used to select subjects eligible for therapy with an anti-HER2 treatment or therapy, typically, wherein HER2 is a biomarker for the selection of patients. The disclosure further provides for the use of an anti-HER2 sdAb in a method of diagnosing a subject suffering from a disorder associated with an increased HER2 expression (e.g., a cancer), the method comprising: determining the presence or expression level of HER2 in a sample obtained from the subject by contacting the sample with an anti-HER2 sdAb as herein described and detecting the presence of the bound sdAb. The anti-HER therapy is typically an anti-HER2 antibody or a variant thereof, typically an anti-HER2 sdAb as herein disclosed or a variant thereof, a multivalent binding compound or a chimeric antigen receptor as also disclosed herein.

In certain aspects, a method of diagnosis or detection, such as those described above, comprises detecting binding of an anti-HER2 single domain antibody expressed on the surface of a cell or in a membrane preparation obtained from a cell expressing HER2 on its surface. An exemplary assay for detecting binding of a humanized anti-HER2 sdAb to HER2 expressed on the surface of a cell is a “FACS” assay.

Certain other methods can be used to detect binding of humanized anti-HER2 sdAb as herein disclosed to HER2. Such methods include, but are not limited to, antigen-binding assays that are well known in the art, such as western blots, radioimmunoassays, ELISA (enzyme linked immunosorbent assay), “sandwich” immunoassays, immunoprecipitation assays, fluorescent immunoassays, protein A immunoassays, and immunohistochemistry (IHC). Advantageously in these embodiments humanized anti-HER2 sdAbs as herein disclosed are linked to a diagnostic compound, in particular a detectable label, as previously described.

In one embodiment, the present disclosure also provides an in vitro method for predicting the responsiveness of an individual suffering from a cancer to a treatment with an anti-cancer therapy. Typically, the anti-cancer therapy is an anti-HER2 therapy and comprises an anti-HER2 antibody or a variant thereof, notably an anti-HER2 sdAb as herein described (for example conjugated to a cytotoxic moiety), a multivalent binding compound or a chimeric GPC4 antigen receptor (CAR) as notably previously herein defined. The method comprises: determining the presence or expression level of HER2 in a (test) sample obtained from the subject by contacting the sample with an anti-HER2 sdAb, as herein disclosed and detecting the presence of the bound sdAb, wherein the presence or expression level of HER2 in the tested sample indicates that the subject is more likely to respond to treatment with the anti-cancer therapy. Optionally, the expression level of HER2 can be quantified and compared to a reference value as previously defined. The reference value is typically a threshold value, wherein a HER2 expression level in the tested sample above the threshold value means that the subject is more likely to respond to treatment with the anti-cancer therapy.

In the following, the invention will be illustrated by means of the following examples and figures.

TABLE 2
Sequences of the present disclosure
CDRs are numbered according to
the IMGT nomenclature.

| Description | | SEQ | |
|---|---|---|---|
| of the | | ID | |
| sequence | type | NO: | sequence |
| CDR1 | aa | 1 | ATSNISN |
| sdAb nº1 | | | |
| CDR2 | aa | 2 | RAESRPL |
| sdAb nº1 | | | |
| CDR3 | aa | 3 | YMPLVRHKA |
| sdAb nº1 | | | |
| CDR1 | aa | 4 | DSYNESS |
| sdAb nº2 | | | |
| CDR2 | aa | 5 | ARGNHPL |
| sdAb nº2 | | | |
| CDR3 | aa | 6 | SMPMPKWKK |
| sdAb nº2 | | | |
| CDR1 | aa | 7 | RYYEQSI |
| sdAb nº3 | | | |
| CDR2 | aa | 8 | EYGGWQH |
| sdAb nº3 | | | |
| CDR3 | aa | 9 | IRHQNQSMM |
| sdAb nº3 | | | |
| CDR1 | aa | 10 | YTSRESG |
| sdAb nº4 | | | |
| CDR2 | aa | 11 | RDQDGIS |
| sdAb nº4 | | | |
| CDR3 | aa | 12 | MIPLTRNAK |
| sdAb nº4 | | | |
| CDR1 | aa | 13 | YTFSEET |
| sdAb nº5 | | | |
| CDR2 | aa | 14 | WNHTFFE |
| sdAb nº5 | | | |
| CDR3 | aa | 15 | VTPLPPNKA |
| sdAb nº5 | | | |
| CDR1 | aa | 16 | RGYTGTT |
| sdAb nº6 | | | |
| CDR2 | aa | 17 | WTSNQTS |
| sdAb nº6 | | | |
| CDR3 | aa | 18 | MMPLPRWKG |
| sdAb nº6 | | | |
| FR1 | aa | 19 | MAEVQLQASGGGFVQPGGSL |
| | | | RLSCAASG |
| FR2 | aa | 20 | MGWFRQAPGKEREFVSAIS |
| FR3 | aa | 21 | YYADSVKGRFTISRDNSKNT |
| | | | VYLQMNSLRAEDTATYYCA |
| FR4 | aa | 22 | YWGQGTQVTVSS |
| SdAb nº1 | aa | 23 | MAEVQLQASGGGFVQPGGSL |
| | | | RLSCAASGATSNISNMGWFR |
| | | | QAPGKEREFVSAISRAESRP |
| | | | LYYADSVKGRFTISRDNSKN |
| | | | TVYLQMNSLRAEDTATYYCA |
| | | | YMPLVRHKAYWGQGTQVTVS |
| | | | S |
| sdAb nº2 | aa | 24 | MAEVQLQASGGGFVQPGGSL |
| | | | RLSCAASGDSYNESSMGWFR |
| | | | QAPGKEREFVSAISARGNHP |
| | | | LYYADSVKGRFTISRDNSKN |
| | | | TVYLQMNSLRAEDTATYYCA |
| | | | SMPMPKWKKYWGQGTQVTVS |
| | | | S |
| sdAb nº3 | aa | 25 | MAEVQLQASGGGFVQPGGSL |
| | | | RLSCAASGRYYEQSIMGWFR |
| | | | QAPGKEREFVSAISEYGGWQ |
| | | | HYYADSVKGRFTISRDNSKN |
| | | | TVYLQMNSLRAEDTATYYCA |
| | | | IRHQNQSMMYWGQGTQVTVS |
| | | | S |
| SdAb nº4 | aa | 26 | MAEVQLQASGGGFVQPGGSL |
| | | | RLSCAASGYTSRESGMGWFR |
| | | | QAPGKEREFVSAISRDQDGI |
| | | | SYYADSVKGRFTISRDNSKN |
| | | | TVYLQMNSLRAEDTATYYCA |
| | | | MIPLTRNAKYWGQGTQVTVS |
| | | | S |
| sdAb nº5 | aa | 27 | MAEVQLQASGGGFVQPGGSL |
| | | | RLSCAASGYTFSEETMGWFR |
| | | | QAPGKEREFVSAISWNHTFF |
| | | | EYYADSVKGRFTISRDNSKN |
| | | | TVYLQMNSLRAEDTATYYCA |
| | | | VTPLPPNKAYWGQGTQVTVS |
| | | | S |
| sdAb nº6 | aa | 28 | MAEVQLQASGGGFVQPGGSL |
| | | | RLSCAASGRGYTGTTMGWFR |
| | | | QAPGKEREFVSAISWTSNQT |
| | | | SYYADSVKGRFTISRDNSKN |
| | | | TVYLQMNSLRAEDTATYYCA |
| | | | MMPLPRWKGYWGQGTQVTVS |
| | | | S |
| Human | aa | 29 | MELAALCRWGLLLALLPPGA |
| HER2 | | | ASTQVCTGTDMKLRLPASPE |
| | | | THLDMLRHLYQGCQVVQGNL |
| | | | ELTYLPTNASLSFLQDIQEV |
| | | | QGYVLIAHNQVRQVPLQRLR |
| | | | IVRGTQLFEDNYALAVLDNG |
| | | | DPLNNTTPVTGASPGGLREL |
| | | | QLRSLTEILKGGVLIQRNPQ |
| | | | LCYQDTILWKDIFHKNNQLA |
| | | | LTLIDTNRSRACHPCSPMCK |
| | | | GSRCWGESSEDCQSLTRTVC |
| | | | AGGCARCKGPLPTDCCHEQC |
| | | | AAGCTGPKHSDCLACLHFNH |
| | | | SGICELHCPALVTYNTDTFE |
| | | | SMPNPEGRYTFGASCVTACP |
| | | | YNYLSTDVGSCTLVCPLHNQ |
| | | | EVTAEDGTQRCEKCSKPCAR |
| | | | VCYGLGMEHLREVRAVTSAN |
| | | | IQEFAGCKKIFGSLAFLPES |
| | | | FDGDPASNTAPLQPEQLQVF |
| | | | ETLEEITGYLYISAWPDSLP |
| | | | DLSVFQNLQVIRGRILHNGA |
| | | | YSLTLQGLGISWLGLRSLRE |
| | | | LGSGLALIHHNTHLCFVHTV |
| | | | PWDQLFRNPHQALLHTANRP |
| | | | EDECVGEGLACHQLCARGHC |
| | | | WGPGPTQCVNCSQFLRGQEC |
| | | | VEECRVLQGLPREYVNARHC |
| | | | LPCHPECQPQNGSVTCFGPE |
| | | | ADQCVACAHYKDPPFCVARC |
| | | | PSGVKPDLSYMPIWKFPDEE |
| | | | GACQPCPINCTHSCVDLDDK |
| | | | GCPAEQRASPLTSIISAVVG |
| | | | ILLVVVLGVVFGILIKRRQQ |
| | | | KIRKYTMRRLLQETELVEP |
| | | | LTPSGAMPNQAQMRILKETE |
| | | | LRKVKVLGSGAFGTVYKGIW |
| | | | IPDGENVKIPVAIKVLRENT |
| | | | SPKANKEILDEAYVMAGVGS |
| | | | PYVSRLLGICLTSTVQLVTQ |
| | | | LMPYGCLLDHVRENRGRLGS |
| | | | QDLLNWCMQIAKGMSYLEDV |
| | | | RLVHRDLAARNVLVKSPNHV |
| | | | KITDFGLARLLDIDETEYHA |
| | | | DGGKVPIKWMALESILRRRF |
| | | | THQSDVWSYGVTVWELMTFG |
| | | | AKPYDGIPAREIPDLLEKGE |
| | | | RLPQPPICTIDVYMIMVKCW |
| | | | MIDSECRPRFRELVSEFSRM |
| | | | ARDPQRFVVIQNEDLGPASP |
| | | | LDSTFYRSLLEDDDMGDLVD |
| | | | AEEYLVPQQGFFCPDPAPG |
| | | | AGGMVHHRHRSSSTRSGGGD |
| | | | LTLGLEPSEEEAPRSPLAPS |
| | | | EGAGSDVFDGDLGMGAAKGL |
| | | | QSLPTHDPSPLQRYSEDPTV |
| | | | PLPSETDGYVAPLTCSPQPE |
| | | | YVNQPDVRPQPPSPREGPLP |
| | | | AARPAGATLERPKTLSPGKN |
| | | | GVVKDVFAFGGAVENPEYLT |
| | | | PQGGAAPQPHPPPAFSPAFD |
| | | | NLYYWDQDPPERGAPPSTFK |
| | | | GTPTAENPEYLGLDVPV |
| SdAb nº1 | nt | 30 | ATGGCGGAAGTGCAGCTGCA |
| | | | GGCTTCCGGGGGAGGATTTG |
| | | | TGCAGCCGGGGGGGTCATTG |
| | | | CGACTGAGCTGCGCCGCATC |
| | | | CGGAGCAACATCAAACATCA |
| | | | GTAACATGGGCTGGTTTCGT |
| | | | CAGGCCCCTGGCAAGGAGAG |
| | | | AGAGTTCGTTTCCGCCATCT |
| | | | CCCGTGCAGAATCGCGTCCT |
| | | | CTGTATTACGCTGACAGCGT |
| | | | AAAGGGAAGATTTACAATTA |
| | | | GCCGGGATAACTCCAAAAAC |
| | | | ACGGTCTATCTCCAGATGAA |
| | | | CAGCCTCAGGGCCGAGGACA |
| | | | CAGCTACGTATTACTGTGCA |
| | | | TATATGCCTCTGGTTCGTCA |
| | | | CAAGGCATACTGGGGACAGG |
| | | | GGACGCAGGTAACTGTGAGT |
| | | | AGC |
| sdAb nº2 | nt | 31 | ATGGCGGAAGTGCAGCTGCA |
| | | | GGCTTCCGGGGGAGGATTTG |
| | | | TGCAGCCGGGGGGGTCATTG |
| | | | CGACTGAGCTGCGCCGCATC |
| | | | CGGAGATTCCTACAACGAGA |
| | | | GTTCTATGGGCTGGTTTCGT |
| | | | CAGGCCCCTGGCAAGGAGAG |
| | | | AGAGTTCGTTTCCGCCATCT |
| | | | CGGCACGTGGTAACCATCCT |
| | | | CTGTATTACGCTGACAGCGT |
| | | | AAAGGGAAGATTTACAATTA |
| | | | GCCGGGATAACTCCAAAAAC |
| | | | ACGGTCTATCTCCAGATGAA |
| | | | CAGCCTCAGGGCCGAGGACA |
| | | | CAGCTACGTATTACTGTGCA |
| | | | TCGATGCCTATGCCTAAGTG |
| | | | GAAGAAGTACTGGGGACAGG |
| | | | GGACGCAGGTAACTGTGAGT |
| | | | AGC |
| sdAb n°3 | nt | 32 | ATGGCGGAAGTGCAGCTGCA |
| | | | GGCTTCCGGGGGAGGATTTG |
| | | | TGCAGCCGGGGTGGTCATTG |
| | | | CGACTGAGCTGCGCCGCATC |
| | | | CGGACGTTACTACGAGCAGA |
| | | | GTATTATGGGCTGGTTTCGT |
| | | | CAGGCCCCTGGCAAGGAGAG |
| | | | AGAGTTCGTTTCCGCCATCT |
| | | | CCGAATATGGTGGTTGGCAG |
| | | | CATTACTACGCTGACAGCGT |
| | | | AAAGGGAAGATTTACAATTA |
| | | | GCCGGGATAACTCCAAAAAC |
| | | | ACGGTCTATCTCCAGATGAA |
| | | | CAGCCTCAGGGCCGAGGACA |
| | | | CAGCTACGTATTACTGTGCA |
| | | | ATTCGTCACCAGAACCAGTC |
| | | | GATGATGTATTGGGGACAGG |
| | | | GGACGCAGGTAACTGTGAGT |
| | | | AGC |
| SdAb nº4 | nt | 33 | ATGGCGGAAGTGCAGCTGCA |
| | | | GGCTTCCGGGGGAGGATTTG |
| | | | TGCAGCCGGGGGGGTCATTG |
| | | | CGACTGAGCTGCGCCGCATC |
| | | | CGGCTATACTTCACGTGAGT |
| | | | CCGGTATGGGCTGGTTTCGT |
| | | | CAGGCCCCTGGCAAGGAGAG |
| | | | AGAGTTCGTTTCCGCCATCT |
| | | | CCCGTGACCAAGACGGTATT |
| | | | TCGTACTACGCTGACAGCGT |
| | | | AAAGGGAAGATTTACAATTA |
| | | | GCCGGGATAACTCCAAAAAC |
| | | | ACGGTCTATCTCCAGATGAA |
| | | | CAGCCTCAGGGCCGAGGACA |
| | | | CAGCTACGTATTACTGTGCC |
| | | | ATGATTCCTCTGACACGTAA |
| | | | CGCAAAGTATTGGGGACAGG |
| | | | GGACGCAGGTAACTGTGAGT |
| | | | AGC |
| sdAb nº5 | nt | 34 | ATGGCGGAAGTGCAGCTGCA |
| | | | GGCTTCCGGGGGAGGATTTG |
| | | | TGCAGCCGGGGGGGTCATTG |
| | | | CGACTGAGCTGCGCCGCATC |
| | | | CGGCTATACTTTCAGTGAGG |
| | | | AGACAATGGGCTGGTTTCGT |
| | | | CAGGCCCCTGGCAAGGAGAG |
| | | | AGAGTTCGTTTCCGCCATCT |
| | | | CGTGGAACCATACATTTTTT |
| | | | GAGTATTACGCTGACAGCGT |
| | | | AAAGGGAAGATTTACAATTA |
| | | | GCCGGGATAACTCCAAAAAC |
| | | | ACGGTCTATCTCCAGATGAA |
| | | | CAGCCTCAGGGCCGAGGACA |
| | | | CAGCTACGTATTACTGTGCC |
| | | | GTTACACCTCTGCCTCCTAA |
| | | | CAAGGCATATTGGGGACAGG |
| | | | GGACGCAGGTAACTGTGAGT |
| | | | AGC |
| sdAb nº6 | nt | 35 | ATGGCGGAAGTGCAGCTGCA |
| | | | GGCTTCCGGGGGAGGATTTG |
| | | | TGCAGCCGGGGGGGTCATTG |
| | | | CGACTGAGCTGCGCCGCATC |
| | | | CGGACGTGGATATACAGGTA |
| | | | CTACAATGGGCTGGTTTCGT |
| | | | CAGGCCCCTGGCAAGGAGAG |
| | | | AGAGTTCGTTTCCGCCATCT |
| | | | CGTGGACATCGAACCAGACA |
| | | | TCGTACTACGCTGACAGCGT |
| | | | AAAGGGAAGATTTACAATTA |
| | | | GCCGGGATAACTCCAAAAAC |
| | | | ACGGTCTATCTCCAGATGAA |
| | | | CAGCCTCAGGGCCGAGGACA |
| | | | CAGCTACGTATTACTGTGCA |
| | | | ATGATGCCTCTGCCTCGTTG |
| | | | GAAGGGTTATTGGGGACAGG |
| | | | GGACGCAGGTAACTGTGAGT |
| | | | AGC |
| anti-HER2 | nt | 36 | atggccttaccagtgaccgc |
| hsdAb1 | | | cttgctcctgccgctggcct |
| 41BB-z | | | tgctgctccacgccgccagg |
| (myc tag) | | | cattctagagcggaagtgca |
| | | | gctgcaggcttccgggggag |
| | | | gatttgtgcagccggggggg |
| | | | tcattgcgactgagctgcgc |
| | | | cgcatccggagcaacatcaa |
| | | | acatcagtaacatgggctgg |
| | | | tttcgtcaggcccctggcaa |
| | | | ggagagagagttcgtttccg |
| | | | ccatctcccgtgcagaatcg |
| | | | cgtcctctgtattacgctga |
| | | | cagcgtaaagggaagattta |
| | | | caattagccgggataactcc |
| | | | aaaaacacggtctatctcca |
| | | | gatgaacagcctcagggccg |
| | | | aggacacagctacgtattac |
| | | | tgtgcatatatgcctctggt |
| | | | tcgcacaaggcatactgggg |
| | | | acaggggacgcaggtaactg |
| | | | tgagtagccctgcaggagag |
| | | | cagaagctgatctcagagga |
| | | | ggacctgcatatgaccacga |
| | | | cgccagcgccgcgaccacca |
| | | | acaccggcgcccaccatcgc |
| | | | gtcgcagcccctgtccctgc |
| | | | gcccagaggcgtgccggcca |
| | | | gcggcggggggcgcagtgca |
| | | | cacgagggggctggacttcg |
| | | | cctgtgatatctacatctgg |
| | | | gcgcccttggccgggacttg |
| | | | tggggtccttctcctgtcac |
| | | | tggttatcaccctttactgc |
| | | | aaacggggcagaaagaaact |
| | | | cctgtatatattcaaacaac |
| | | | catttatgagaccagtacaa |
| | | | actactcaagaggaagatgg |
| | | | ctgtagctgccgatttccag |
| | | | aagaagaagaaggaggatgt |
| | | | gaactgagagtgaagttcag |
| | | | caggagcgcagacgcccccg |
| | | | cgtacaagcagggccagaac |
| | | | cagctctataacgagctcaa |
| | | | tctaggacgaagagaggagt |
| | | | acgatgttttggacaagaga |
| | | | cgtggccgggaccctgagat |
| | | | ggggggaaagccgagaagga |
| | | | agaaccctcaggaaggcctg |
| | | | tacaatgaactgcagaaaga |
| | | | taagatggcggaggcctaca |
| | | | gtgagattgggatgaaaggc |
| | | | gagcgccggaggggcaaggg |
| | | | gcacgatggcctttaccagg |
| | | | gtctcagtacagccaccaag |
| | | | gacacctacgacgcccttca |
| | | | catgcaggccctgccccctc |
| | | | gcaccggtggccacgttgtt |
| | | | gaaggactggctggggaact |
| | | | tgaacaacttcgtgcacgac |
| | | | tggagcatcacccacaaggt |
| | | | caacgtgaaccaTGA |
| anti-HER2 | Nt | 37 | atggccttaccagtgaccgc |
| hsdAb1 | | | cttgctcctgccgctggcct |
| 41BB-z | | | tgctgctccacgccgccagg |
| (alfa tag) | | | cattctagagcggaagtgca |
| | | | gctgcaggcttccgggggag |
| | | | gatttgtgcagccggggggg |
| | | | tcattgcgactgagctgcgc |
| | | | cgcatccggagcaacatcaa |
| | | | acatcagtaacatgggctgg |
| | | | tttcgtcaggcccctggcaa |
| | | | ggagagagagttcgtttccg |
| | | | ccatctcccgtgcagaatcg |
| | | | cgtcctctgtattacgctga |
| | | | cagcgtaaagggaagattta |
| | | | caattagccgggataactcc |
| | | | aaaaacacggtctatctcca |
| | | | gatgaacagcctcagggccg |
| | | | aggacacagctacgtaTtac |
| | | | tgtgcatatatgcctctggt |
| | | | tcgtcacaaggcatactggg |
| | | | gacaggggacgcaggtaact |
| | | | gtgagtagccctgcaggaCC |
| | | | CAGCAGACTGGAGGAGGAGC |
| | | | TGAGAAGAAGACTGACCGAG |
| | | | CCCcatatgaccacgacgcc |
| | | | agcgccgcgaccaccaacac |
| | | | cggcgcccaccatcgcgtcg |
| | | | cagcccctgtccctgcgccc |
| | | | agaggcgtgccggccagcgg |
| | | | cggggggcgcagtgcacacg |
| | | | agggggctggacttcgcctg |
| | | | tgatatctacatctgggcgc |
| | | | ccttggccgggacttgtggg |
| | | | gtccttctcctgtcactggt |
| | | | tatcaccctttactgcaaac |
| | | | ggggcagaaagaaactcctg |
| | | | tatatattcaaacaaccatt |
| | | | tatgagaccagtacaaacta |
| | | | ctcaagaggaagatggctgt |
| | | | agctgccgatttccagaaga |
| | | | agaagaaggaggatgtgaac |
| | | | tgagagtgaagttcagcagg |
| | | | agcgcagacgcccccgcgta |
| | | | caagcagggccagaaccagc |
| | | | tctataacgagctcaatcta |
| | | | ggacgaagagaggagtacga |
| | | | tgttttggacaagagacgtg |
| | | | gccgggaccctgagatgggg |
| | | | ggaaagccgagaaggaagaa |
| | | | ccctcaggaaggcctgtaca |
| | | | atgaactgcagaaagataag |
| | | | atggcggaggcctacagtga |
| | | | gattgggatgaaaggcgagc |
| | | | gccggaggggcaaggggcac |
| | | | gatggcctttaccagggtct |
| | | | cagtacagccaccaaggaca |
| | | | cctacgacgcccttcacatg |
| | | | caggccctgccccctcgcac |
| | | | cggtggccacgttgttgaag |
| | | | gactggctggggaacttgaa |
| | | | caacttcgtgcacgactgga |
| | | | gcatcacccacaaggtcaac |
| | | | gtgaaccaTGA |
| anti HER2 | nt | 38 | atggccttaccagtgaccgc |
| sdAb1 | | | cttgctcctgccgctggcct |
| DAP10z | | | tgctgctccacgccgccagg |
| | | | cattctagagcggaagtgca |
| | | | gctgcaggcttccgggggag |
| | | | gatttgtgcagccggggggg |
| | | | tcattgcgactgagctgcgc |
| | | | cgcatccggagcaacatcaa |
| | | | acatcagtaacatgggctgg |
| | | | tttcgtcaggcccctggcaa |
| | | | ggagagagagttcgtttccg |
| | | | ccatctcccgtgcagaatcg |
| | | | cgtcctctgtattacgctga |
| | | | cagcgtaaagggaagattta |
| | | | caattagccgggataactcc |
| | | | aaaaacacggtctatctcca |
| | | | gatgaacagcctcagggccg |
| | | | aggacacagctacgtattac |
| | | | tgtgcatatatgcctctggt |
| | | | tcgtcacaaggcatactggg |
| | | | gacaggggacgcaggtaact |
| | | | gtgagtagccctgcaggaga |
| | | | gcagaagctgatctcagagg |
| | | | aggacctgggccggccaCAG |
| | | | ACGACTCCAGGAGAGAGATC |
| | | | ATCACTCCCTGCCTTTTACC |
| | | | CTGGCACTTCAGGCTCTTGT |
| | | | TCCGGATGTGGGTCCCTCTC |
| | | | TCTGCCGCTCCTGGCAGGCC |
| | | | TCGTGGCTGCTGATGCGGTG |
| | | | GCATCGCTGCTCATCGTGGG |
| | | | GGCGGTGTTCCTGTGCGCAC |
| | | | GCCCACGCCGCAGCCCCGCC |
| | | | CAAGAAGATGGCAAAGTCTA |
| | | | CATCAACATGCCAGGCAGGG |
| | | | GCcttaagAGAGTGAAGTTC |
| | | | AGCAGGAGCGCAGACGCCCC |
| | | | CGCGTACCAGCAGGGCCAGA |
| | | | ACCAGCTCTATAACGAGCTC |
| | | | AATCTAGGACGAAGAGAGGA |
| | | | GTACGATGTTTTGGACAAGA |
| | | | GACGTGGCCGGGACCCTGAG |
| | | | ATGGGGGGAAAGCCGCAGAG |
| | | | AAGGAAGAACCCTCAGGAAG |
| | | | GCCTGTACAATGAACTGCAG |
| | | | AAAGATAAGATGGCGGAGGC |
| | | | CTACAGTGAGATTGGGATGA |
| | | | AAGGCGAGCGCCGGAGGGGC |
| | | | AAGGGGCACGATGGCCTTTA |
| | | | CCAGGGTCTCAGTACAGCCA |
| | | | CCAAGGACACCTACGACGCC |
| | | | CTTCACATGCAGGCCCTGCC |
| | | | CCCTCGCaccggtggcCACG |
| | | | TTGTTGAAGGACTGGCTGGG |
| | | | GAACTTGAACAACTTCGTGC |
| | | | ACGACTGGAGCATCACccac |
| | | | aaggtcaacgtgaaccaTGA |
| HER2 | nt | 39 | atggccttaccagtgaccgc |
| sdAb1 | | | cttgctcctgccgctggcct |
| DAP12 CAR | | | tgctgctccacgccgccagg |
| | | | cattctagagcggaagtgca |
| | | | gctgcaggcttccgggggag |
| | | | gatttgtgcagccggggggg |
| | | | tcattgcgactgagctgcgc |
| | | | cgcatccggagcaacatcaa |
| | | | acatcagtaacatgggctgg |
| | | | tttcgtcaggcccctggcaa |
| | | | ggagagagagttcgtttccg |
| | | | ccatctcccgtgcagaatcg |
| | | | cgtcctctgtattacgctga |
| | | | cagcgtaaagggaagattta |
| | | | caattagccgggataactcc |
| | | | aaaaacacggtctatctcca |
| | | | gatgaacagcctcagggccg |
| | | | aggacacagctacgtattac |
| | | | tgtgcatatatgcctctggt |
| | | | tcgtcacaaggcatactggg |
| | | | gacaggggacgcaggtaact |
| | | | gtgagtagccctgcaggaga |
| | | | gcagaagctgatctcagagg |
| | | | aggacctgggccggccaCTC |
| | | | CGTCCTGTCCAGGCCCAGGC |
| | | | CCAGAGCGATTGCAGTTGCT |
| | | | CTACGGTGAGCCCGGGCGTG |
| | | | CTGGCAGGGATCGTGATGGG |
| | | | AGACCTGGTGCTGACAGTGC |
| | | | TCATTGCCCTGGCCGTGTAC |
| | | | TTCCTGGGCCGGCTGGTCCC |
| | | | TCGGGGGCGAGGGGCTGCGG |
| | | | AGGCAGCGACCCGGAAACAG |
| | | | CGTATCACTGAGACCGAGTC |
| | | | GCCTTATCAGGAGCTCCAGG |
| | | | GTCAGAGGTCGGATGTCTAC |
| | | | AGCGACCTCAACACACAGAG |
| | | | GCCGTATTACAAACACGTTG |
| | | | TTGAAGGACTGGCTGGGGAA |
| | | | CTTGAACAACTTCGTGCACG |
| | | | ACTGGAGCATCACccacaag |
| | | | gtcaacgtgaaccaTGA |
| HER2 sdAb | nt | 40 | atggccttaccagtgaccgc |
| nº2 41-z | | | cttgctcctgccgctggcct |
| CAR | | | tgctgctccacgccgccagg |
| | | | cattctagaGCGGAAGTGCA |
| | | | GCTGCAGGCTTCCGGGGGAG |
| | | | GATTTGTGCAGCCGGGGGGG |
| | | | TCATTGCGACTGAGCTGCGC |
| | | | CGCATCCGGAGATTCCTACA |
| | | | ACGAGAGTTCTATGGGCTGG |
| | | | TTTCGTCAGGCCCCTGGCAA |
| | | | GGAGAGAGAGTTCGTTTCCG |
| | | | CCATCTCGGCACGTGGTAAC |
| | | | CATCCTCTGTATTACGCTGA |
| | | | CAGCGTAAAGGGAAGATTTA |
| | | | CAATTAGCCGGGATAACTCC |
| | | | AAAAACACGGTCTATCTCCA |
| | | | GATGAACAGCCTCAGGGCCG |
| | | | AGGACACAGCTACGTATTAC |
| | | | TGTGCATCGATGCCTATGCC |
| | | | TAAGTGGAAGAAGTACTGGG |
| | | | GACAGGGGACGCAGGTAACT |
| | | | GTGAGTAGCcctgcaggaga |
| | | | gcagaagctgatctcagagg |
| | | | aggacctgcatatgaccacg |
| | | | acgccagcgccgcgaccacc |
| | | | aacaccggcgcccaccatcg |
| | | | cgtcgcagcccctgtccctg |
| | | | cgcccagaggcgtgccggcc |
| | | | agcggggggggcgcagtgca |
| | | | cacgagggggctggacttcg |
| | | | cctgtgatatctacatctgg |
| | | | gcgcccttggccgggacttg |
| | | | tggggtccttctcctgtcac |
| | | | tggttatcaccctttactgc |
| | | | aaacggggcagaaagaaact |
| | | | cctgtatatattcaaacaac |
| | | | catttatgagaccagtacaa |
| | | | actactcaagaggaagatgg |
| | | | ctgtagctgccgatttccag |
| | | | aagaagaagaaggaggatgt |
| | | | gaactgagagtgaagttcag |
| | | | caggagcgcagacgcccccg |
| | | | cgtacaagcagggccagaac |
| | | | cagctctataacgagctcaa |
| | | | tctaggacgaagagaggagt |
| | | | acgatgttttggacaagaga |
| | | | cgtggccgggaccctgagat |
| | | | ggggggaaagccgagaagga |
| | | | agaaccctcaggaaggcctg |
| | | | tacaatgaactgcagaaaga |
| | | | taagatggcggaggcctaca |
| | | | gtgagattgggatgaaaggc |
| | | | gagcgccggaggggcaaggg |
| | | | gcacgatggcctttaccagg |
| | | | gtctcagtacagccaccaag |
| | | | gacacctacgacgcccttca |
| | | | catgcaggccctgccccctc |
| | | | gcaccggtggccacgttgtt |
| | | | gaaggactggctggggaact |
| | | | tgaacaacttcgtgcacgac |
| | | | tggagcatcacccacaaggt |
| | | | caacgtgaaccaTGA |
| scFv CD19 | nt | 41 | ATGGCCTTACCAGTGACCGc |
| 41-z CAR | | | cttgctcctgccgctggcct |
| | | | tgctgctccacgccgccagg |
| | | | ccggatatccagatgacaca |
| | | | gactacatcctccctgtctg |
| | | | cctctctgggagacagagtc |
| | | | accatcagttgcagggcaag |
| | | | tcaggacattagtaaatatt |
| | | | taaattggtatcagcagaaa |
| | | | ccagatggaactgttaaact |
| | | | cctgatctaccatacatcaa |
| | | | gattacactcaggagtccca |
| | | | tcaaggttcagtggcagtgg |
| | | | gtctggaacagattattctc |
| | | | tcaccattagcaacctggag |
| | | | caagaagatattgccactta |
| | | | cttttgccaacagggtaata |
| | | | cgcttccgtacacgttcgga |
| | | | ggggggaccaagctggagat |
| | | | cacaggtggcggtggctcgg |
| | | | gcggtggtgggtcgggtggc |
| | | | ggcggatctgaggtgaaact |
| | | | gcaggagtcaggacctggcc |
| | | | tggtggcgccctcacagagc |
| | | | ctgtccgtcacatgcactgt |
| | | | ctcaggggtctcattacccg |
| | | | actatggtgtaagctggatt |
| | | | cgccagcctccacgaaaggg |
| | | | tctggagtggctgggagtaa |
| | | | tatggggtagtgaaaccaca |
| | | | tactataattcagctctcaa |
| | | | atccagactgaccatcatca |
| | | | aggacaactccaagagccaa |
| | | | gttttcttaaaaatgaacag |
| | | | tctgcaaactgatgacacag |
| | | | ccatttactactgtgccaaa |
| | | | cattattactacggtggtag |
| | | | ctatgctatggactactggg |
| | | | gccaaggaacctcagtcacc |
| | | | gtctcctcacctgcaggaga |
| | | | gcagaagctgatctcagagg |
| | | | aggacctgcatatgaccacg |
| | | | acgccagcgccgcgaccacc |
| | | | aacaccggcgcccaccatcg |
| | | | cgtcgcagcccctgtccctg |
| | | | cgcccagaggcgtgccggcc |
| | | | agcggggggggcgcagtgca |
| | | | cacgagggggctggacttcg |
| | | | cctgtgatatctacatctgg |
| | | | gcgcccttggccgggacttg |
| | | | tggggtccttctcctgtcac |
| | | | tggttatcaccctttactgc |
| | | | aaacggggcagaaagaaact |
| | | | cctgtatatattcaaacaac |
| | | | catttatgagaccagtacaa |
| | | | actactcaagaggaagatgg |
| | | | ctgtagctgccgatttccag |
| | | | aagaagaagaaggaggatgt |
| | | | gaactgagagtgaagttcag |
| | | | caggagcgcagacgcccccg |
| | | | cgtacaagcagggccagaac |
| | | | cagctctataacgagctcaa |
| | | | tctaggacgaagagaggagt |
| | | | acgatgttttggacaagaga |
| | | | cgtggccgggaccctgagat |
| | | | ggggggaaagccgagaagga |
| | | | agaaccctcaggaaggcctg |
| | | | tacaatgaactgcagaaaga |
| | | | taagatggcggaggcctaca |
| | | | gtgagattgggatgaaaggc |
| | | | gagcgccggaggggcaaggg |
| | | | gcacgatggcctttaccagg |
| | | | gtctcagtacagccaccaag |
| | | | gacacctacgacgcccttca |
| | | | catgcaggccctgccccctc |
| | | | gcaccggtggccacgttgtt |
| | | | gaaggactggctggggaact |
| | | | tgaacaacttcgtgcacgac |
| | | | tggagcatcacccacaaggt |
| | | | caacgtgaaccaTGA |

### FIGURES LEGENDS

FIG. 1: Subtractive selection led to conformational or cell type specific hs2dAbs. (A) Tumor cell surface subtractive selection scheme. (B) Determination of the specificity of anti HER2 sdAbs (1-5) by ELISA on HER2 fused with a rabbit Fc (HER2 rFc) or on a control rabbit IgG (rIgG) (C) anti-HER2 hsdAbs 1, 2 and 5 decorated the SKBR3 membrane in immunofluorescence: SKBR3 cells were fixed with 1% paraformaldehyde and stained with hsdAb1, 2 or 5 revealed by an anti-HisTag (Sigma) and an anti-MouseCy3 secondary antibody (Jackson). (D) FACS analysis of anti-HER2 hsdAbs no 1, 2 and 5 on SKBR3 HER2 positive cells versus MCF10A HER2 negative cells..

FIG. 2: Immunofluorescence on xenografted murine model Immunofluorescence with injected anti-HER2 hsdAb no 1 coupled with a human Fc domain or with trastuzumab (positive control). Tumors were recovered after 96 hours, sectioned along the median axis (vibratome cuts 50 μm BC911 xenograft (HER2+)), and labelled with secondary anti-Human Fc Cy3.

FIG. 3: Cytotoxicity of T cells expressing anti-HER2 hsdAb 41BB-z (HER2-41-z) CARs against a HER2 positive SKBR3 cancer cells line evaluated by crystal violet using two independent CD8+ T cell donors.

The CARs used for the CD8 T donor cell transduction were composed of an anti-HER2 sdAb (HER2-CAR) as herein described or of a scFv directed against a CD19 antigen (CD19 scFv-CAR) fused in its N terminal domain to a CD8 transmembrane domain followed by 4-1BB and CD3zeta intracellular stimulatory domains and with a SBP (streptavidin-binding peptide) tag in the C-terminus, referred herein as CD19-41-z. These data are representative of more than four independent CD8+ T cell donors.

FIG. 4: Cytotoxicity of HER2 hsdAb CARs with different activation domains against HER2 positive SKBR3 breast cancer cells line.

4A. Cytotoxicity evaluation by a xCELLigence assay against SKBR3 breast cancer cell line, using T cells expressing CAR T composed by the anti HER2 sdAb No 1 fused to different stimulatory domains and with a SBP in the C-terminus.

A first-generation CAR DAP12 including the full stimulatory protein DAP12 (HER2 sdAb1-DAP12) and a second generation CAR, including the full DAP10 protein and the CD3z intracellular domain, also named herein DAP10z (named herein HER2 sdAb1-DAP10-CD3, or HER2 sdAb1-DAP10-z), which therefore comprises an additional CD3zeta domain fused in the N-terminal to DAP10 as compared to the first generation of CAR, were both compared to a classical CAR design, wherein the HER2 sdAb is fused in its C terminal domain with the transmembrane domain of CD8 followed by the 4-1BB, CD3zeta intracellular domains and a SBP tag (this CAR is also named HER2 sdAb-41-z). The arrows indicate the time of CAR T addition. This assay was reproduced with two independent donors.

4B. Crystal Violet cytotoxicity assay determined by the killing of luminescent target cells, SKBR3 as positive HER2 cells and RPE-1 as low or negative HER2 cells. CAR T cells were generated from two independent donors (B and C) and confronted with the target cells. Around 72h later, the luminescence was determined with a low level associated to a higher killing. The luminescence values were normalized for highest survival and converted to percentage of cell death. Highest tumor killing was observed T cells expressing the HER2 sdAb-41-z, the HER2 sdAb DAP10z and the CD19 scFv-41BBz (scFv-41-z), which was also associated with higher killing for the non-tumor cell line (RPE-1). T cells expressing the HER2 sdAb DAP12 CAR exhibit low cytotoxic activity at lower effector to target ratio but similar to the other CARs at higher effector to target ratio, with the lowest activity against normal cell line. Similar results were observed for donors B and C.

FIG. 5: Cytotoxicity of different clones of HER2 hsdAb CARs (hsdAbs no 1, and 2) against HER2 positive SKBR3 breast cancer cells line. Real-time cell death analysis using an xCELLigence assay was used to evaluate the cytotoxicity of the different HER2 hsdAb CARs (shAb1 and 2) against the SKBR3 cell line. The CD8 T cells used in this assay were isolated from two different donors A and B.

FIG. 6: A-Transduction efficacy and survival of Primary T cells. B-Adoptively transferred CAR T cells efficiently controlled tumor growth in tumor bearing mice recipients.

### EXAMPLES

### 1. Material and Methods

### In Vitro Experiments

Affinity measurements: Affinity measurements can be done by surface plasmon resonance (for example as detailed in Moutel, Sandrine et al. “NaLi-H1: A universal synthetic library of humanized nanobodies providing highly functional antibodies and intrabodies.” eLife vol. 5 e16228. 19 Jul. 2016, doi:10.7554/eLife.16228). More particularly, binding affinities of selected hs2dAb fused to a 10HIS tag measured by surface plasmon resonance single cycle kinetics method. Dissociation equilibrium constant K_{D} corresponds to the ratio between off-rate and on-rate kinetic constant K_{off}/Kₒₙ. Non relevant hs2dAb were used as negative controls and gave no detectable binding signal. Affinity measurements were also performed on an Octet-HTX (Sartorius) by bio-Layer Interferometry (BLI) which is an optical technique for measuring macromolecular interactions by analyzing interference patterns of white light reflected from the surface of a biosensor tip. BLI experiments were used to determine the kinetics and affinity of molecular interactions. Biosensors with proteinA were used to capture recombinant human Her2/ErbB2 Fc Tag (ACROBiosystems), the sensors were then dipped in wells containing purified sdAb to measure kinetics at 37° C.

Flow cytometry: For HER2 immunoassay, cell surface staining can be performed in phosphate-buffered saline (PBS) supplemented with 1% SFV. 100 μL of supernatant (80 μL phages+20 μL PBS/milk1%) can be incubated on 1·105 cells for 1 hr on ice. Phage binding can be detected by a 1:250 dilution of anti-M13 antibody (GE healthcare, France) for 1 hr on ice followed by a 1:400 dilution of Cy5-conjugated anti-Mouse antibody (Jackson ImmunoResearch, Europe Ltd) for 45 min. Samples can be analyzed by flow cytometry on a FACSCalibur using CellQuest Pro software (BD Biosciences, France).

### In Vivo Experiments

### Transduction of T Cells

Production of lentiviral particles containing the CAR HER2-41-z construct using the packaging plasmid psPAX2 (12260; Addgene) and envelop plasmid pVSVG (pMD2.G; 12259, Addgene) was done in HEK293FT cells.

Primary CD4/CD8+ T cells isolated from PBMCs were plated into culture plates and transduced with HER2 CAR lentiviral particle at a MOI between 1 and 5 for 3 days in TexMacs buffer supplemented with 10 ng/mL IL7/IL15. Transduced primary CD4/CD8+ T cells were analyzed 6-7 days post-transduction by Flow cytometry assay to evaluate cell killing and transduction efficiency.

### Flow Cytometry

Transduced cells were centrifuged (300 g, 4° C., 5 minutes), washed twice in cold 1×PBS (300 g, 4° C., 5 minutes) and incubated with live/dead fixable staining (20 minutes, on ice; Thermofisher). The cells were then washed twice in cold PBS (300 g, 4° C., 5 minutes) or FACS buffer (1×PBS, 1% BSA, 0.05% sodium azide, 1 mL EDTA 0.5 M, filtered and kept at 4° C.) and, when not analyzed immediately, the cells were fixed in 3% PFA-1×PBS (10 minutes, RT) and washed twice in 1×PBS.

### Animal Experiments

NGS mice were housed in SPF conditions in the animal facilities in Institute Curie. Live animal experiments were performed in accordance to the national guidelines. One million of SK-OV-3/Luc ovarian carcinoma cell line (CellBiolabs) in PBS were intravenously (i.v.) injected in immunodeficient NSG mouse (NOD scid gamma mouse) at day 0. After 21 days, 1,4 millions of CAR HER2-41-z T cells in PBS were i.v. injected in immunodeficient NSG mouse (NOD scid gamma mouse).

Bioluminescence imaging of the mice was performed in IVIS Optical Imaging (Perkin Elmer). Mice was injected with 150 mg/kg D-luciferin, anesthetized by isoflurane inhalation and imaged after 10⁻¹⁵ min (peak of emission). Signal quantification in specific regions of interest (ROIs) was determined.

### 2. Results

Targeting tumor-specific epitope is essential for various diagnostics and therapeutic approaches. Single domain antibodies or Nanobodies®, notably Camelid natural single domain VH referred to as VHH, can be expressed as recombinant fragments They represent attractive alternatives over classical antibody fragments like scFvs because they are easy to manipulate and they are not limited by potential misfolding of the two domains (Worn and Pluckthun, 1999). It is noteworthy that VHH FRWs show a high sequence and structural homology with human VH domains of family III (Muyldermans, 2013) and VHH have comparable immunogenicity as human VH (Bartunek et al., 2013; Holz et al., 2013). Thus, they further constitute very interesting agents for therapeutic applications.

### Single Domain Antibody Identification

The inventors previously disclosed a synthetic single domain antibody library. Unique features in framework regions of single domain antibodies were identified thus allowing to obtain a highly stable single domain antibody scaffold and its use in generating synthetic single domain antibody library, such as synthetic single domain antibody phage display library (see WO2015063331 and Moutel et al., eLife 2016; 5:e16228).

Said synthetic single domain antibody library was now screened against HER2.

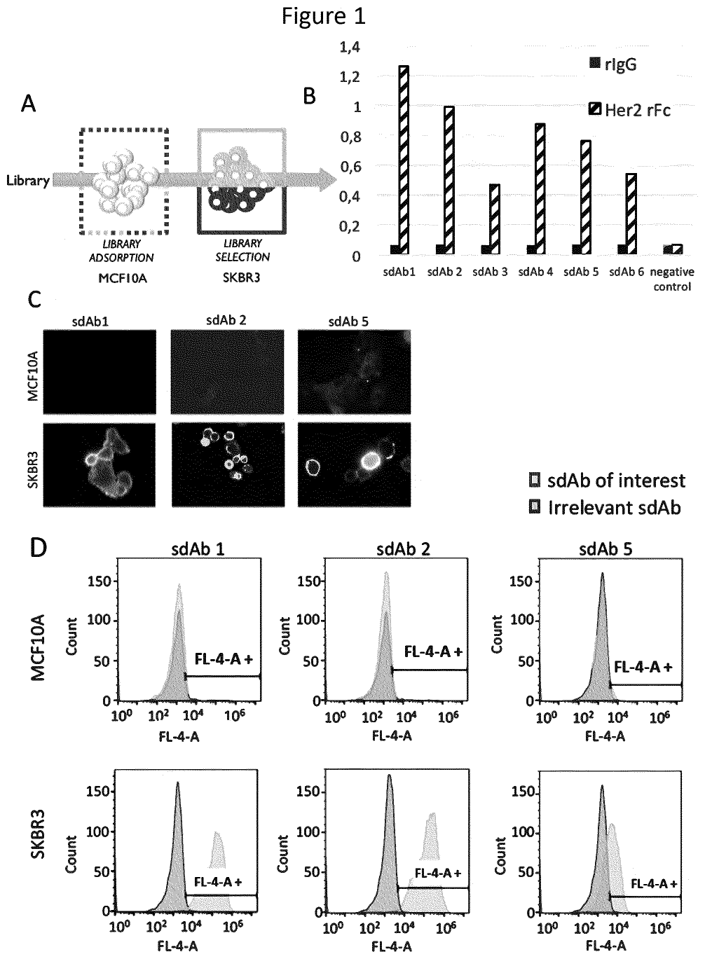

A subtractive selection scheme was set up to identify antibodies selectively detecting the surface of breast tumor cells: phages displaying hs2dAbs were first depleted against a reference cell line before being selected against the target one (FIG. 1A). As a target cell line, the SKBR3 line was used, because it overexpresses the HER2 cell surface protein, while the MCF10A cell line, negative for HER2, was used to pre-adsorb the library. After the third round of bio-panning, clones were analyzed by FACS and tested on SKBR3 cells and on MCF10A cells. Sequencing the clones tested positive on SKBR3 revealed that binders can been selected.

Six single domain antibodies (sdAbs) directed against the breast tumor antigen HER2 have now been identified as above mentioned and developed for therapeutic applications, notably cancer therapeutic applications. Detection of HER2 at the cell surface can be achieved by immunofluorescence or by FACS (see FIGS. 1C and 1D).

| sdAb | | | | | | | |
|---|---|---|---|---|---|---|---|
| (Nº) | FR1 | CDR1 | FR2 | CDR2 | FR3 | CDR3 | FR4 |
| 1 | MAEVQLQA | AT | MGWFRQ | RA | YYADSVKGRFTISRD | YMP | YWGQ |
| | SGGGFVQP | SN | APGKERE | ES | NSKNTVYLQMNSLR | LVR | GTQV |
| | GGSLRLSC | IS | FVSAIS | RP | AEDTATYYCA | HKA | TVSS |
| | AASG | N | | L | | | |
| 2 | MAEVQLQA | DS | MGWFRQ | AR | YYADSVKGRFTISRD | SMP | YWGQ |
| | SGGGFVQP | YN | APGKERE | GN | NSKNTVYLQMNSLR | MPK | GTQV |
| | GGSLRLSC | ES | FVSAIS | HP | AEDTATYYCA | WK | TVSS |
| | AASG | S | | L | | K | |
| 3 | MAEVQLQA | RY | MGWFRQ | EY | YYADSVKGRFTISRD | IRH | YWGQ |
| | SGGGFVQP | YE | APGKERE | GG | NSKNTVYLQMNSLR | QNQ | GTQV |
| | GGSLRLSC | QS | FVSAIS | W | AEDTATYYCA | SMM | TVSS |
| | AASG | I | | QH | | | |
| 4 | MAEVQLQA | YT | MGWFRQ | RD | YYADSVKGRFTISRD | MIP | YWGQ |
| | SGGGFVQP | SR | APGKERE | QD | NSKNTVYLQMNSLR | LTR | GTQV |
| | GGSLRLSC | ES | FVSAIS | GI | AEDTATYYCA | NAK | TVSS |
| | AASG | G | | S | | | |
| 5 | MAEVQLQA | YT | MGWFRQ | W | YYADSVKGRFTISRD | VTP | YWGQ |
| | SGGGFVQP | FS | APGKERE | NH | NSKNTVYLQMNSLR | LPP | GTQV |
| | GGSLRLSC | EE | FVSAIS | TF | AEDTATYYCA | NKA | TVSS |
| | AASG | T | | FE | | | |
| 6 | MAEVQLQA | RG | MGWFRQ | WT | YYADSVKGRFTISRD | MM | YWGQ |
| | SGGGFVQP | YT | APGKERE | SN | NSKNTVYLQMNSLR | PLP | GTQV |
| | GGSLRLSC | GT | FVSAIS | QT | AEDTATYYCA | RW | TVSS |
| | AASG | T | | S | | KG | |

Humanized anti (ER2 sdAbs of the present invention have a K_{D} (see above) comprised between 1 and 100·10⁹, notably between 1 and 0·10⁹, between 1 and 5·10⁻⁹, or between 5 and 100·10⁻⁹ notably between 10 and 100·10⁻⁹, more particularly between 50·10⁻¹⁰ and 100·10⁻⁹. The 6 synthetic single domain antibodies (s2dAbs) that have now been identified are highly stable and have low risks of immunogenicity. They further exhibit an affinity of binding for HTER2 between 10⁻⁸ and 1·10⁻¹¹ M.

Affinity measurement were also performed using BLI as above detailed. Similar affinity values were obtained.

| sdAb | K_{D} (nM) | Ka (M⁻¹s⁻¹) | Kdis (s⁻¹) |
|---|---|---|---|
| 1 | 4.6 | 8.8 × 10⁵ | 4.0 × 10⁻³ |
| 2 | 4.1 | 12 × 10⁵ | 4.9 × 10⁻³ |
| 4 | 5.2 | 14.7 × 10⁵ | 7.6 × 10⁻³ |
| 5 | 33.3 | 0.44 × 10⁵ | 1.5 × 10⁻³ |
| 6 | 8.4 | 7.8 × 10⁵ | 6.6 × 10⁻³ |

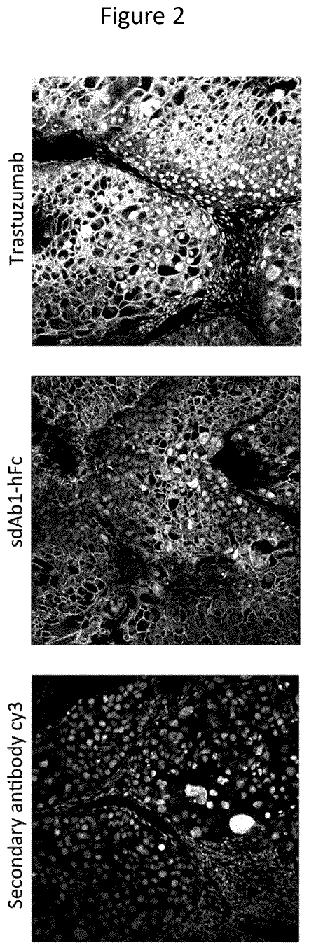

The capacity of the IgG-like reconstituted antibodies produced in CHO supernatant cells to target a HER2-positive tumor in vivo can be investigated using a xenografted murine model. Animals were injected either with anti-HER2 sdAb no 1 coupled with an Fc fragment or with trastuzumab (positive control) Tumors were recovered after 96 hours, sectioned along the median axis to have access to the inner section, and labelling with secondary anti-HumanFc was performed. The mass (80 kDa) of reconstituted IgG-like Abs impairs direct kidney filtration and the rapid clearance specific of monovalent sdAbs. Consequently, sdAb1-hFc accumulated in the tumor tissues with the same kinetic of the positive control trastuzumab. Although fluorescence does not allow for accurate quantitative comparisons, the recombinant antibodies seem as effective as trastuzumab in concentrating inside the tumor (see FIG. 2).

Anti HER2 sdAb no 1 has also been injected in mice in various forms (sdAb only, dimeric and fused to an Fc). The apparent pK was increased with the size and the antibody can be found in grafted tumors (data not shown).

### Design and Efficacy of Chimeric Antigen Receptors Comprising a Single Domain Antibody Directed Against HER2 as Herein Described

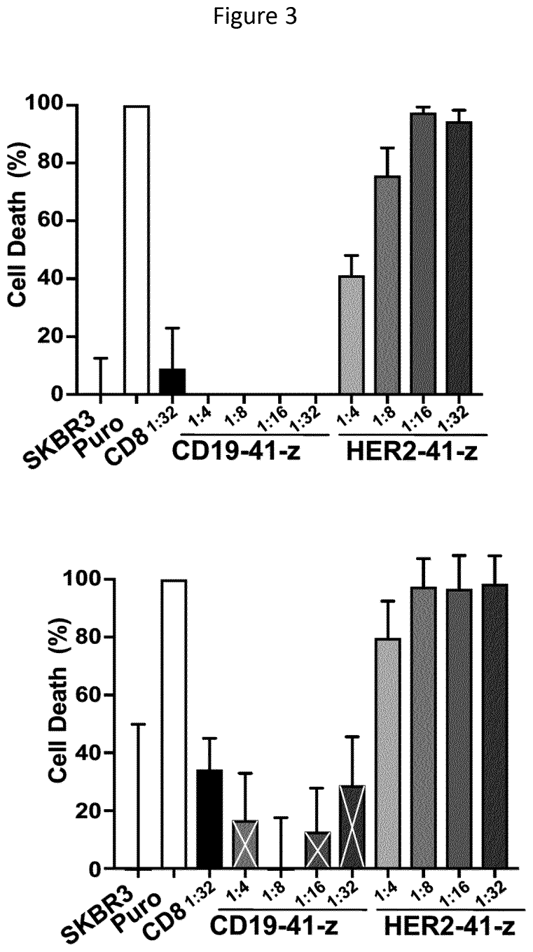

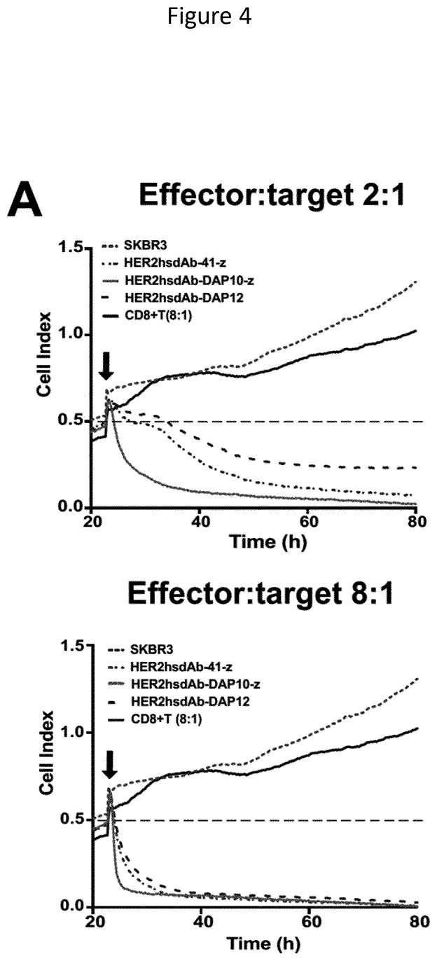

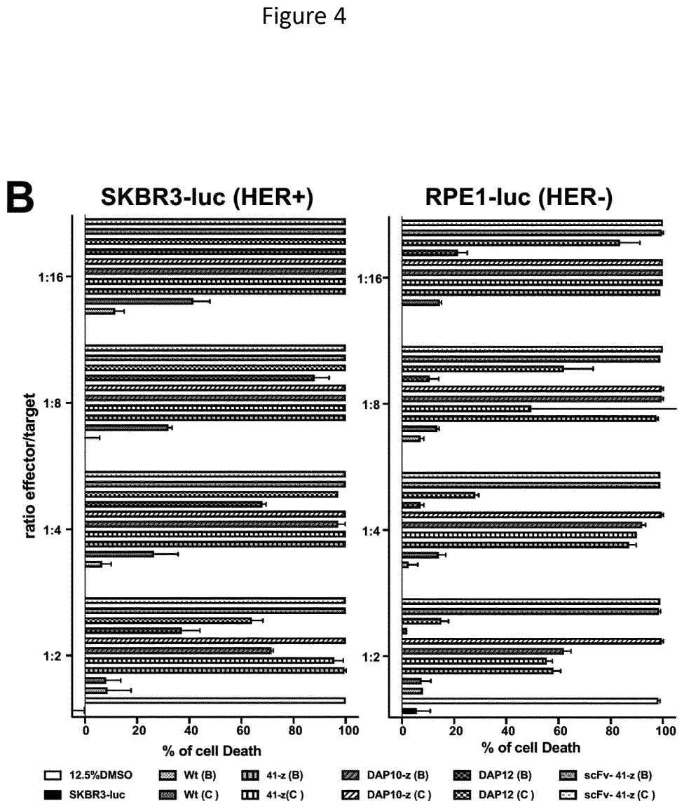

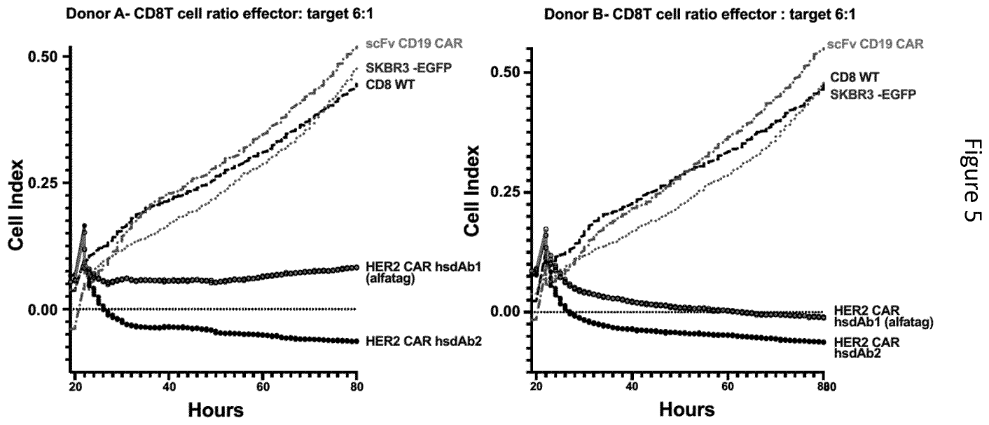

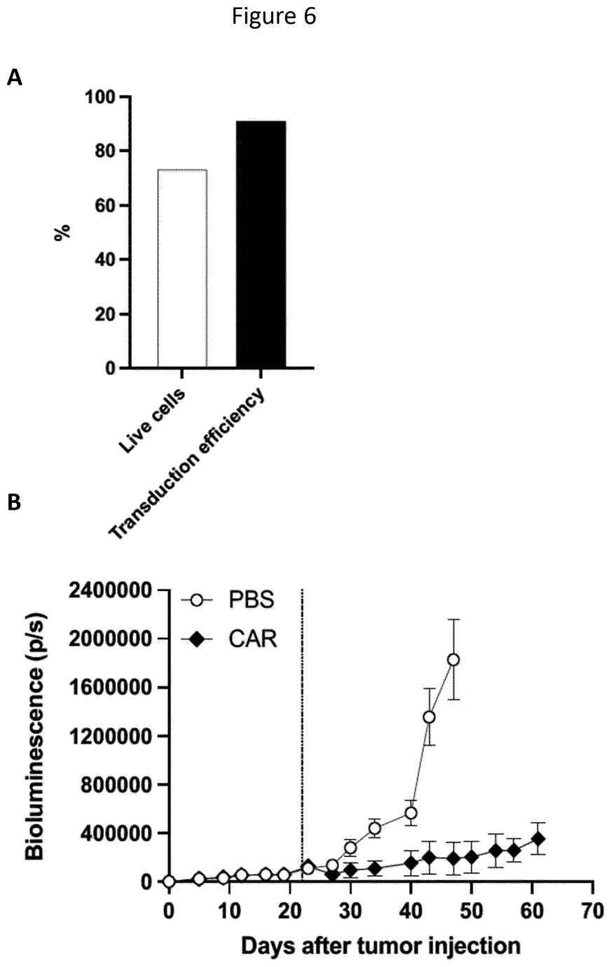

Chimeric Antigen Receptors with various designs, using the humanized anti-HER2 sdAbs as above defined, and aimed at targeting solid tumors expressing HER2 (see FIGS. 3 and 4) were developed.

The efficacy of the HER2 sdAbs as herein described (in particular of HER2 sdAb no 1) as a CAR was confirmed using various scaffolds: the current scaffold used in clinics (41BB-CD3zeta, also named herein 41-z, see Milone M C, Fish J D, Carpenito C, et al. Chimeric receptors containing CD137 signal transduction domains mediate enhanced survival of T cells and increased antileukemic efficacy in vivo [published correction appears in Mol Ther. 2015 July; 23(7):1278]. Mol Ther. 2009; 17(8):1453-1464) (FIGS. 3-5) and newly developed CAR scaffolds (DAP10-z and DAP12 based scaffolds, FIGS. 4-5).

Several results have been obtained demonstrating the applicability and activity of a CAR-based HER2 sdAb as herein described against solid tumor using in vitro assays, such as xCELLigence, crystal violet and luciferase target cell-based assays.

The hssdAb (for human synthetic single domain antibody, or hs2dAb) against the anti-HER2 hsdAb no 1 was fused in C-terminus to 41-z CARs comprising the signaling domains 4-1BB and CD3zeta (CD3z), followed by SBP tag (see the legend of FIGS. 3 and 4). The cytotoxicity of these CARs was validated using the crystal violet in vitro assay that allow to evaluate the percentage of target cell death upon incubation with the effector T cells expressing the CAR construct (see Figure. 3). These results provide evidence that anti-HER2 sdAb based CARs of the present invention and in particular the anti-HER2 sdAb no 1 41-z CAR was highly efficient in killing the tumor highly expressing HER2 (in the present example the tumor breast cell line SKBR3) at different target to effector ratio (FIG. 3).

It has been further tested whether the HER2 sdAbs as herein described could be used in other CARs with different activation domain(s) that were previously validated for scFv-CD19 CARs. These includes the DAP10-CD3zeta based CAR (HER2 hsdAb-DAP10-CD3, also referred as DAP10-z) and the DAP12 based CAR as a 1^{st} generation CAR (HER2 sdAb-DAP12) (FIG. 3). To evaluate the cytotoxicity of these CARs, two independent assays were used: the xCELLigence (FIG. 4A) and the measurements of luminescence levels of the target cell line, as a proxy of their viability (FIG. 4B).

Using the xCELLigence assay, it was observed that all the CARs can efficiently kill SKBR3 cancer cell line when transduced in effector T cells. However, using various effector-to-target cell ratio, it has been verified that the most cytotoxic CARs are HER2 sdAb-DAP10-z and HER2 sdAb 41-z based CARs, while the HER2 sdAb-DAP12 based CAR is slightly less efficient (FIG. 4A).

These results were reproduced using luciferase target cells activity (FIG. 4B). In this luciferase-based assay, the cytotoxicity of a scFv against HER2, (previously used as a CAR in clinical trials and as a therapeutic antibody (Traztuzumab) was also evaluated. The cytotoxicity against tumor cells of the HER2 scFv based CAR in comparison to the HER2 sdAb (as herein described) -based CAR was similar, however the HER2 scFv-based CAR was also very efficient in killing non-tumor cells lines (RPE-1) while the HER sdAb-based CAR was slightly lower cytotoxic (FIG. 4B). The lower cytotoxicity against non-tumor cell lines was even more prominent when using the HER2 sdAb CAR based on DAP12 (FIG. 4B). These data provide evidence that the lower efficiency associated to DAP12 should be advantageous as it may induce less CRS while enabling a more specific response.

HER2 hsdAbs no 1 and 2 were built in a 41-z CAR-based design as previously described. Killing efficiency of HER2 positive cells (e.g. SKBR3 cells) by effector T cells expressing these constructs were compared using real-time cell killing, the xCELLigence assay (FIG. 5). It was observed that effector T cells (obtained from 2 different donors, FIG. 5 left and right) expressing the HER2 sdAb no 1 and 2 41-z based CAR constructs were highly efficient in killing the target cells using T cells. Effector T cells expressing the scFv CD19 41-z CAR were used as control.

Finally, primary T cells were transduced with the CAR HER2-41-z construct as herein described (see also below for details) with an efficiency of about 90% and with a percentage of survival of around 70% (FIG. 6A). The CAR T cells were then injected i.v. at day 21 post-tumor cell i.v. administration (FIG. 6B). The tumor was efficiently controlled by CAR HER2-41-z while in the group without CAR T cells, the tumor augment significantly (FIG. 2), demonstrating the HER2 CAR efficiency of against tumor grow.

### Car Constructs:

anti-HER2 hsdAb1 41BB-z (myc tag)
> [nº1-n&vHHHER2 myc.xdna-1236 bp] Fragment
extracted from 3658pTRIP-SFFV-BFP-
2a-VHHaHer2ju.xdna [between 4504 and 5739].
atggccttaccagtgaccgccttgctcctgccgctggccttgctg
ctccacgccgccaggcattctagagcggaagtgcagctgcaggct
tccgggggaggatttgtgcagccgggggggtcattgcgactgagc
tgcgccgcatccggagcaacatcaaacatcagtaacatgggctgg
tttcgtcaggcccctggcaaggagagagagttcgtttccgccatc
tcccgtgcagaategcgtectctgtattacgctgacagcgtaaag
ggaagatttacaattagccgggataactccaaaaacacggtctat
ctccagatgaacagcctcagggccgaggacacagctacgtattac
tgtgcatatatgcctctggttcgcacaaggcatactggggacagg
ggacgcaggtaactgtgagtagccctgcaggagagcagaagctga
tctcagaggaggacctgcatatgaccacgacgccagcgccgcgac
caccaacaccggcgcccaccatcgcgtcgcagcccctgtccctgc
gcccagaggcgtgccggccagcggggggggcgcagtgcacacgag
ggggctggacttcgcctgtgatatctacatctgggcgcccttggc
cgggacttgtggggtccttctcctgtcactggttatcacccttta
ctgcaaacggggcagaaagaaactcctgtatatattcaaacaacc
atttatgagaccagtacaaactactcaagaggaagatggctgtag
ctgccgatttccagaagaagaagaaggaggatgtgaactgagagt
gaagttcagcaggagcgcagacgcccccgcgtacaagcagggcca
gaaccagctctataacgagctcaatctaggacgaagagaggagta
cgatgttttggacaagagacgtggccgggaccctgagatgggggg
aaagccgagaaggaagaaccctcaggaaggcctgtacaatgaact
gcagaaagataagatggcggaggcctacagtgagattgggatgaa
aggcgagcgccggaggggcaaggggcacgatggcctttaccaggg
tctcagtacagccaccaaggacacctacgacgcccttcacatgca
ggccctgccccctcgcaccggtggccacgttgttgaaggactggc
tggggaacttgaacaacttcgtgcacgactggagcatcacccaca
aggtcaacgtgaaccaTGA
Features:
Myc-Tag: [439:468]
insert from PCR(BamHI-spCD8-XbaI-
VHHaHer2-SbfI oligos gBlock): [805:1140]
SP CD8: [1:63]
sdAb1: [70:429]
Hinge CD8: [475:609]
TM CD8: [610:681]
CD3zeta: [808:1143]
Small SBP: [1147:1233]
anti-HER2 hsdAb1 41BB-z (alfa tag)
> [n°1-n&vHHHER2 alfa.xdna-1251 bp] Fragment
extracted from pTRIP-SFFV-BFP-2a-
CARVHHaHer.xdna [between 4504 and 5754].
atggccttaccagtgaccgccttgctcctgccgctggccttgctg
ctccacgccgccaggcattctagageggaagtgcagctgcaggct
tccgggggaggatttgtgcagccgggggggtcattgcgactgagc
tgcgccgcatccggagcaacatcaaacatcagtaacatgggctgg
tttcgtcaggcccctggcaaggagagagagttcgtttccgccatc
tcccgtgcagaategcgtectctgtattacgctgacagcgtaaag
ggaagatttacaattagccgggataactccaaaaacacggtctat
ctccagatgaacagcctcagggccgaggacacagctacgtaTtac
tgtgcatatatgcctctggttcgtcacaaggcatactggggacag
gggacgcaggtaactgtgagtagccctgcaggaCCCAGCAGACTG
GAGGAGGAGCTGAGAAGAAGACTGACCGAGCCCcatatgaccacg
acgccagegccgegaccaccaacaceggegcccaccatcgcgtcg
cagcccctgtccctgcgcccagaggcgtgccggccagcggcgggg
ggcgcagtgcacacgagggggctggacttcgcctgtgatatctac
atctgggcgcccttggccgggacttgtggggtccttctcctgtca
ctggttatcaccctttactgcaaacggggcagaaagaaactcctg
tatatattcaaacaaccatttatgagaccagtacaaactactcaa
gaggaagatggctgtagctgccgatttccagaagaagaagaagga
ggatgtgaactgagagtgaagttcagcaggagcgcagacgccccc
gcgtacaagcagggccagaaccagctctataacgagctcaatcta
ggacgaagagaggagtacgatgttttggacaagagacgtggccgg
gaccctgagatggggggaaagccgagaaggaagaaccctcaggaa
ggcctgtacaatgaactgcagaaagataagatggcggaggcctac
agtgagattgggatgaaaggcgagcgccggaggggcaaggggcac
gatggcctttaccagggtctcagtacagccaccaaggacacctac
gacgcccttcacatgcaggccctgccccctcgcaccggtggccac
gttgttgaaggactggctggggaacttgaacaacttcgtgcacga
ctggagcatcacccacaaggtcaacgtgaaccaTGA
Features:
SP CD8: [1:63]
vHH: [70:429]
Alphatag internal: [439:483]
Hinge CD8: [490:624]
TM CD8: [625:696]
AD 4-1BB: [697:822]
CD3zeta[mod]: [823:1158]
SBPdel−: [1162:1248]
anti HER2 sdAb1 DAP10z
> [sdAb nº1-DAP10CD3-SBP.xdna-1140 bp]
Fragment extracted from pTRIP-SFFV-BFP-2a-
vhhHER2-Myc-DAP10CD3-SBP
[between 4504 and 5643]
atggccttaccagtgaccgccttgctcctgccgctggccttgctg
ctccacgccgccaggcattctagagcggaagtgcagctgcaggct
tccgggggaggatttgtgcagccgggggggtcattgcgactgagc
tgcgccgcatccggagcaacatcaaacatcagtaacatgggctgg
tttcgtcaggcccctggcaaggagagagagttcgtttccgccatc
tcccgtgcagaatcgcgtcctctgtattacgctgacagcgtaaag
ggaagatttacaattagccgggataactccaaaaacacggtctat
ctccagatgaacagcctcagggccgaggacacagctacgtattac
tgtgcatatatgcctctggttcgtcacaaggcatactggggacag
gggacgcaggtaactgtgagtagccctgcaggagagcagaagctg
atctcagaggaggacctgggccggccaCAGACGACTCCAGGAGAG
AGATCATCACTCCCTGCCTTTTACCCTGGCACTTCAGGCTCTTGT
TCCGGATGTGGGTCCCTCTCTCTGCCGCTCCTGGCAGGCCTCGTG
GCTGCTGATGCGGTGGCATCGCTGCTCATCGTGGGGGCGGTGTTC
CTGTGCGCACGCCCACGCCGCAGCCCCGCCCAAGAAGATGGCAAA
GTCTACATCAACATGCCAGGCAGGGGCCttaagAGAGTGAAGTTC
AGCAGGAGCGCAGACGCCCCCGCGTACCAGCAGGGCCAGAACCAG
CTCTATAACGAGCTCAATCTAGGACGAAGAGAGGAGTACGATGTT
TTGGACAAGAGACGTGGCCGGGACCCTGAGATGGGGGGAAAGCCG
CAGAGAAGGAAGAACCCTCAGGAAGGCCTGTACAATGAACTGCAG
AAAGATAAGATGGCGGAGGCCTACAGTGAGATTGGGATGAAAGGC
GAGCGCCGGAGGGGCAAGGGGCACGATGGCCTTTACCAGGGTCTC
AGTACAGCCACCAAGGACACCTACGACGCCCTTCACATGCAGGCC
CTGCCCCCTCGCaccggtggcCACGTTGTTGAAGGACTGGCTGGG
GAACTTGAACAACTTCGTGCACGACTGGAGCATCACCcacaaggt
caacgtgaaccaTGA
Features:
SP CD8: [1:61]
vHH: [70:429]
c-Myc tag: [439:468]
DAP10 extracellular: [478:567]
DAP10 TM: [568:630]
DAP10 cytosolic: [631:702]
CD3zeta domain site: [709:1047]
SBPs: [1051:1137]
HER2 sdAb1 DAP12 CAR
> [vHER2nº1-DAP12.xdna-837 bp] Fragment
extracted from pTRIP-SFFV-BFP-2a-vhhHER2-
Myc-DAP12-SBP.xdna [between 4504 and 5340]
atggccttaccagtgaccgccttgctcctgccgctggccttgctg
ctccacgccgccaggcattctagagcggaagtgcagctgcaggct
tccgggggaggatttgtgcagccgggggggtcattgcgactgagc
tgcgccgcatccggagcaacatcaaacatcagtaacatgggctgg
tttcgtcaggcccctggcaaggagagagagttcgtttccgccatc
tcccgtgcagaategcgtcctctgtattacgctgacagcgtaaag
ggaagatttacaattagccgggataactccaaaaacacggtctat
ctccagatgaacagcctcagggccgaggacacagctacgtattac
tgtgcatatatgcctctggttcgtcacaaggcatactggggacag
gggacgcaggtaactgtgagtagccctgcaggagagcagaagctg
atctcagaggaggacctgggccggccaCTCCGTCCTGTCCAGGCC
CAGGCCCAGAGCGATTGCAGTTGCTCTACGGTGAGCCCGGGCGTG
CTGGCAGGGATCGTGATGGGAGACCTGGTGCTGACAGTGCTCATT
GCCCTGGCCGTGTACTTCCTGGGCCGGCTGGTCCCTCGGGGGCGA
GGGGCTGCGGAGGCAGCGACCCGGAAACAGCGTATCACTGAGACC
GAGTCGCCTTATCAGGAGCTCCAGGGTCAGAGGTCGGATGTCTAC
AGCGACCTCAACACACAGAGGCCGTATTACAAACACGTTGTTGAA
GGACTGGCTGGGGAACTTGAACAACTTCGTGCACGACTGGAGCAT
CACccacaaggtcaacgtgaaccaTGA
Features:
SP CD8: [1:61]
vHH: [70:429]
Myc-Tag: [439:468]
DAP12 core: [478:534]
TransmembraneDAP12: [535:597]
DAP12 core: [598:753]
SBPdel−: [754:834]
Extracellular: [478:534]
TM: [535:597]
ITAM: [652:738]
HER2 sdAb nº2 41-z CAR
> [C8-n2vHHHER2 myc.xdna-1236 bp] Fragment
extracted from Sequence Window #8
[between4504and 5739]
atggccttaccagtgaccgccttgctcctgccgctggccttgctg
ctccacgccgccaggcattctagaGCGGAAGTGCAGCTGCAGGCT
TCCGGGGGAGGATTTGTGCAGCCGGGGGGGTCATTGCGACTGAGC
TGCGCCGCATCCGGAGATTCCTACAACGAGAGTTCTATGGGCTGG
TTTCGTCAGGCCCCTGGCAAGGAGAGAGAGTTCGTTTCCGCCATC
TCGGCACGTGGTAACCATCCTCTGTATTACGCTGACAGCGTAAAG
GGAAGATTTACAATTAGCCGGGATAACTCCAAAAACACGGTCTAT
CTCCAGATGAACAGCCTCAGGGCCGAGGACACAGCTACGTATTAC
TGTGCATCGATGCCTATGCCTAAGTGGAAGAAGTACTGGGGACAG
GGGACGCAGGTAACTGTGAGTAGCcctgcaggagagcagaagctg
atctcagaggaggacctgcatatgaccacgacgccagegccgcga
ccaccaacaceggegcccaccatcgcgtcgcagcccctgtccctg
cgcccagaggcgtgccggccagcggggggggcgcagtgcacacga
gggggctggacttcgcctgtgatatctacatctgggcgcccttgg
ccgggacttgtggggtccttctcctgtcactggttatcacccttt
actgcaaacggggcagaaagaaactcctgtatatattcaaacaac
catttatgagaccagtacaaactactcaagaggaagatggctgta
gctgccgatttccagaagaagaagaaggaggatgtgaactgagag
tgaagttcagcaggagcgcagacgcccccgcgtacaagcagggcc
agaaccagctctataacgagctcaatctaggacgaagagaggagt
acgatgttttggacaagagacgtggccgggaccctgagatggggg
gaaagccgagaaggaagaaccctcaggaaggcctgtacaatgaac
tgcagaaagataagatggcggaggcctacagtgagattgggatga
aaggcgagcgccggaggggcaaggggcacgatggcctttaccagg
gtctcagtacagccaccaaggacacctacgacgcccttcacatgc
aggccctgccccctcgcaccggtggccacgttgttgaaggactgg
ctggggaacttgaacaacttcgtgcacgactggagcatcacccac
aaggtcaacgtgaaccaTGA
Features:
SP CD8: [1:63]
VHH: [70:429]
c-Myc tag: [439:468]
hinge CD8: [475:609]
TmCD8: [610:681]
4-1BB: [682:807]
CD3zeta: [808:1143]
SBPs: [1147:1233]
scFv CD19 41-z CAR
> [scFv cD19 CAR.xdna-1596 bp] Fragment
extracted from pTRIP-SFFV-BFP-2a-
aCD19june-SBP [between 4504 and 6099]
ATGGCCTTACCAGTGACCGccttgctcctgccgctggccttgctg
ctccacgccgccaggccggatatccagatgacacagactacatcc
tccctgtctgcctctctgggagacagagtcaccatcagttgcagg
gcaagtcaggacattagtaaatatttaaattggtatcagcagaaa
ccagatggaactgttaaactcctgatctaccatacatcaagatta
cactcaggagtcccatcaaggttcagtggcagtgggtctggaaca
gattattctctcaccattagcaacctggagcaagaagatattgcc
acttacttttgccaacagggtaatacgcttccgtacacgttcgga
ggggggaccaagctggagatcacaggtggcggtggctcgggcggt
ggtgggtcgggtggcggcggatctgaggtgaaactgcaggagtca
ggacctggcctggtggcgccctcacagagcctgtccgtcacatgc
actgtctcaggggtctcattacccgactatggtgtaagctggatt
cgccagcctccacgaaagggtctggagtggctgggagtaatatgg
ggtagtgaaaccacatactataattcagctctcaaatccagactg
accatcatcaaggacaactccaagagccaagttttcttaaaaatg
aacagtctgcaaactgatgacacagccatttactactgtgccaaa
cattattactacggtggtagctatgctatggactactggggccaa
ggaacctcagtcaccgtctcctcacctgcaggagagcagaagctg
atctcagaggaggacctgcatatgaccacgacgccagcgccgcga
ccaccaacaccggcgcccaccatcgcgtcgcagcccctgtccctg
cgcccagaggcgtgccggccagcggcggggggcgcagtgcacacg
agggggctggacttcgcctgtgatatctacatctgggcgcccttg
gccgggacttgtggggtccttctcctgtcactggttatcaccctt
tactgcaaacggggcagaaagaaactectgtatatattcaaacaa
ccatttatgagaccagtacaaactactcaagaggaagatggctgt
agctgccgatttccagaagaagaagaaggaggatgtgaactgaga
gtgaagttcagcaggagcgcagacgcccccgcgtacaagcagggc
cagaaccagctctataacgagctcaatctaggacgaagagaggag
tacgatgttttggacaagagacgtggccgggaccctgagatgggg
ggaaagccgagaaggaagaaccctcaggaaggcctgtacaatgaa
ctgcagaaagataagatggcggaggcctacagtgagattgggatg
aaaggcgagcgccggaggggcaaggggcacgatggcctttaccag
ggtctcagtacagccaccaaggacacctacgacgcccttcacatg
caggccctgccccctcgcaccggtggccacgttgttgaaggactg
gctggggaacttgaacaacttcgtgcacgactggagcatcaccca
caaggtcaacgtgaaccaTGA
Features:
CD8ss-signal peptide: [1:63]
scFv-CD19: [64:789]
Myc-Tag: [799:828]
Hinge CD8: [832:966]
Tm CD8: [967:1040]
AD 4-1BB: [1039:1164]
AD CD3z: [1165:1500]
ITAM1: [1195:1281]
ITAM2: [1315:1395]
ITAM3: [1402:1488]
SBPdel−: [1513:1593]

1. A humanized synthetic single domain antibody (hssdAb) directed against HER2 (anti-HER2 sdAb), wherein said anti-HER2 sdAb has the following formula FR1-CDR1-FR2-CDR2-FR3-CDR3-FR4, and wherein the CDRs are selected from:
a. a CDR1 of SEQ ID NO:1; a CDR2 of SEQ ID NO:2 and a CDR3 of SEQ ID NO:3,
b. a CDR1 of SEQ ID NO:4; a CDR2 of SEQ ID NO:5 and a CDR3 of SEQ ID NO:6,
c. a CDR1 of SEQ ID NO:7; a CDR2 of SEQ ID NO:8 and a CDR3 of SEQ ID NO:9,
d. a CDR1 of SEQ ID NO:10; a CDR2 of SEQ ID NO:11 and a CDR3 of SEQ ID NO:12,
e. a CDR1 of SEQ ID NO:13; a CDR2 of SEQ ID NO:14 and a CDR3 of SEQ ID NO:15,
f. a CDR1 of SEQ ID NO:16; a CDR2 of SEQ ID NO:17 and a CDR3 of SEQ ID NO:18, or
g. CDR1, CDR2 and CDR3 as defined in any one of a-f further having one or more conservative amino acid modifications in one or more of these CDRs.

2. The anti-HER2 sdAb according to claim 1 comprising:
a sequence selected from the group consisting of SEQ ID NO: 23, SEQ ID NO:24, SEQ ID NO:25, SEQ ID NO:26, SEQ ID NO:27, and SEQ ID NO:28: or
a sequence having at least 90% identity with a sequence selected from the group consisting of SEQ ID NO: 23, SEQ ID NO:24, SEQ ID NO:25, SEQ ID NO:26, SEQ ID NO:27, and SEQ ID NO:28.

3. The anti-HER2 sdAb according to claim 1 which is linked directly or indirectly, covalently or non-covalently to a compound of interest selected from a nucleic acid, a polypeptide or a protein, a virus, a toxin and a chemical entity.

4. The anti-HER2 sdAb according to claim 1 which is fused to an immunoglobulin domain.

5. A multivalent binding compound comprising at least a first sdAb consisting in the anti-HER2 sdAb according to claim 1, and comprising at least a second antigen binding compound directed against an antigen selected from a polypeptide, a protein or a small molecule.

6. A chimeric antigen receptor (CAR) comprising:
(a) an antigen binding domain comprising at least a first sdAb consisting in the anti-HER2 sdAb according to claim 1, wherein said sdAb comprises CDR sequences as defined in claim 1a, b, d-f or has a sequence selected from the group consisting of SEQ ID NO: 23, SEQ ID NO:24, SEQ ID NO:26, SEQ ID NO:27, and SEQ ID NO:28;
a sequence having at least 90% identity with a sequence selected from the group consisting of SEQ ID NO: 23, SEQ ID NO:24, SEQ ID NO:26, SEQ ID NO:27, and SEQ ID NO:28;
a CDR1 of SEQ ID NO:1; a CDR2 of SEQ ID NO:2 and a CDR3 of SEQ ID NO:3 and further having one or more conservative amino acid modifications in one or more of these CDRs;
a CDR1 of SEQ ID NO:4; a CDR2 of SEQ ID NO:5 and a CDR3 of SEQ ID NO:6 and further having one or more conservative amino acid modifications in one or more of these CDRs.
a CDR1 of SEQ ID NO:10; a CDR2 of SEQ ID NO:11 and a CDR3 of SEQ ID NO:12 and further having one or more conservative amino acid modifications in one or more of these CDRs,
(b) a transmembrane domain; and
(c) an intracellular domain.

7. A multivalent binding compound or a CAR comprising at least a first sdAb consisting in the anti-HER2 sdAb according to claim 1, wherein a second antigen is selected from the group consisting of typically antigens other than HER2 can be selected from PSMA, PSCA, BCMA, CS1, GPC3, CSPG4, EGFR, HER3, CA125, CD 123, 5T4, IL-13R, CD2, CD3, CD16 (FcγRIII), CD19, CD20, CD22, CD33, CD23, L1 CAM, MUC16, ROR1, SLAMF7, cKit, CD38, CD53, CD71, CD74, CD92, CD100, CD123, CD138, CD146 (MUC18), CD148, CD150, CD200, CD261, CD262, CD362, ROR1, mesothelin, CD33/IL3Ra, c-Met, Glycolipid F77, EGFRvlll, MART-1, gp100, GD-2, 0-GD2, NKp46 receptor, presented antigens like NY-ESO-1 or MAGE A3, human telomerase reverse transcriptase (hTERT), survivin, cytochrome P450 1 B1 (CY1 B), Wilm's tumor gene 1 (WT1), livin, alphafetoprotein (AFP), carcinoembryonic antigen (CEA), mucin 16, MUC1, p53, cyclin, and an immune checkpoint target or combinations thereof.

8. A CAR comprising at least a first sdAb consisting in the anti-HER2 sdAb according to claim 1, wherein the transmembrane domain is selected from CD8, CD28, DAP10 and DAP12 and the intracellular domain comprises one or more domains derived from the group selected from the CD3 zeta chain intracellular domain, the CD28 intracellular domain, the 4-1BB intracellular domain; the DAP10 intracellular domain or the DAP12 intracellular domain.

9. An isolated nucleic acid comprising a nucleic acid sequence encoding the humanized anti-HER2 sdAb according to claim 1.

10. A vector comprising a nucleic acid comprising a nucleic acid sequence encoding the humanized anti-HER2 sdAb according to claim 1.

11. A host cell comprising a nucleic acid comprising a nucleic acid sequence encoding the humanized anti-HER2 sdAb according to claim 1.

12. An isolated cell or population of cells expressing the humanized anti-HER2 SdAb according to claim 1.

13. (canceled)

14. (canceled)

15. An in vitro or ex vivo method for diagnosing or monitoring an HER2 mediated cancer in a subject comprising the steps of:
a) contacting in vitro an appropriate sample from said subject with a diagnostic agent comprising the anti-HER2 sdAb according to claim 1, and
b) determining the expression of HER2 in said sample.

16. The anti-HER2 sdAb according to claim 1 wherein said anti HER2 sdAb is linked directly or indirectly, covalently or non-covalently to a diagnostic compound selected from an enzyme, a fluorophore, a NMR or MRI contrast agent, a radioisotope and a nanoparticle.

17. The anti-HER2 sdAb according to claim 1 wherein said said anti HER2 sdAb is linked directly or indirectly, covalently or non-covalently to a therapeutic compound selected from cytotoxic agents, chemotherapeutic agents, radioisotopes, targeted anti-cancer agents, immunotherapeutic agents, and lytic peptides.

18. The anti-HER2 sdAb according to claim 1 which is fused to an Fc domain.

19. A CAR comprising at least a first sdAb consisting in the anti-HER2 sdAb according to claim 1 wherein
the CAR comprises the full DAP12 protein, or a fragment thereof having at least 90% identity with the DAP12 protein,
the CAR comprises the full DAP10 protein or a fragment thereof having at least 90% identity with the DAP10 protein and the CD3zeta intracellular domain, or
the CAR comprises the 4-1BB and CD3 zeta intracellular domains.

20. An isolated cell or population of cells expressing the humanized anti-HER2 SdAb according to claim 1 wherein the isolated cell or population of cells is or comprises an immune cell.

21. A method for the treatment of cancer in a subject in need thereof comprising administering a therapeutically efficient amount of the humanized anti-HER2 SdAb according to claim 1.

22. A method for the treatment of cancer in a subject in need thereof comprising administering a therapeutically efficient amount of the humanized anti-HER2 SdAb according to claim 1 wherein said humanized anti-HER2 sdAb is used in combination with at least one further therapeutic agent, wherein said at least one further therapeutic agent is an anticancer agent.
